(19) United States
(12) Patent Application Publication
(10) Pub. No.: US 2022/0289728 A1
(43) Pub. Date: Sep. 15, 2022

(54) MODULATORS OF TREX1

(71) Applicant: Constellation Pharmaceuticals, Inc., Cambridge, MA (US)

(72) Inventors: Avinash Khanna, Cambridge, MA (US); Jonathan E. Wilson, Arlington, MA (US)

(21) Appl. No.: 17/628,946

(22) PCT Filed: Jul. 22, 2020
(86) PCT No.: PCT/US2020/043012

§ 371 Date: Jan. 21, 2022

Related U.S. Application Data
(60) Provisional application No. 62/877,482, filed on Jul. 23, 2019.

(51) Int. Cl. C07D 413/12; C07D 413/14; C07D 417/14

(52) U.S. Cl. CPC C07D 413/12; C07D 413/14; C07D 417/14

(57) ABSTRACT

Provided are compounds of Formula (I): and pharmaceutically acceptable salts and compositions thereof, which are useful for treating a variety of conditions associated with TREX1.

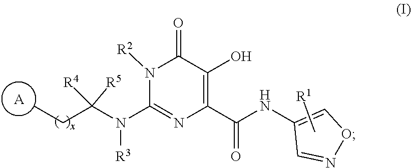

### RELATED APPLICATIONS

This application claims the benefit of priority to U.S. Provisional Application No. 62/877,482 filed Jul. 23, 2019, the entire contents of which are incorporated herein by reference.

### BACKGROUND

A potential immune therapy is needed for cancers related to the innate immune system recognition of non-self, and to detect and protect against potential danger. Cancer cells differ antigenically from their normal counterparts and emit danger signals to alert the immune system similar to viral infection. These signals, which include damage-associated molecular patterns (DAMPs) and pathogen-associated molecular patterns (PAMPs), further activate the innate immune system resulting in the protection of the host from a variety of threats (Front. Cell Infect. Microbiol. 2012, 2, 168).

Ectopically expressed single stranded DNA (ssDNA) and double stranded DNA (dsDNA) are known PAMPs and/or DAMPs, which are being recognized by the cyclic GMP-AMP synthase (cGAS), a nucleic acid sensor (Nature 2011, 478, 515-518). Upon sensing of cytosolic DNA, cGAS catalyzes the generation of the cyclic dinucleotide 2′,3′-cGAMP, a potent second messenger and activator of the ER transmembrane adapter protein stimulator of interferon genes (STING) (Cell Rep. 2013, 3, 1355-1361). STING activation triggers phosphorylation of IRF3 via TBK1 which in turn leads to type I interferon production and activation of interferon stimulated genes (ISGs); a pre-requisite to the activation of innate immunity and initiation of adaptive immunity. Production of type I interferons thus constitutes a key bridge between the innate and adaptive immunity (Science 2013, 341, 903-906).

Excess type I IFN can be harmful to the host and induce autoimmunity, therefore, negative feedback mechanisms exist that keep type I IFN-mediated immune activation in check. Three prime repair exonuclease I (TREX1) is a 3′-5′ DNA exonuclease responsible for the removal of ectopically expressed ssDNA and dsDNA and is therefore a key repressor of the cGAS/STING pathway (PNAS 2015, 112, 5117-5122).

Type I interferons and downstream pro-inflammatory cytokine responses are critical to the development of immune responses and their effectiveness. Type I interferons enhance both the ability of dendritic cells and macrophages to take up, process, present, and cross-present antigens to T cells, and their potency to stimulate T cells by eliciting the up-regulation of the co-stimulatory molecules such as CD40, CD80 and CD86 (J. Exp. Med. 2011, 208, 2005-2016). Type I interferons also bind their own receptors and activate interferon responsive genes that contribute to activation of cells involved in adaptive immunity (EMBO Rep. 2015, 16, 202-212).

From a therapeutic perspective, type I interferons and compounds that can induce type I interferon production have potential for use in the treatment of human cancers (Nat. Rev Immunol. 2015, 15, 405-414). Interferons can inhibit human tumor cell proliferation directly. In addition, type I interferons can enhance anti-tumor immunity by triggering the activation of cells from both the innate and adaptive immune system. Importantly, the anti-tumor activity of PD-1 blockade requires pre-existing intratumoral T cells. By turning cold tumors into hot and thereby eliciting a spontaneous anti-tumor immunity, type I IFN-inducing therapies have the potential to expand the pool of patients responding to anti-PD-1 therapy as well as enhance the effectiveness of anti-PD1 therapy.

Therapies that are currently in development that induce a potent type I interferon response require focal or intratumoral administration to achieve an acceptable therapeutic index. Thus, there remains a need for new agents with systemic delivery and lower toxicity to expand the benefit of type I IFN-inducing therapies to patients without peripherally treatment accessible lesions. Human and mouse genetic studies suggest that TREX1 inhibition might be amenable to a systemic delivery route and therefore TREX1 inhibitory compounds could play an important role in the anti-tumor therapy landscape. TREX1 is a key determinant for the limited immunogenicity of cancer cells responding to radiation treatment [Trends in Cell Biol., 2017, 27 (8), 543-4; Nature Commun., 2017, 8, 15618]. TREX1 is induced by genotoxic stress and involved in protection of glioma and melanoma cells to anticancer drugs [Biochim. Biophys. Acta, 2013, 1833, 1832-43]. STACT-TREX1 therapy shows robust anti-tumor efficacy in multiple murine cancer models [Glickman et al, Poster P235, 33^{rd} Annual Meeting of Society for Immunotherapy of Cancer, Washington D.C., Nov. 7-11, 2018].

### SUMMARY

Provided herein are compounds having the Formula I:

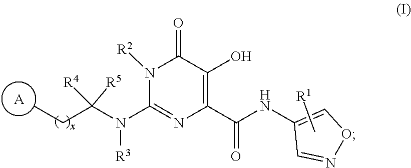

and pharmaceutically acceptable salts and compositions thereof, wherein R¹, R², R³, R⁴, R⁵, x, and ring A are as described herein. The disclosed compounds and compositions modulate TREX1, and are useful in a variety of therapeutic applications such as, for example, in treating cancer.

### BRIEF DESCRIPTION OF THE FIGURES

FIG. 1A illustrates the results from a knock down experiment of TREX1 in B16F10 tumor cells using CRISPR. FIG. 1B illustrates TREX1 attenuated the activation of the cGAS/STING pathway in B16F10 tumor cells.

FIG. 2. illustrates that tumors in which TREX had been silenced had smaller volumes compared with parental B16F10 tumors.

FIG. 3. shows that TREX1 knockout B16F10 tumors exhibited a significant increase in overall immune cells. This reflected an increase in the number of tumor infiltrating CD4 and CD8 T cells as well as in plasmacytoid dendritic cells (pDCs).

FIG. 4 shows the results from a luciferase assay using a compound described herein in a HCT116 colorectal carcinoma cell line.

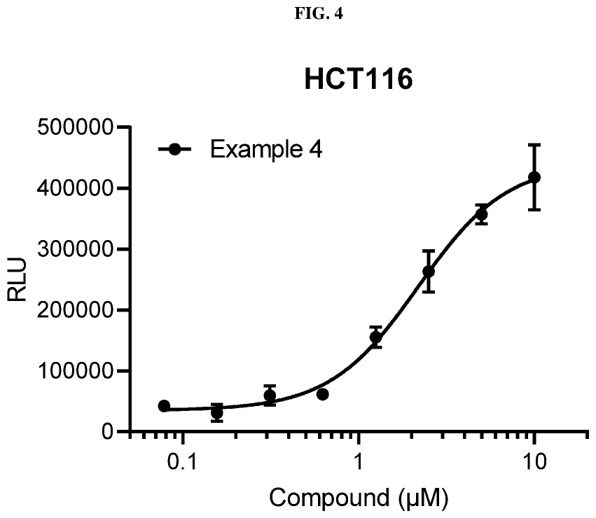

### DETAILED DESCRIPTION

### 1. General Description of Compounds

In a first embodiment, provided herein is a compound of Formula I:

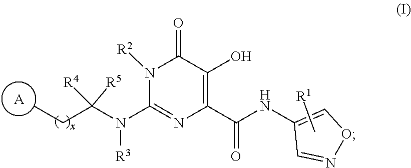

or a pharmaceutically acceptable salt thereof, wherein:

R¹ is hydrogen, (C₁-C₄)alkyl, halo(C₁-C₄)alkyl, 3- to 4-membered cycloalkyl, —OR^{f}, —SR^{f}, or —NR^{e}R^{f};

R² is hydrogen, (C₁-C₄)alkyl, halo(C₁-C₄)alkyl, or 3- to 4-membered cycloalkyl;

R³ is hydrogen or (C₁-C₄)alkyl optionally substituted with phenyl, wherein said phenyl is optionally substituted with 1 to 3 groups selected from halo, (C₁-C₄)alkyl, and halo(C₁-C₄)alkyl;

R⁴ is hydrogen or (C₁-C₄)alkyl;

R⁵ is hydrogen, aryl, heteroaryl, heterocyclyl, cycloalkyl, phenyl, or (C₁-C₄)alkyl optionally substituted with phenyl or —NHC(O)OR^{a}, wherein each of said phenyl is optionally and independently substituted with 1 to 3 groups selected from halo, (C₁-C₄)alkyl, and halo(C₁-C₄)alkyl;

x is 0, 1, or 2;

Ring A is aryl, heteroaryl, heterocyclyl, or cycloalkyl, each of which are optionally and independently substituted with 1 or 2 groups selected from R⁶;

R⁶ is (C₁-C₄)alkyl, halo(C₁-C₄)alkyl, halo(C₁-C₄)alkoxy, halo, phenyl, —CN, —NHC(O)OR^{a}, —NHC(S)OR^{a}, —C(O)R^{b}, —NHC(O)NHR^{g}, —NHC(S)NHR^{g}, —NHS(O)₂NHR^{g}, —C(S)R^{b}, —S(O)₂R^{c}, —S(O)R^{c}, —C(O)OR^{d}, —C(S)OR^{d}, —C(O)NR^{e}R^{f}, —C(S)NHR^{e}, —NHC(O)R^{d}, —NHC(S)R^{d}, —OR^{e}, —SR^{e}, —O(C₁-C₄)alkylOR^{e}, —NR^{e}R^{f}, 4- to 6-membered heteroaryl, or 4- to 7-membered heterocyclyl, wherein
said phenyl for R⁶ is optionally substituted with 1 or 2 groups selected from R^{g};
said (C₁-C₄)alkyl for R⁶ is optionally substituted with 1 or 2 groups selected from OR^{h}, —NR^{j}R^{k}, phenyl, and 5- to 6-membered heteroaryl; and
said 4- to 7-membered heterocyclyl and 4- to 6-membered heteroaryl for R⁶ are each optionally and independently substituted with 1 or 2 groups selected from R^{m}; and wherein said phenyl and 5- to 6-membered heteroaryl of the optional substituents listed for (C₁-C₄)alkyl in R⁶ are each optionally and independently substituted with 1 or 2 groups selected from R^{g};

R^{g}, R^{h}, R^{j}, R^{k}, and R^{m} are each independently hydrogen, halo, (C₁-C₄)alkyl, halo(C₁-C₄)alkyl, (C₁-C₄)alkoxy, halo(C₁-C₄)alkoxy, phenyl, —(C₁-C₄)alkylphenyl, 3- to 4-membered cycloalkyl, 4- to 6-membered heteroaryl, or 4- to 7-membered heterocyclyl, and wherein said 4- to 7-membered heterocyclyl for R^{g}, R^{h}, R^{j} and R^{k} is further optionally substituted with ═O.

R^{a}, R^{b}, R^{c}, R^{d}, R^{e} and R^{f} are each independently hydrogen, halo, (C₁-C₄)alkyl, halo(C₁-C₄)alkyl, (C₁-C₄)alkoxy, halo(C₁-C₄)alkoxy, phenyl, 3- to 4-membered cycloalkyl, 4-to 6-membered heteroaryl, or 4- to 7-membered heterocyclyl, wherein
said (C₁-C₄)alkyl for R^{a}, R^{b}, R^{c}, R^{d}, R^{e} and R^{f} is optionally substituted with 1 or 2 groups selected from phenyl, —OR^{h}, —NR^{j}R^{k},
said phenyl, 4- to 6-membered heteroaryl, and 4- to 7-membered heterocyclyl for R^{a}, R^{b}, R^{c}, R^{d}, R^{e}, and R^{f} are each optionally and independently substituted with 1 or 2 groups selected from R^{g}, and
said 4- to 7-membered heterocyclyl for R^{a}, R^{b}, R^{c}, R^{d}, R^{e}, and R^{f} is further optionally substituted with ═O.

### 2. Definitions

When used in connection to describe a chemical group that may have multiple points of attachment, a hyphen (-) designates the point of attachment of that group to the variable to which it is defined. For example, —NHC(O)OR^{a} and —NHC(S)OR^{a} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical. Unless otherwise specified, an alkyl group typically has 1-4 carbon atoms, i.e., (C₁-C₄)alkyl.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term “aryl” refers to an aromatic carbocyclic ring system having, unless otherwise specified, a total of 6 to 10 ring members. In certain embodiments, “aryl” refers to an aromatic ring system which includes, but is not limited to, phenyl and naphthyl. It will be understood that when specified, optional substituents on an aryl group may be present on any substitutable position and, include, e.g., the position at which the aryl is attached.

The term “heteroaryl” used alone or as part of a larger moiety refers to a 5- to 12-membered (e.g., a 5- to 6-membered) aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered saturated or partially unsaturated heterocyclic ring containing 1 to 4 heteroatoms independently selected from N, O, and S. A heterocyclyl ring can be attached to its pendant group at any heteroatom or carbon atom that results in a stable structure. A heterocyclyl group may be mono- or bicyclic. Examples of monocyclic saturated or partially unsaturated heterocyclic radicals include, without limitation, tetrahydrofuranyl, tetrahydrothienyl, tetrahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, and tetrahydropyrimidinyl. Bi-cyclic heterocyclyl groups include, e.g., unsaturated heterocyclic radicals fused to another unsaturated heterocyclic radical, cycloalkyl, or aromatic or heteroaryl ring, such as for example, benzodioxolyl, dihydrobenzooxazinyl, dihydrobenzodioxinyl, 6,7-dihydro-5H-pyrrolo[2,1-c][1,2,4]triazolyl, 5,6,7,8-tetrahydroimidazo[1,2-a]pyridinyl, 1,2-dihydroquinolinyl, dihydrobenzofuranyl, tetrahydronaphthyridine, indolinone, dihydropyrrolotriazole, quinolinone, chromanyl, and dioxaspirodecane. It will be understood that when specified, optional substituents on a heterocyclyl group may be present on any substitutable position and, include, e.g., the position at which the heterocyclyl is attached.

The term “spiro” refers to two rings that shares one ring atom (e.g., carbon).

The term “fused” refers to two rings that share two adjacent ring atoms with one another.

The term “bridged” refers to two rings that share three ring atoms with one another.

The term “cycloalkyl” refers to a cyclic hydrocarbon having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl or cycloaliphatic group is attached.

The disclosed compounds exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that contain two or more asymmetrically substituted carbon atoms. “R” and “S” represent the configuration of substituents around one or more chiral carbon atoms.

“Racemate” or “racemic mixture” means a compound of equimolar quantities of two enantiomers, wherein such mixtures exhibit no optical activity, i.e., they do not rotate the plane of polarized light.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomer is at least 60%, 70%, 80%, 90%, 99% or 99.9% by weight pure relative to all of the other stereoisomers. The stereoisomeric purity in this case is determined by dividing the total weight in the mixture of the stereoisomers encompassed by the name or structure by the total weight in the mixture of all of the stereoisomers.

When a disclosed compound is named or depicted by structure without indicating the stereochemistry, and the compound has one chiral center, it is to be understood that the name or structure encompasses one enantiomer of compound free from the corresponding optical isomer, a racemic mixture of the compound, or mixtures enriched in one enantiomer relative to its corresponding optical isomer.

When a disclosed compound is named or depicted by structure without indicating the stereochemistry and e.g., the compound has more than one chiral center (e.g., at least two chiral centers), it is to be understood that the name or structure encompasses one stereoisomer free of other stereoisomers, mixtures of stereoisomers, or mixtures of stereoisomers in which one or more stereoisomers is enriched relative to the other stereoisomer(s). For example, the name or structure may encompass one stereoisomer free of other diastereomers, mixtures of stereoisomers, or mixtures of stereoisomers in which one or more diastereomers is enriched relative to the other diastereomer(s).

The term “TREX1” refers to three prime repair exonuclease 1 or DNA repair exonuclease 1, which is an enzyme that in humans is encoded by the TREX1 gene. Mazur D J, Perrino F W (August 1999). “Identification and expression of the TREX1 and TREX2 cDNA sequences encoding mammalian 3′-->5′ exonucleases”. J Biol Chem. 274 (28): 19655-60. doi:10.1074/jbc.274.28.19655. PMID 10391904; Hoss M, Robins P, Naven T J, Pappin D J, Sgouros J, Lindahl T (August 1999). “A human DNA editing enzyme homologous to the Escherichia coli DnaQ/MutD protein”. EMBO J. 18 (13): 3868-75. doi:10.1093/emboj/18.13.3868. PMC 1171463. PMID 10393201. This gene encodes the major 3′->5′ DNA exonuclease in human cells. The protein is a non-processive exonuclease that may serve a proofreading function for a human DNA polymerase. It is also a component of the SET complex, and acts to rapidly degrade 3′ ends of nicked DNA during granzyme A-mediated cell death. Cells lacking functional TREX1 show chronic DNA damage checkpoint activation and extra-nuclear accumulation of an endogenous single-strand DNA substrate. It appears that TREX1 protein normally acts on a single-stranded DNA polynucleotide species generated from processing aberrant replication intermediates. This action of TREX1 attenuates DNA damage checkpoint signaling and prevents pathological immune activation. TREX1 metabolizes reverse-transcribed single-stranded DNA of endogenous retroelements as a function of cell-intrinsic antiviral surveillance, resulting in a potent type I IFN response. TREX1 helps HIV-1 to evade cytosolic sensing by degrading viral cDNA in the cytoplasm.

The term “TREX2” refers to Three prime repair exonuclease 2 is an enzyme that in humans is encoded by the TREX2 gene. This gene encodes a nuclear protein with 3′ to 5′ exonuclease activity. The encoded protein participates in double-stranded DNA break repair, and may interact with DNA polymerase delta. Enzymes with this activity are involved in DNA replication, repair, and recombination. TREX2 is a 3′-exonuclease which is predominantly expressed in keratinocytes and contributes to the epidermal response to UVB-induced DNA damage. TREX2 biochemical and structural properties are similar to TREX1, although they are not identical. The two proteins share a dimeric structure and can process ssDNA and dsDNA substrates in vitro with almost identical k_{cat} values. However, several features related to enzyme kinetics, structural domains, and subcellular distribution distinguish TREX2 from TREX1. TREX2 present a 10-fold lower affinity for DNA substrates in vitro compared with TREX1. In contrast with TREX1, TREX2 lacks a COOH-terminal domain that can mediate protein-protein interactions. TREX2 is localized in both the cytoplasm and nucleus, whereas TREX1 is found in the endoplasmic reticulum, and is mobilized to the nucleus during granzyme A—mediated cell death or after DNA damage.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, provided herein is a compound of Formula II:

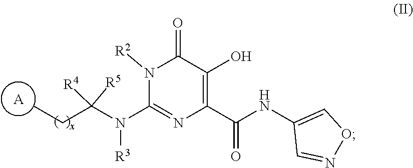

or a pharmaceutically acceptable salt thereof, wherein the variables are as described above for Formula I.

In a third embodiment, R² is (C₁-C₄)alkyl in the compounds of Formula I or II.

In a fourth embodiment, provided herein is a compound of Formula III:

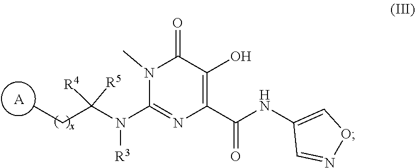

or a pharmaceutically acceptable salt thereof, wherein the variables are as described above in the first, second, or third embodiment.

In a fifth embodiment, R³ in the compounds of Formula I, II, or III is (C₁-C₄)alkyl optionally substituted with phenyl, wherein the variables are as described above in the first, second, third, or fourth embodiment. Alternatively, R³ in the compounds of Formula I, II, or III is (C₁-C₄)alkyl, wherein the variables are as described above in the first, second, third, or fourth embodiment.

In a sixth embodiment, provided herein is a compound of Formula IV:

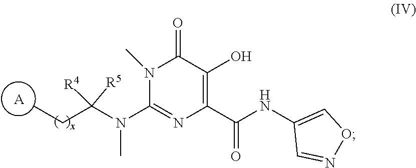

or a pharmaceutically acceptable salt thereof, wherein the variables are as described above in the first, second, third, fourth, or fifth embodiment.

In a seventh embodiment, provided herein is a compound of Formula V:

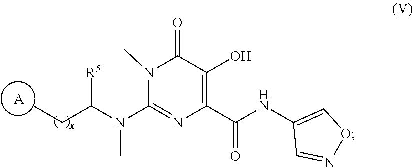

or a pharmaceutically acceptable salt thereof, wherein the variables are as described above in the first, second, third, fourth, fifth, or sixth embodiment.

In an eighth embodiment, x in the compounds of Formula I, II, III, IV, or V is 0 or 1, wherein the variables are as described above in the first, second, third, fourth, fifth, sixth, or seventh embodiment.

In a ninth embodiment, R⁵ in the compounds of Formula I, II, III, IV, or V is hydrogen, aryl, heteroaryl, heterocyclyl, cycloalkyl, phenyl, or (C₁-C₄)alkyl optionally substituted with phenyl or —NHC(O)OR^{a}, wherein the variables are as described above in the first, second, third, fourth, fifth, sixth, seventh, or eighth embodiment. Alternatively, as part of a ninth embodiment, R⁵ in the compounds of Formula I, II, III, IV, or V is hydrogen, phenyl, or (C₁-C₄)alkyl optionally substituted with phenyl or —NHC(O)OR^{a}, wherein the variables are as described above in the first, second, third, fourth, fifth, sixth, seventh, or eighth embodiment. Alternatively, as part of a ninth embodiment, R⁵ in the compounds of Formula I, II, III, IV, or V is cycloalkyl or phenyl, wherein said phenyl is optionally substituted with 1 to 3 groups selected from halo, (C₁-C₄)alkyl, and halo(C₁-C₄)alkyl, wherein the variables are as described above in the first, second, third, fourth, fifth, sixth, seventh, or eighth embodiment. Alternatively, as part of a ninth embodiment, R⁵ in the compounds of Formula I, II, III, IV, or V is cyclopropyl, wherein the variables are as described above in the first, second, third, fourth, fifth, sixth, seventh, or eighth embodiment. Alternatively, as part of a ninth embodiment, R⁵ in the compounds of Formula I, II, III, IV, or V is phenyl optionally substituted with 1 to 2 groups selected from halo and (C₁-C₄)alkyl, and halo(C₁-C₄)alkyl, wherein the variables are as described above in the first, second, third, fourth, fifth, sixth, seventh, or eighth embodiment. Alternatively, as part of a ninth embodiment, R⁵ in the compounds of Formula I, II, III, IV, or V is phenyl optionally substituted with 1 to 2 halo, wherein the variables are as described above in the first, second, third, fourth, fifth, sixth, seventh, or eighth embodiment.

In a tenth embodiment, R^{a} in the compounds of Formula I, II, III, IV, or V is (C₁-C₄)alkyl, wherein the variables are as described above in the first, second, third, fourth, fifth, sixth, seventh, eighth, or ninth embodiment.

In an eleventh embodiment, ring A in the compounds of Formula I, II, III, IV, or V is aryl, heteroaryl, or heterocyclyl, each of which are optionally and independently substituted with 1 or 2 groups selected from R⁶, wherein the variables are as described above in the first, second, third, fourth, fifth, sixth, seventh, eighth, ninth, or tenth embodiment. Alternatively, ring A in the compounds of Formula I, II, III, IV, or V is naphthalenyl, indazolyl, phenyl, pyridyl, pyrazolyl, azetidinyl, tetrahydropyranyl, piperidinyl, dihydrobenzooxazinyl, dihydrobenzodioxinyl, or chromanyl, each of which are optionally and independently substituted with 1 or 2 groups selected from R⁶, wherein the variables are as described above in the first, second, third, fourth, fifth, sixth, seventh, eighth, ninth, or tenth embodiment. In another alternative, ring A in the compounds of Formula I, II, III, IV, or V is phenyl optionally substituted with 1 or 2 groups selected from R⁶, wherein the variables are as described above in the first, second, third, fourth, fifth, sixth, seventh, eighth, ninth, or tenth embodiment. In another alternative, ring A in the compounds of Formula I, II, III, IV, or V is pyrimidinyl or thiazolyl each of which being optionally substituted with 1 or 2 groups selected from R⁶, wherein the variables are as described above in the first, second, third, fourth, fifth, sixth, seventh, eighth, ninth, or tenth embodiment. In another alternative, ring A in the compounds of Formula I, II, III, IV, or V is pyridyl optionally substituted with 1 or 2 groups selected from R⁶, wherein the variables are as described above in the first, second, third, fourth, fifth, sixth, seventh, eighth, ninth, or tenth embodiment.

In a twelfth embodiment, R⁶ in the compounds of Formula I, II, III, IV, or V is halo(C₁-C₄)alkyl, halo, —CN, —NHC(O)OR^{a}, —C(O)R^{b}, —NHC(O)NHR^{g}, —C(O)NR^{e}R^{f}, —NHC(O)R^{d}, —NR^{e}R^{f}, —OR^{e}, or 4- to 6-membered heteroaryl, wherein said 4- to 6-membered heteroaryl is optionally substituted with 1 or 2 groups selected from R^{m}, wherein the variables are as described above in the first, second, third, fourth, fifth, sixth, seventh, eighth, ninth, tenth, or eleventh embodiment. Alternatively, R⁶ in the compounds of Formula I, II, III, IV, or V is (C₁-C₄)alkyl, halo(C₁-C₄)alkyl, halo, —CN, —C(O)R^{b}, —C(O)NR^{e}R^{f}, —OR^{e}, or 4- to 6-membered heteroaryl, wherein said 4- to 6-membered heteroaryl is optionally substituted with 1 or 2 groups selected from R^{m}, wherein the variables are as described above in the first, second, third, fourth, fifth, sixth, seventh, eighth, ninth, tenth, or eleventh embodiment. Alternatively, R⁶ in the compounds of Formula I, II, III, IV, or V is phenyl or 4- to 6-membered heteroaryl, wherein said phenyl for is optionally substituted with 1 or 2 groups selected from R^{g} and said 4- to 6-membered heteroaryl is optionally substituted with 1 or 2 groups selected from R^{m}, wherein the variables are as described above in the first, second, third, fourth, fifth, sixth, seventh, eighth, ninth, tenth, or eleventh embodiment.

In a thirteenth embodiment, R^{b} in the compounds of Formula I, II, III, IV, or V is (C₁-C₄)alkyl, wherein the variables are as described above in the first, second, third, fourth, fifth, sixth, seventh, eighth, ninth, tenth, eleventh, or twelfth embodiment.

In a fourteenth embodiment, R^{e} in the compounds of Formula I, II, III, IV, or V is (C₁-C₄)alkyl, wherein the variables are as described above in the first, second, third, fourth, fifth, sixth, seventh, eighth, ninth, tenth, eleventh, twelfth, or thirteenth embodiment.

In a fifteenth embodiment, R^{r} in the compounds of Formula I, II, III, IV, or V is (C₁-C₄)alkyl, wherein the variables are as described above in the first, second, third, fourth, fifth, sixth, seventh, eighth, ninth, tenth, eleventh, twelfth, thirteenth, or fourteenth embodiment.

In a sixteenth embodiment, R^{m} in the compounds of Formula I, II, III, IV, or V is (C₁-C₄)alkyl, wherein the variables are as described above in the first, second, third, fourth, fifth, sixth, seventh, eighth, ninth, tenth, eleventh, twelfth, thirteenth, fourteenth, or fifteenth embodiment.

In a seventeenth embodiment, R^{g} in the compounds of Formula I, II, III, IV, or V is halo, wherein the variables are as described above in the first, second, third, fourth, fifth, sixth, seventh, eighth, ninth, tenth, eleventh, twelfth, thirteenth, fourteenth, fifteenth, or sixteenth embodiment.

In a eighteenth embodiment, R⁶ in the compounds of Formula I, II, III, IV, or V is Cl, F, CF₃, —C(O)N(Me)₂, —OCH₃, —C(O)CH₃, or pyrazolyl optionally substituted with 1 or 2 CH₃, wherein the variables are as described above in the first, second, third, fourth, fifth, sixth, seventh, eighth, ninth, tenth, eleventh, twelfth, thirteenth, fourteenth, fifteenth, sixteenth, or seventeenth embodiment.

Also provided herein are pharmaceutical compositions comprising 1) a compound having the Formula I:

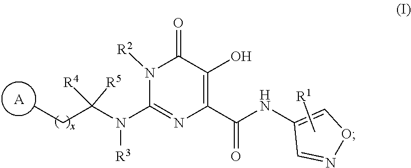

or a pharmaceutically acceptable salt thereof, wherein the variables are as described above for the first, second, third, fourth, fifth, sixth, seventh, eighth, ninth, tenth, eleventh, twelfth, thirteenth, fourteenth, fifteenth, sixteenth, seventeenth, or eighteenth embodiment; and 2) a pharmaceutically acceptable carrier.

Compounds having the Formula I are further disclosed in the Exemplification and are included in the present disclosure. Pharmaceutically acceptable salts thereof as well as the neutral forms are included.

### 4. Uses, Formulation and Administration

Compounds and compositions described herein are generally useful for modulating the activity of TREX1. In some aspects, the compounds and pharmaceutical compositions described herein inhibit the activity TREX1.

In some aspects, compounds and pharmaceutical compositions described herein are useful in treating a disorder associated with TREX1 function. Thus, provided herein are methods of treating a disorder associated with TREX1 function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with TREX1 function. Also provided is a compound described herein, or a pharmaceutically acceptable salt thereof, or a pharmaceutical composition comprising a disclosed compound or pharmaceutically acceptable salt thereof, for use in treating a disorder associated with TREX1.

In some aspects, the compounds and pharmaceutical compositions described herein are useful in treating cancer.

In some aspects, the cancer treated by the compounds and pharmaceutical compositions described herein is selected from colon cancer, gastric cancer, thyroid cancer, lung cancer, leukemia, pancreatic cancer, melanoma, multiple melanoma, brain cancer, CNS cancer, renal cancer, prostate cancer, ovarian cancer, leukemia, and breast cancer.

In some aspects, the cancer treated by the compounds and pharmaceutical compositions described herein is selected from lung cancer, breast cancer, pancreatic cancer, colorectal cancer, and melanoma.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Chemical Synthesis

The representative examples that follow are intended to help illustrate the present disclosure, and are not intended to, nor should they be construed to, limit the scope of the invention.

### Synthesis of 2-chloro-N-(isoxazol-4-yl)-5-methoxy-1-methyl-6-oxo-1,6-dihydropyrimidine-4-carboxamide (Int F)

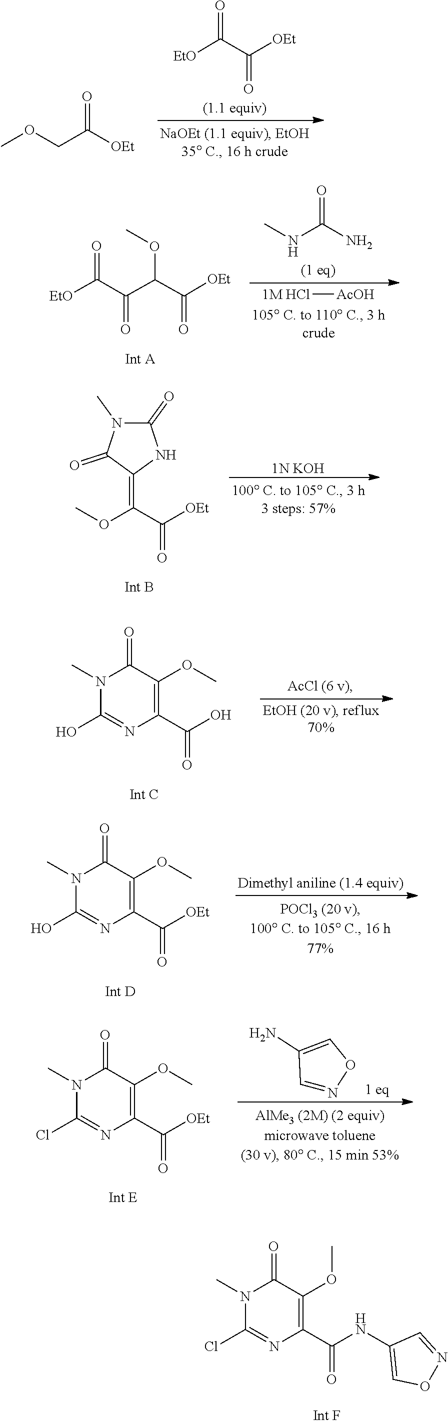

### Experimental Procedure

### Synthesis of 1,4-diethyl 2-methoxy-3-oxobutanedioate, Int A

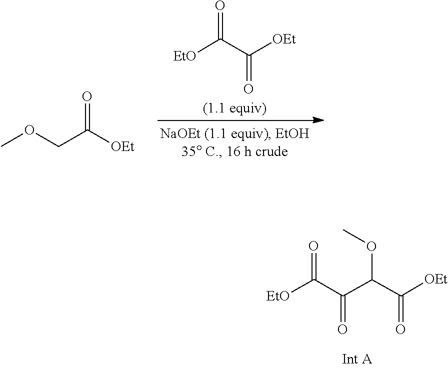

Into a 1 L 3-necked round-bottom flask purged and maintained with an inert atmosphere of nitrogen, was placed ethanol (600 mL), sodium ethanolate (31.7 g, 0.470 mmol, 1.10 equiv), ethyl oxalate (68.0 g, 467 mmol, 1.10 equiv) was added at room temperature. This was followed by the addition of ethyl 2-methoxyacetate (50.0 g, 423 mmol, 1.00 equiv) dropwise with stirring at room temperature. The resulting solution was stirred for 1 overnight at 35° C. The resulting mixture was concentrated under vacuum to remove most of ethanol. The pH value of the solution was adjusted to 3 with hydrogen chloride (1M) at 0° C. The resulting solution was extracted with 4×500 mL of ethyl acetate dried over anhydrous sodium sulfate and concentrated under vacuum. This resulted in 90 g (crude) of 1,4-diethyl 2-methoxy-3-oxobutanedioate (Int A) as brown oil.

### Synthesis of ethyl 2-methoxy-2-[(4E)-1-methyl-2,5-dioxoimidazolidin-4-ylidene]acetate, Int B

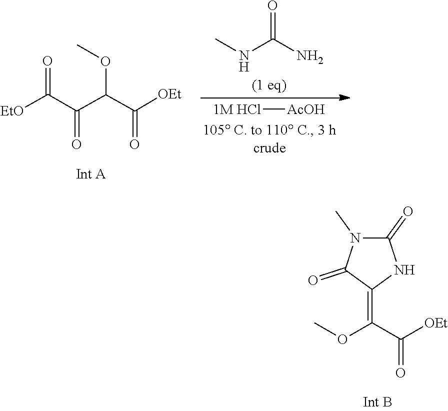

Into a 2 L round-bottom flask, was placed 1,4-diethyl 2-methoxy-3-oxobutanedioate (Int A) (90.0 g, 413 mmol, 1.00 equiv), methylurea (30.6 g, 413 mmol, 1.00 equiv), acetic acid (1.20 L), hydrogen chloride (400 mL, 4 M in dioxane). The resulting solution was stirred for 3 h at 105° C. The resulting mixture was concentrated under vacuum. The resulting mixture was washed with 1×500 ml of hexane. This resulted in 90 g (crude) of ethyl 2-methoxy-2-[(4E)-1-methyl-2,5-dioxoimidazolidin-4-ylidene]acetate (Int B) as a brown solid. 1H NMR (300 MHz, Chloroform-d) δ 8.75 (s, 1H), 7.47 (s, 0.4H), 5.09 (s, 2H), 4.51-4.30 (m, 3H), 3.85 (s, 1H), 3.84 (s, 3H), 3.12 (s, 3H), 3.07 (s, 1H), 2.14 (d, J=12.9 Hz, 1H), 1.45-1.42 (m, 2H), 1.42-1.37 (m, 3H).

### Synthesis of 2-hydroxy-5-methoxy-1-methyl-6-oxopyrimidine-4-carboxylic acid, Int C

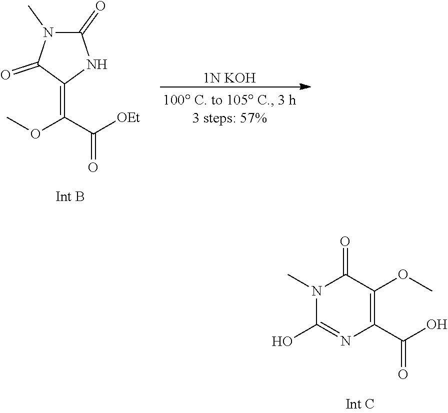 [SMILES: CCOC(=O)/C(OC)=C1\NC(=O)N(C)C1=O ; "1N KOH" ; COc1c(C(=O)O)nc(O)n(C)c1=O]

Into a 2 L round-bottom flask, was placed ethyl 2-methoxy-2-[(4E)-1-methyl-2,5-dioxoimidazolidin-4-ylidene]acetate (Int B) (80.0 g, 351 mmol, 1.00 equiv), potassium hydroxide (1M in water) (1.40 L). The resulting solution was stirred for 3 h at 105° C. The reaction mixture was cooled to 0° C. with a water/ice bath. The pH value of the solution was adjusted to 3 with hydrogen chloride (12 M) at 0° C., the solids were collected by filtration and the precipitate was dried in vacuo. This resulted in 40 g of 2-hydroxy-5-methoxy-1-methyl-6-oxopyrimidine-4-carboxylic acid (Int C) (yield 57%) as a white solid. ¹H NMR (300 MHz, DMSO-d6) δ 14.35 (s, 1H), 10.91 (s, 1H), 3.68 (s, 3H), 3.14 (s, 3H).

### Synthesis of ethyl 2-hydroxy-5-methoxy-1-methyl-6-oxopyrimidine-4-carboxylate, Int D

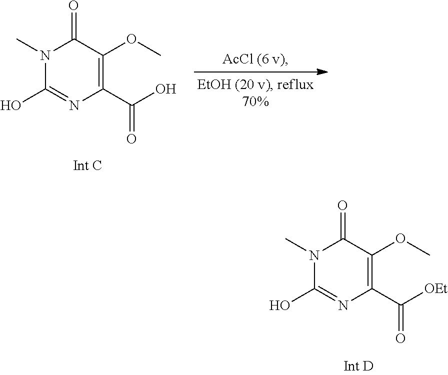 [SMILES: COc1c(C(=O)O)nc(O)n(C)c1=O ; CCOC(=O)c1nc(O)n(C)c(=O)c1OC]

Into a 1 L 3-necked round-bottom flask, purged and maintained with an inert atmosphere of argon, was placed 2-hydroxy-5-methoxy-1-methyl-6-oxopyrimidine-4-carboxylic acid (Int C) (20 g, 0.10 mmol, 1.00 equiv), and ethanol (400 mL). This was followed by the addition of acetyl chloride (118 g, 1.50 mmol, 15.0 equiv) dropwise with stirring at 0° C. The resulting solution was heated at reflux overnight. The reaction mixture was cooled with a water/ice bath. The solids were collected by filtration. This resulted in 16 g of ethyl 2-hydroxy-5-methoxy-1-methyl-6-oxopyrimidine-4-carboxylate (Int D) (yield: 70%) as a white solid. ¹H NMR (400 MHz, DMSO-d6) δ 11.08 (s, 1H), 4.32 (q, J=7.1 Hz, 2H), 3.70 (s, 3H), 3.15 (s, 3H), 1.31 (t, J=7.1 Hz, 3H).

### Synthesis of ethyl 2-chloro-5-methoxy-1-methyl-6-oxopyrimidine-4-carboxylate, Int E

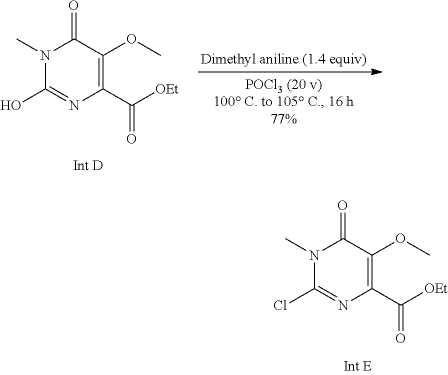 [SMILES: CCOC(=O)c1nc(O)n(C)c(=O)c1OC ; CCOC(=O)c1nc(Cl)n(C)c(=O)c1OC]

Into a 1-L 3-necked round-bottom flask purged and maintained with an inert atmosphere of argon, was placed ethyl 2-hydroxy-5-methoxy-1-methyl-6-oxopyrimidine-4-carboxylate (Int D) (16.0 g, 70.1 mmol, 1.00 equiv), dimethylaniline (1.20 g, 98.2 mmol, 1.40 equiv), and phosphoryl trichloride (320.0 mL). The resulting solution was stirred for 1 overnight at 100° C. The resulting mixture was concentrated under vacuum. The residue was applied onto a silica gel column and eluted with ethyl acetate/petroleum ether (1/10-1/4). This resulted in 13.3 g of ethyl 2-chloro-5-methoxy-1-methyl-6-oxopyrimidine-4-carboxylate (Int E) (yield 77.0%) as a yellow solid. ¹H NMR (300 MHz, DMSO-d6) δ 4.32 (q, J=7.1 Hz, 2H), 3.84 (s, 3H), 3.54 (s, 3H), 1.30 (t, J=7.1 Hz, 3H).

### Synthesis of 2-chloro-5-methoxy-1-methyl-N-(1, 2-oxazol-4-yl)-6-oxopyrimidine-4-carboxamide, Int F

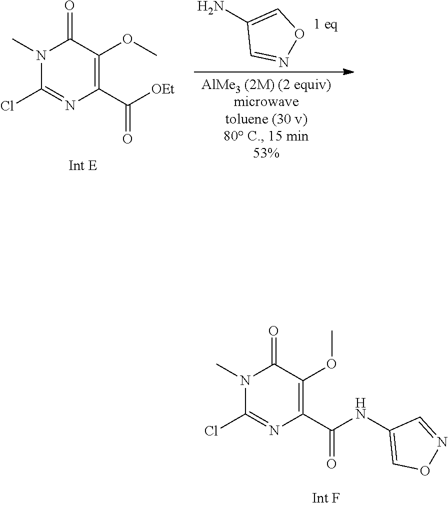 [SMILES: CCOC(=O)c1nc(Cl)n(C)c(=O)c1OC ; Nc1cnoc1 ; COc1c(C(=O)Nc2cnoc2)nc(Cl)n(C)c1=O]

To a stirred solution of ethyl 2-chloro-5-methoxy-1-methyl-6-oxopyrimidine-4-carboxylate (Int E) (1.00 g, 4.05 mmol, 1.00 equiv) and 1,2-oxazol-4-amine (341 mg, 4.05 mmol, 1.00 equiv) in toluene (15 mL) was added trimethylaluminum (2M in toluene) (4.1 mL, 8.10 mmol, 2.0 equiv) at room temperature under argon atmosphere. The resulting solution was stirred with microwave radiation for 15 min at 80° C. The reaction mixture was quenched with water/ice at 0° C. The resulting solution was extracted with 3×40 mL of ethyl acetate, the combined organic layers were washed with brine (1×30 mL), dried over anhydrous sodium sulfate and concentrated under reduced pressure. The residue was applied onto a silica gel column and eluted with ethyl acetate/petroleum ether (1/10-1/1). This resulted in 650 mg of 2-chloro-5-methoxy-1-methyl-N-(1, 2-oxazol-4-yl)-6-oxopyrimidine-4-carboxamide (Int F) (yield 53.0%) as a light yellow solid. ESI−MS m/z=285.2 [M+H]⁺. Calculated MW: 284.2 ¹H NMR (300 MHz, DMSO-d6) δ 10.84 (s, 1H), 9.28 (s, 1H), 8.78 (s, 1H), 3.86 (s, 3H), 3.62 (s, 3H).

### Synthesis of ([[2-(3-chlorophenyl)pyridin-3-yl]methyl](methyl)amine), Int H

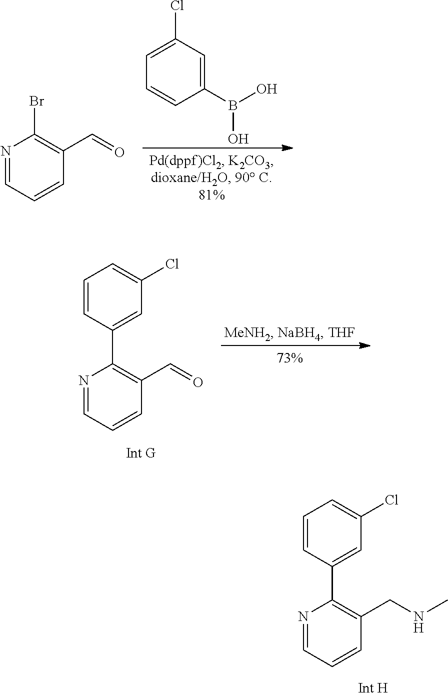 [SMILES: O=Cc1cccnc1Br ; OB(O)c1cccc(Cl)c1 ; O=Cc1cccnc1-c1cccc(Cl)c1 ; CNCc1cccnc1-c1cccc(Cl)c1]

### Synthesis of (2-(3-chlorophenyl) pyridine-3-carbaldehyde), Int G

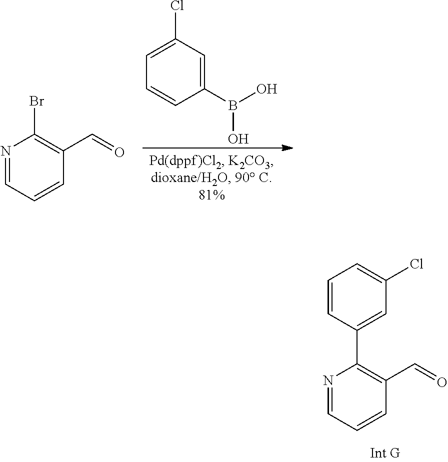 [SMILES: O=Cc1cccnc1Br ; OB(O)c1cccc(Cl)c1 ; O=Cc1cccnc1-c1cccc(Cl)c1]

Into a 100-mL 3-necked round-bottom flask, purged and maintained with an inert atmosphere of argon, was placed 2-bromopyridine-3-carbaldehyde (2.00 g, 10.7 mmol, 1.00 equiv), 3-chlorophenylboronic acid (2.52 g, 16.1 mmol, 1.50 equiv), Pd(dppf)C₁₂ (236 mg, 0.323 mmol, 0.0300 equiv), potassium carbonate (4.46 g, 32.3 mmol, 3.00 equiv), 1,4-dioxane (40.0 mL), and water (8.00 mL). The resulting solution was stirred for 3 h at 90° C. in an oil bath. The reaction was then quenched by the addition of 10 mL of water. The resulting solution was extracted with 3×30 mL of ethyl acetate dried over anhydrous sodium sulfate and concentrated. The residue was applied onto a silica gel column and eluted with ethyl acetate/petroleum ether (1:3). This resulted in 1.9 g of (2-(3-chlorophenyl) pyridine-3-carbaldehyde), Int G, (yield 81%) as a yellow solid. ESI−MS m/z=218.2 [M+H]⁺ Calculated MW: 217.0

The following intermediates were synthesized using similar conditions as those described in the step above along with appropriate starting materials.

| Structure | Intermediate | LCMS |
|---|---|---|
| 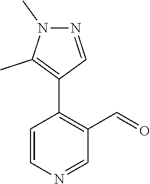 | Int G2 | ESI-MS m/z = 202.2 [M + H]+. Calculated MW: 201.09 |
| 4-(1,5-dimethyl- | | |
| 1H-pyrazol-4- | | |
| yl)nicotinaldehyde | | |

### Synthesis of ([[2-(3-chlorophenyl)pyridin-3-yl]methyl](methyl)amine, Int H

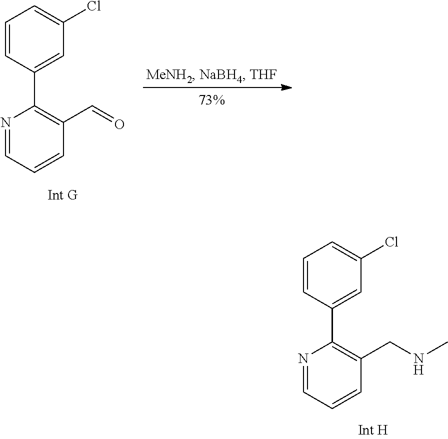 [SMILES: O=Cc1cccnc1-c1cccc(Cl)c1 ; CNCc1cccnc1-c1cccc(Cl)c1]

Into a 40-mL vial, was placed (2-(3-chlorophenyl) pyridine-3-carbaldehyde) Int G (900 mg, 4.13 mmol, 1.00 equiv), methanol (20.0 mL), acetic acid (0.100 mL), and methanamine (30% in methanol, 5.00 mL). The resulting solution was stirred for 2 h at room temperature. Sodium borohydride (313 mg, 8.27 mmol, 2.00 equiv) was added portion wise to the mixture at 0° C. The resulting solution was stirred for 12 h at 25° C. The reaction was then quenched by the addition of 5 mL of water. The resulting mixture was concentrated. The residue was applied onto a silica gel column and eluted with ethyl acetate/petroleum ether (1:3). This resulted in 700 mg of ([[2-(3-chlorophenyl)pyridin-3-yl]methyl](methyl)amine) Int H (yield 73%) as a yellow semi-solid. ESI−MS m/z=233.2 [M+H]+. Calculated MW: 232.1

The following intermediates were synthesized using similar conditions as those described in the step above along with appropriate starting materials.

| | Inter- | |
|---|---|---|
| Structure | mediate | LCMS |
| 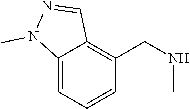 | Int H2 | ESI-MS m/z = 176.3 [M + H]+. Calculated MW: 175.11 |
| N-methyl-1-(1- | | |
| methyl-1H-indazol- | | |
| 4-yl)methanamine | | |
| 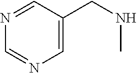 | Int H3 | ESI-MS m/z = 124.4 [M + H]+. Calculated MW: 123.1 |
| N-methyl-1- | | |
| (pyrimidin-5- | | |
| yl)methanamine | | |
| 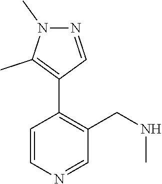 | Int H4 | ESI-MS m/z = 217.2 [M + H]+. Calculated MW: 216.14 |
| 1-(4-(1,5-dimethyl- | | |
| 1H-pyrazol-4- | | |
| yl)pyridin-3-yl)-N- | | |
| methylmethanamine | | |
| 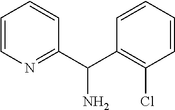 [SMILES: NC(c1ccccn1)c1ccccc1Cl] | Int H5 | ESI-MS m/z = 219.4 [M + H]+. Calculated MW: 218.1 |
| (2-chlorophenyl)(pyridin-2- | | |
| yl)methanamine | | |
| 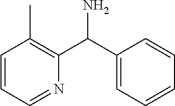 | Int H6 | ESI-MS m/z = 199.2 [M + H]+. Calculated MW: 198.2 |
| (3-methylpyridin-2- | | |
| yl)(phenyl)methanamine | | |
| 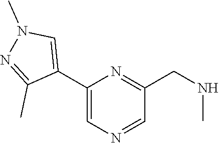 [SMILES: CNCc1cncc(-c2cn(C)nc2C)n1] | Int H7 | ESI-MS m/z = 218.2 [M + H]+. Calculated MW: 217.1 |
| 1-(6-(1,3-dimethyl-1H- | | |
| pyrazol-4-yl)pyrazin-2-yl)- | | |
| N-methylmethanamine | | |
| 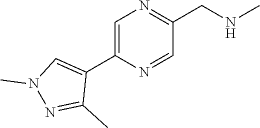 | Int H8 | ESI-MS m/z = 218.3 [M + H]+. Calculated MW: 217.1 |
| 1-(5-(1,3-dimethyl-1H- | | |
| pyrazol-4-yl)pyrazin-2-yl)- | | |
| N-methylmethanamine | | |

### Synthesis of (2-chlorophenyl)(phenyl)methanamine, Int K

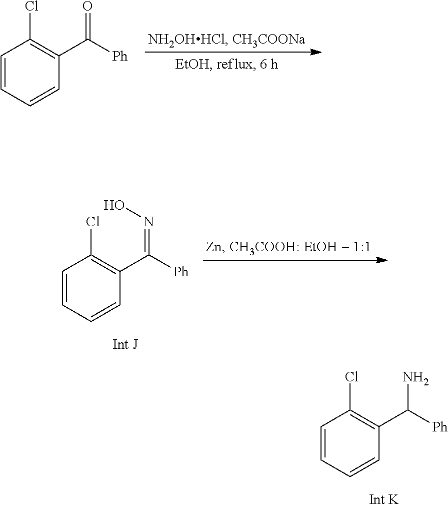 [SMILES: O=C(c1ccccc1)c1ccccc1Cl ; O/N=C(/c1ccccc1)c1ccccc1Cl ; NC(c1ccccc1)c1ccccc1Cl]

To a stirred solution of (2-chlorophenyl)(phenyl)methanone (25.0 g, 115 mmol, 1.00 equiv) and Hydroxylamine hydrochloride (12.0 g, 173 mmol, 1.50 equiv) were added sodium acetate (1.89 g, 231 mmol, 2.00 equiv) and ethyl alcohol (500 mL) in portions at room temperature under ambient atmosphere. The resulting mixture was stirred for 6 h at 80° C. under nitrogen atmosphere. The resulting mixture was concentrated under reduced pressure. The crude product ((Z)—N-[(2-chlorophenyl)(phenyl)methylidene]hydroxylamine), Int J was used in the next step directly without further purification.

To a stirred solution of Int J, ((Z)—N-[(2-chlorophenyl)(phenyl)methylidene]hydroxylamine) (50.0 g, 108 mmol, 1.00 equiv) and ethyl alcohol (250 mL) and acetic acid (250 mL) were added Zinc (70.6 g, 1080 mmol, 10.0 equiv) in portions at 0° C. under ambient atmosphere. The resulting mixture was stirred for 4 h at room temperature under nitrogen. The resulting mixture was filtered, the filter cake was washed with ethyl alcohol. The filtrate was concentrated under reduced pressure. The resulting mixture was diluted with water. The mixture was basified to pH 10 with sodium hydroxide, filtered, and the filter cake was washed with ethyl acetate. The resulting mixture was extracted with ethyl acetate, dried over anhydrous sodium sulfate. After filtration, the filtrate was concentrated under reduced pressure. The residue was purified by silica gel column chromatography, eluted with ethyl acetate/petroleum to afford 12.5 g of (1-(2-chlorophenyl)-1-phenylmethanamine) (yield 53%) as a light yellow solid. (ESI−MS m/z=201.2 [M−NH3]±. Calculated MW: 217.1)

The following intermediates were synthesized using similar conditions as those described in the steps above along with appropriate starting materials.

| | Inter- | |
|---|---|---|
| Structure | mediate | LCMS |
| 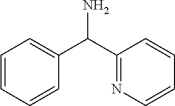 [SMILES: NC(c1ccccc1)c1ccccn1] | Int K2 | ESI-MS m/z = 185.1 [M + H]+. Calculated MW: 184.1 |
| phenyl(pyridin-2-yl)methanamine | | |
| 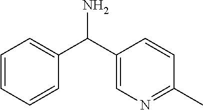 | Int K3 | ESI-MS m/z = 199.4 [M + H]+. Calculated MW: 198.1 |
| (6-methylpyridin-3-yl)(phenyl)methanamine | | |
| 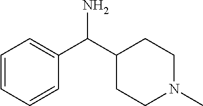 | Int K4 | ESI-MS m/z = 205.2 [M + H]+. Calculated MW: 204.1 |
| (1-methylpiperidin-4- | | |
| yl)(phenyl)methanamine | | |
| 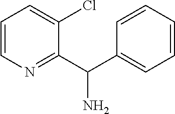 [SMILES: NC(c1ccccc1)c1ncccc1Cl] | Int K6 | ESI-MS m/z = 219.2 [M + H]+. Calculated MW: 218.1 |
| (3-chloropyridin-2- | | |
| yl)(phenyl)methanamine | | |
| 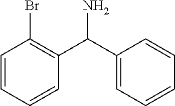 [SMILES: NC(c1ccccc1)c1ccccc1Br] | Int K14 | Commercial intermediate |
| (2-bromophenyl)(phenyl)methanamine | | |
| 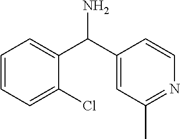 [SMILES: Cc1cc(C(N)c2ccccc2Cl)ccn1] | Int K16 | ESI-MS m/z = 233.1 [M + H]+. Calculated MW: 232.1 |
| (2-chlorophenyl)(2-methylpyridin-4- | | |
| yl)methanamine | | |
| 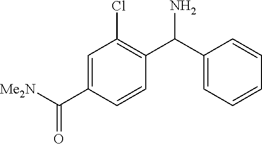 [SMILES: CN(C)C(=O)c1ccc(C(N)c2ccccc2)c(Cl)c1] | Int K17 | ESI-MS m/z = 289.3 [M + H]+. Calculated MW: 288.1 |
| 4-(amino(phenyl)methyl)-3- | | |
| chloro-N,N- | | |
| dimethylbenzamide | | |
| 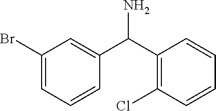 [SMILES: NC(c1cccc(Br)c1)c1ccccc1Cl] | Int K18 | ESI-MS m/z = 297.9 [M + H]+. Calculated MW: 297.0 |
| (3-bromophenyl)(2- | | |
| chlorophenyl)methanamine | | |

### Synthesis of 3-(amino(phenyl)methyl)benzonitrile, Int K5

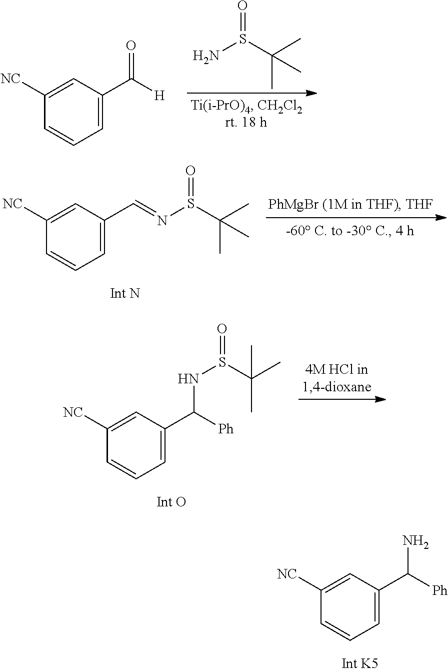

Into a 250-mL round-bottom flask, was placed (S)-2-methylpropane-2-sulfinamide (4.47 g, 36.9 mmol, 1.00 equiv), Ti(Oi-Pr)₄ (21 g, 74 mmol, 2.0 equiv), dichloromethane (90 mL). This was followed by the dropwise addition of a solution of 3-formylbenzonitrile (5.00 g, 38.0 mmol, 1.03 equiv) in dichloromethane (10 mL) at 0° C. The resulting solution was stirred for 18 h at room temperature. The reaction was then quenched by the addition of 50 mL of water. The solids were filtered out. The resulting solution was extracted with dichloromethane and the organic layers combined. The residue was applied onto a silica gel column with ethyl acetate/petroleum ether (2:3). This resulted in 5.5 g of (S)—N-[(3-cyanophenyl)methylidene]-2-methylpropane-2-sulfinamide, Int N (63% yield) as a white solid.

To a stirred solution of (S)—N-[(3-cyanophenyl)methylidene]-2-methylpropane-2-sulfinamide, Int N (1.0 g, 4.0 mmol, 1.0 equiv) in THF (15.0 mL) was added phenylmagnesium bromide (8.0 mL, 8.0 mmol, 2.0 equiv) dropwise at −70° C. under argon atmosphere. The resulting mixture was stirred for 2 h at −30° C. under argon. The reaction was quenched with saturated ammonium chloride at 0° C. The resulting mixture was extracted with ethyl acetate, washed with brine, dried over anhydrous sodium sulfate. After filtration, the filtrate was concentrated under reduced pressure. The residue was purified by silica gel column chromatography, eluted with petroleum ether/ethyl acetate (100:1-1:1) to afford 1 g of (S)—N-((3-cyanophenyl)(phenyl)methyl)-2-methylpropane-2-sulfinamide, Int 0 (yield, 68%) as a brown oil.

To a stirred solution of Int 0 ((S)—N-((3-cyanophenyl)(phenyl)methyl)-2-methylpropane-2-sulfinamide) (2.00 g, 6.09 mmol, 1.00 equiv) in hydrogen chloride (gas) in 1,4-dioxane (50.0 mL) was stirred at room temperature. The resulting mixture was stirred for overnight at room temperature. The precipitated solids were collected by filtration and washed with ethyl acetate. This resulted in 1.5 g of Int K5, 3-(amino(phenyl)methyl)benzonitrile hydrochloride (yield, 86%) as a yellow solid. (ESI−MS m/z=192.1 [M-NH3]±. Calculated MW: 208.1)

### Synthesis of (2,6-dichlorophenyl)(pyridin-2-yl)methanamine, Int K6

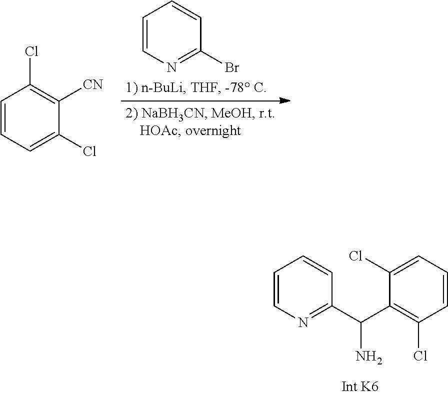 [SMILES: N#Cc1c(Cl)cccc1Cl ; Brc1ccccn1 ; NC(c1ccccn1)c1c(Cl)cccc1Cl]

Into a 250-mL 3-necked round-bottom flask, purged and maintained with an inert atmosphere of argon, were placed 2-bromopyridine (5.00 g, 31.6 mmol, 1.00 equiv) and tetrahydrofuran (100 mL). This was followed by the addition of n-butyllithium (2.5 M in hexane) (3.58 mL, 55.8 mmol, 1.20 equiv) dropwise with stirring at −78° C. To this was added 2, 6-dichlorobenzonitrile (7.08 g, 41.1 mmol, 1.30 equiv) dropwise with stirring at −78° C. The resulting solution was stirred for 2 h at −78° C. to −60° C. The reaction was quenched with saturated ammonium chloride at −20° C. The resulting mixture was extracted with ethyl acetate (3×50 mL). The combined organic layers were washed with water (3×50 mL), and dried over anhydrous sodium sulfate. After filtration, the filtrate was concentrated under reduced pressure. To the mixture was added methanol (100 mL) at 0° C., sodium cyanoborohydride (9.94 g, 158 mmol, 5.00 equiv), acetic acid (2.85 g, 47.5 mmol, 1.50 equiv). The resulting solution was stirred for 2 h at room temperature. The reaction was quenched with water at 0° C. The resulting mixture was extracted with ethyl acetate (3×50 mL). The combined organic layers were washed with water (1×100 mL), dried over anhydrous sodium sulfate. After filtration, the filtrate was concentrated under reduced pressure. This resulted in 3 g of Int K6 (1-(2,6-dichlorophenyl)-1-(pyridin-2-yl)methanamine) (yield 37%) as dark brown oil. (ESI−MS m/z=252.9 [M+H]+. Calculated MW: 252.0)

The following intermediates were synthesized using similar conditions as those described in the steps above along with appropriate starting materials.

| Structure | Intermediate | LCMS |
|---|---|---|
| 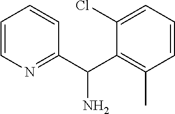 [SMILES: Cc1cccc(Cl)c1C(N)c1ccccn1] | Int K7 | ESI-MS m/z = 233.0 [M + H]+. Calculated MW: 232.1 |
| (2-chloro-6- | | |
| methylphenyl)(pyridin-2- | | |
| yl)methanamine | | |
| 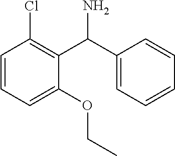 [SMILES: CCOc1cccc(Cl)c1C(N)c1ccccc1] | Int K8 | ESI-MS m/z = 245.1 [M + H − NH3]+. Calculated MW: 261.1 |
| (2-chloro-6-ethoxy- | | |
| phenyl)(phenyl) | | |
| methanamine | | |
| 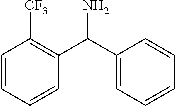 [SMILES: NC(c1ccccc1)c1ccccc1C(F)(F)F] | Int K13 | ESI-MS m/z = 252.2 [M + H]+. Calculated MW: 251.1 |
| phenyl(2- | | |
| (trifluoromethyl) | | |
| phenyl)methanamine | | |
| 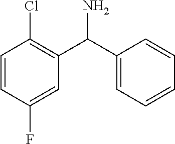 [SMILES: NC(c1ccccc1)c1cc(F)ccc1Cl] | Int K15 | ESI-MS m/z = 236.2 [M + H]+. Calculated MW: 235.1 |
| (2-chloro-5- | | |
| fluorophenyl)(phenyl) | | |
| methanamine | | |
| 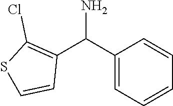 [SMILES: NC(c1ccccc1)c1ccsc1Cl] | Int K19 | ESI-MS m/z = 207.1 [M − NH₃]+. Calculated MW: 223.2 |
| (2-chlorothiophen-3- | | |
| yl)(phenyl)methanamine | | |

### Synthesis of N,N-dimethyl-2-((methylamino)methyl)benzamide, Int K9

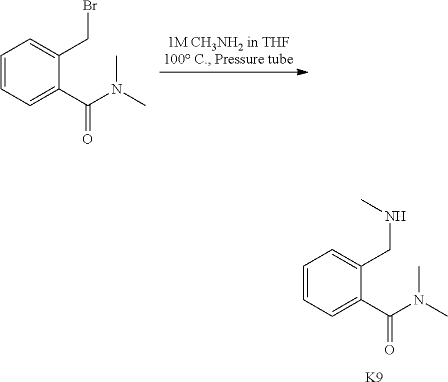

2-(Bromomethyl)-N,N-dimethylbenzamide (0.45 g, 1.9 mmol) and 1M solution of methylamine in THF (4.5 mL) were taken in a seal tube and stirred for 30 min at room temperature. Upon completion of the reaction (monitored by TLC), the solvent was evaporated to obtain crude title compound (0.4 g) which was used in the next step without further purification. ESI−MS m/z=193.0 [M+H]+. Calculated MW: 192.26

The following intermediates were synthesized using similar conditions as those described in the steps above along with appropriate starting materials.

| Structure | Intermediate | LCMS |
|---|---|---|
| 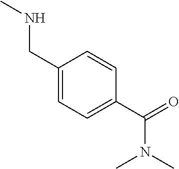 | Int K10 | ESI-MS m/z = 193.0 [M + H]+. Calculated MW: 192.26 |
| 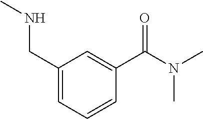 | Int K11 | ESI-MS m/z = 193.3 [M + H]+. Calculated MW: 192.26 |

### Synthesis of (2-chlorophenyl)(2-methylpyrimidin-5-yl)methanamine Int 0

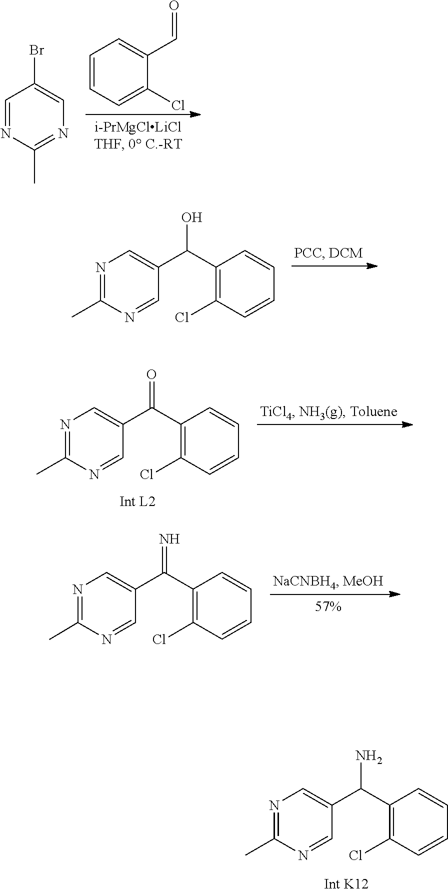

### Step-1: (2-Chlorophenyl)(2-methylpyrimidin-5-yl)Methanol

The iPrMgCl.LiCl (1.3M in THF) (180 mL, 234 mmol) was cooled to −78° C. under nitrogen atmosphere. To it, a solution of 5-bromo-2-methylpyrimidine (30 g, 170 mmol) in dry THF (150 mL) was added drop-wise at −78° C. The reaction mixture was stirred at −78° C. for 1.5 h. To this mixture, a solution of 2-chlorobenzaldehyde (31.6 g, 225 mmol) in dry THF (150 mL) was added drop wise at −78° C. The reaction mixture was warmed to room temperature and stirred at room temperature for 12 h. After completion of reaction (monitored by TLC), 10% ammonium chloride solution in water (1 L) was added slowly. The organic layer was separated, and the aqueous layer was extracted with ethyl acetate (2×1L). The combined organic layer was washed with brine (500 mL), dried over anhydrous sodium sulfate and concentrated under reduced pressure to get crude compound. The crude compound was purified by column chromatography (n-hexanes:ethyl acetate) to obtain title compound (8.5 g, 20%). LCMS: ESI−MS m/z=235.16 [M+H]+. Calculated MW: 234.68

### Step-2: (2-Chlorophenyl)(2-methylpyrimidin-5-yl)methanone

To a stirred solution of (2-chlorophenyl)(2-methylpyrimidin-5-yl)methanol (8 g, 34 mmol) in dry dichloromethane (160 mL) under an atmosphere of Nitrogen was lot-wise added pyridinium chlorochromate (8.13 g, 37.7 mmol) at room temperature. The resulting reaction mixture was stirred for another 12 h. After completion of reaction (monitored by TLC), the reaction mixture was filtered through celite-bed, washed with ethyl acetate (3×100 mL) and filtrate was then concentrated under reduced pressure to obtain crude compound which was purified by using column chromatography (n-hexanes:ethyl acetate) to give the title compound (5 g, 63%). LCMS: ESI−MS m/z=233.16 [M+H]+. Calculated MW: 232.67

### Step-3: (2-Chlorophenyl)(2-methylpyrimidin-5-yl)methanimine

To a stirred solution of (2-chlorophenyl)(2-methylpyrimidin-5-yl)methanone (0.650 g, 2.79 mmol) in toluene (6.5 mL) was added TiCl₄ (0.742 g, 3.91 mmol) dropwise at −78° C. The reaction mixture was stirred for 15 minutes. To the reaction, NH3 (g) was purged at −78° C. and reaction was stirred at room temperature overnight. Upon completion of reaction (monitored by TLC), the reaction mixture was filtered through celite-bed, washed with ethyl acetate (3×30 mL). The combined filtrate was concentrated under reduced pressure. The obtained crude title compound was used in the next step without further purification (0.5 g). LCMS: ESI−MS m/z=232.10 [M+H]+. Calculated MW: 231.68

### Step-4: (2-Chlorophenyl)(2-methylpyrimidin-5-yl)methanamine, Int K12

To a stirred solution of (2-chlorophenyl)(2-methylpyrimidin-5-yl)methanimine (0.5 g, 2 mmol) in methanol (5 mL), acetic acid (0.2 g) was added and reaction mixture was stirred for 15 minutes. To it, sodium cyanoborohydride (0.204 g, 3.24 mmol) was added and reaction mixture was stirred at room temperature for another 3 h. Upon completion of reaction (monitored by TLC), the reaction mixture was diluted in ethyl acetate (2×30 mL) and washed with saturated sodium bicarbonate solution (3×20 mL) followed by the brine (20 mL). The organic layer was separated, dried over sodium sulfate and evaporated to dryness to afford crude compound. The crude compound obtained was purified by column chromatography using basic alumina in dichloromethane:MeOH to obtain pure title compound (0.30 g, 46% (2 steps)). LCMS: ESI−MS m/z=234.10 [M+H]+. Calculated MW: 233.70.

### Synthesis of 4-benzoyl-3-chloro-N,N-dimethylbenzamide, Int L2

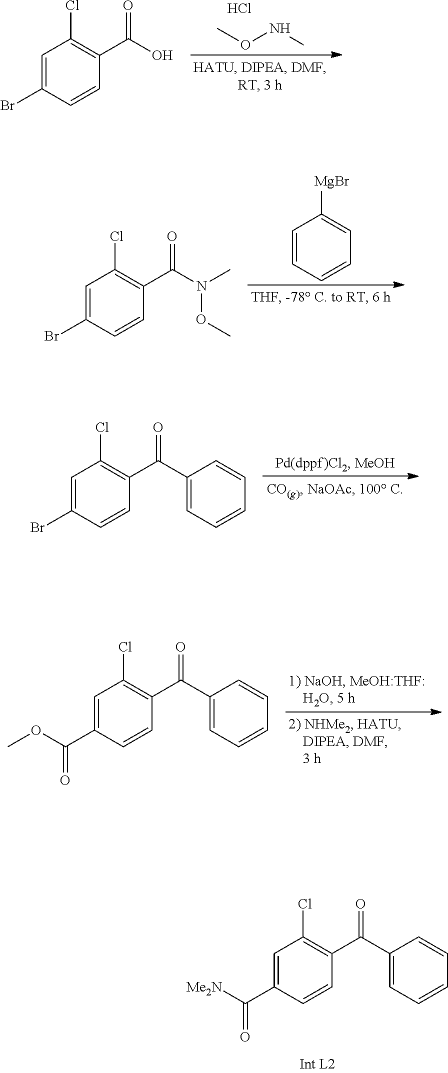

### Step-1: 4-bromo-2-chloro-N-methoxy-N-methylbenzamide

To a stirred solution of 4-bromo-2-chlorobenzoic acid (5.0 g, 21.23 mmol) in dry DMF (50 mL) was added N,O-dimethyl hydroxylamine hydrochloride (2.48 g, 25.5 mmol) followed by the addition of HATU (12.10 g, 31.85 mmol) at 0° C. The reaction mixture was stirred at 0° C. for 1 h. To the mixture, DIPEA (10.95 mL, 63.70 mmol) was added drop-wise at 0° C. and the resulting reaction mixture was stirred at room temperature for 4 h. Upon completion of reaction (monitored by TLC), water (250 ml) was added slowly and extracted with ethyl acetate (2×100 mL). The combined organic layer was washed with brine (200 mL), dried over anhydrous sodium sulfate and concentrated under reduced pressure. The crude compound was purified by column chromatography to give pure title compound (5.0 g, 84%). LCMS: ESI−MS m/z=280.1 [M+2H]+. Calculated MW: 278.53

### Step-2: (4-bromo-2-chlorophenyl)(phenyl)methanone

To a stirred solution of 4-bromo-2-chloro-N-methoxy-N-methylbenzamide (5.1 g, 18.31 mmol) in dry THF (50 mL) was added phenylmagnesium bromide (27.5 mL, 1M in THF, 27.5 mmol) at −78° C. Reaction mixture was allowed to come at room temperature and stirred for 16 h. Upon completion of reaction (monitored by TLC), saturated ammonium chloride (100 mL) was added slowly and reaction mixture was extracted with ethyl acetate (2×100 mL). The combined organic layer was washed with brine (100 mL), dried over anhydrous sodium sulfate and concentrated under reduced pressure. The crude compound was purified by column chromatography to give pure title compound (3.53 g, 65%). LCMS: ESI−MS m/z=297.3 [M+2H]+. Calculated MW: 295.5.

### Step-3: Methyl 4-benzoyl-3-chlorobenzoate

A solution of (4-bromo-2-chlorophenyl)(phenyl)methanone (3.0 g, 10 mmol) in MeOH (60 mL) was taken in steel pressure reactor under nitrogen atmosphere. To this, sodium acetate (2.41 g, 29.4 mmol), Pd(OAc)₂ (0.227 g, 1.01 mmol) and PdCl₂(dppf) (0.741 g, 1.01 mmol) were added. The vessel was filled with CO gas to about 150 PSI pressure and reaction mixture was stirred at room temperature for 16 h. Upon completion of reaction (monitored by TLC), the reaction mixture was filtered through celite-bed and washed with methanol (2×60 mL). The filtrate was concentrated, and crude compound was purified by column chromatography to give pure title compound (1.8 g, 64%). LCMS: ESI−MS m/z=275.1 [M+H]+. Calculated MW: 274.70

### Step-4: 4-benzoyl-3-chloro-N,N-dimethylbenzamide

To a stirred solution of methyl 4-benzoyl-3-chlorobenzoate (1.8 g, 6.55 mmol) in Methanol:THF:Water (1:1:1, 48 mL) was added sodium hydroxide (0.314 g, 7.86 mmol) at room temperature. The reaction mixture was stirred at room temperature for 3 h. Upon completion of reaction (monitored by TLC), the reaction mixture was concentrated under reduced pressure and azeotrope with dichloromethane (3×10 mL). Reaction mixture was dried under high vacuum to get the sodium salt of 4-benzoyl-3-chlorobenzoate (1.8 g crude, 97%). LCMS: ESI−MS m/z=259.1 [M−H]+. Calculated MW: 260.67

To a stirred solution of above prepared sodium salt of 4-benzoyl-3-chlorobenzoate (1.8 g, 6.4 mmol) in dry DMF (20 mL) was added HATU (3.63 g, 9.55 mmol) at room temperature. The reaction mixture was stirred at room temperature for 1 h. To the above solution, DIPEA (3.29 mL, 19.1 mmol) was added drop-wise followed by the addition of dimethylamine (0.52 mL, 9.55 mmol) at room temperature. The reaction mixture was allowed to stir at room temperature for 4 h. Upon completion of reaction (monitor by TLC), the reaction mixture was diluted with water (100 mL) and the aqueous layer was extracted with ethyl acetate (2×100 mL). The combined organic layer was washed with brine (50 mL), dried over anhydrous sodium sulfate and concentrated under reduced pressure. The crude product was purified by Column chromatography to give title compound (0.60 g, 31%). LCMS: ESI−MS m/z=288.3 [M+H]+. Calculated MW: 287.74

### General Procedure

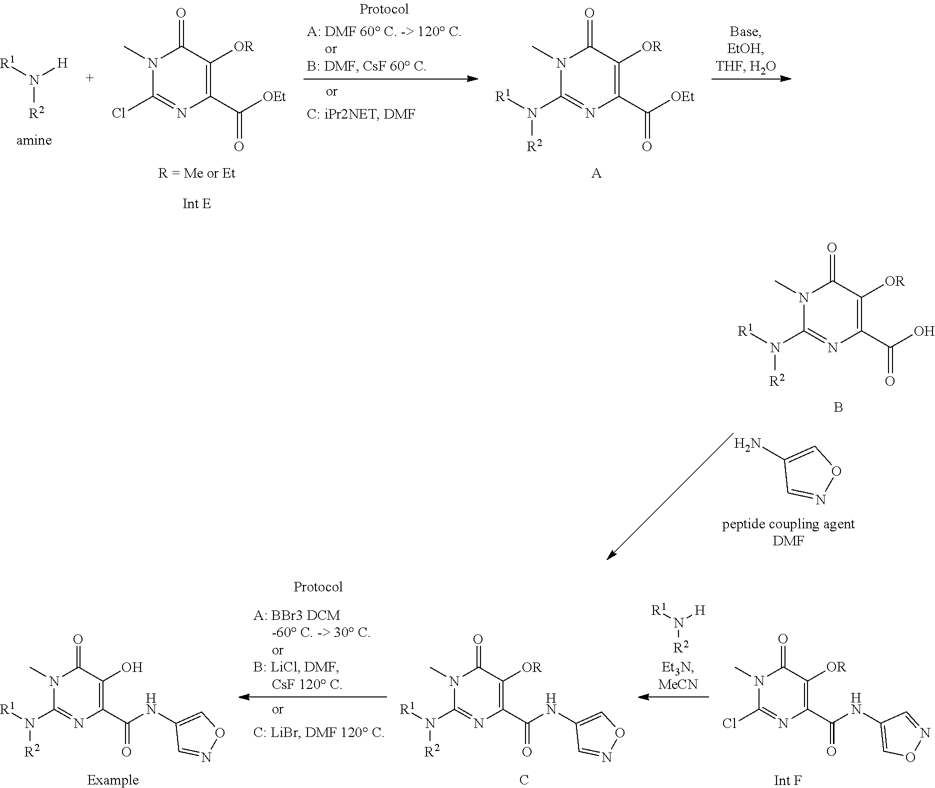

### Synthesis of ethyl 2-(dibenzylamino)-5-ethoxy-1-methyl-6-oxo-1,6-dihydropyrimidine-4-carboxylate, 1

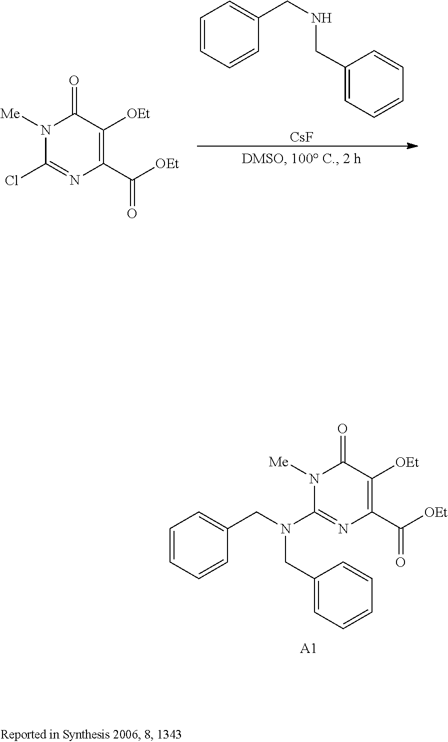

Ethyl 2-chloro-5-ethoxy-1-methyl-6-oxo-1,6-dihydropyrimidine-4-carboxylate (130 mg, 498 μmol), cesium fluoride (76 mg, 498 μmol), and dibenzylamine (196 mg, 996 μmol) were dissolved in DMSO (498 μL) at room temperature and the reaction was stirred at 100° C. for 2 h. The crude reaction mixture was directly purified on a 10 g reverse phase column to give 130 mg of ethyl 2-(dibenzylamino)-5-ethoxy-1-methyl-6-oxo-1,6-dihydropyrimidine-4-carboxylate, A1 in 62.2% yield. Calculated MW: 421.497; ESI−MS m/z=422.2 [M+H]+.

The following intermediates were synthesized using similar conditions as those described in the step above along with appropriate starting materials.

| Structure | Intermediate | LCMS |
|---|---|---|
| 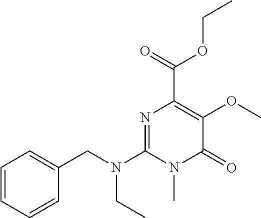 [SMILES: CCOC(=O)c1nc(N(CC)Cc2ccccc2)n(C)c(=O)c1OC] | A2 | ESI-MS m/z = 346.2 [M + H]+. Calculated MW: 345.1 |
| ethyl 2-(benzyl(ethyl)amino)-5-methoxy-1-methyl-6- | | |
| oxo-1,6-dihydropyrimidine-4-carboxylate | | |
| 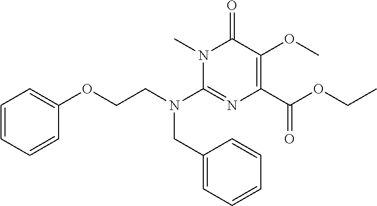 [SMILES: CCOC(=O)c1nc(N(CCOc2ccccc2)Cc2ccccc2)n(C)c(=O)c1OC] | A3 | ESI-MS m/z = 438.4 [M + H]+. Calculated MW: 437.2 |
| ethyl 2-(benzyl(2- | | |
| phenoxyethyl)amino)-5-methoxy- | | |
| 1-methyl-6-oxo-1,6- | | |
| dihydropyrimidine-4-carboxylate | | |
| 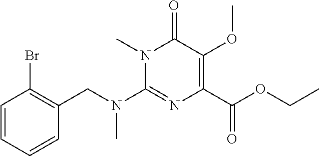 [SMILES: CCOC(=O)c1nc(N(C)Cc2ccccc2Br)n(C)c(=O)c1OC] | A4 | ESI-MS m/z = 410.1 [M + H]+. Calculated MW: 409.1 |
| ethyl 2-((2-bromobenzyl)(methyl)amino)-5- | | |
| methoxy-1-methyl-6-oxo-1,6- | | |
| dihydropyrimidine-4-carboxylate | | |
| 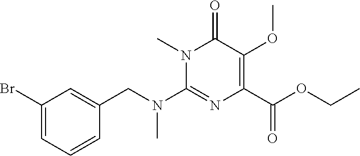 [SMILES: CCOC(=O)c1nc(N(C)Cc2cccc(Br)c2)n(C)c(=O)c1OC] | A5 | ESI-MS m/z = 410.1 [M + H]+. Calculated MW: 409.1 |
| ethyl 2-((3- | | |
| bromobenzyl)(methyl)amino)-5- | | |
| methoxy-1-methyl-6-oxo-1,6- | | |
| dihydropyrimidine-4-carboxylate | | |
| 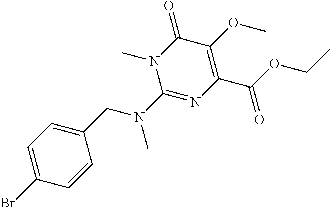 [SMILES: CCOC(=O)c1nc(N(C)Cc2ccc(Br)cc2)n(C)c(=O)c1OC] | A24 | ESI-MS m/z = 410.0/412.0 [M + H]+. Calculated MW: 409.1 |
| ethyl 2-((4-bromobenzyl)(methyl)amino)- | | |
| 5-methoxy-1-methyl-6-oxo-1,6- | | |
| dihydropyrimidine-4-carboxylate | | |
| 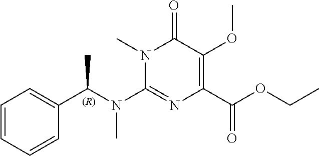 | A26 | ESI-MS m/z = 346.2 [M + H]+. Calculated MW: 345.2 |
| ethyl (R)-5-methoxy-1-methyl-2- | | |
| (methyl(1-phenylethyl)amino)-6- | | |
| oxo-1,6-dihydropyrimidine-4- | | |
| carboxylate | | |
| 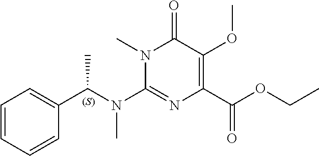 | A27 | ESI-MS m/z = 346.2 [M + H]+. Calculated MW: 345.2 |
| ethyl (S)-5-methoxy-1-methyl-2- | | |
| (methyl(1-phenylethyl)amino)-6- | | |
| oxo-1,6-dihydropyrimidine-4- | | |
| carboxylate | | |
| 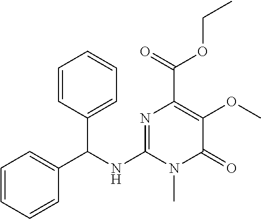 [SMILES: CCOC(=O)c1nc(NC(c2ccccc2)c2ccccc2)n(C)c(=O)c1OC] | A29 | ESI-MS m/Z = 394.2 [M + H]+. Calculated MW: 393.2 |
| ethyl 2-(benzhydrylamino)-5- | | |
| methoxy-1-methyl-6-oxo-1,6- | | |
| dihydropyrimidine-4-carboxylate | | |
| 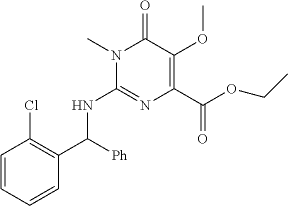 [SMILES: CCOC(=O)c1nc(NC(c2ccccc2)c2ccccc2Cl)n(C)c(=O)c1OC] | A30 | ESI-MS m/z = 428.4 [M + H]+. Calculated MW: 427.1 |
| ethyl 2-(((2- | | |
| chlorophenyl)(phenyl) | | |
| methyl)amino)-5-methoxy-1- | | |
| methyl-6-oxo-1,6- | | |
| dihydropyrimidine-4- | | |
| carboxylate | | |
| 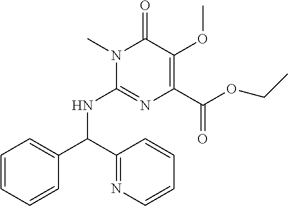 [SMILES: CCOC(=O)c1nc(NC(c2ccccc2)c2ccccn2)n(C)c(=O)c1OC] | A31 | ESI-MS m/z = 395.2 [M + H]+. Calculated MW: 394.2 |
| ethyl 5-methoxy-1- | | |
| methyl-6-oxo-2- | | |
| ((phenyl(pyridin-2- | | |
| yl)methyl)amino)-1,6- | | |
| dihydropyrimidine- | | |
| 4-carboxylate | | |
| 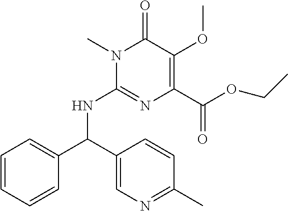 [SMILES: CCOC(=O)c1nc(NC(c2ccccc2)c2ccc(C)nc2)n(C)c(=O)c1OC] | A32 | ESI-MS m/z = 409.2 [M + H]+. Calculated MW: 408.2 |
| ethyl 5-methoxy-1- | | |
| methyl-2-(((6- | | |
| methylpyridin-3- | | |
| yl)(phenyl)methyl) | | |
| amino)-6-oxo-1,6- | | |
| dihydropyrimidine- | | |
| 4-carboxylate | | |
| 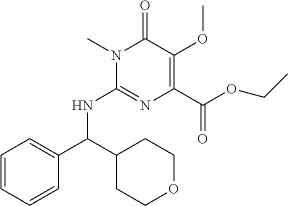 [SMILES: CCOC(=O)c1nc(NC(c2ccccc2)C2CCOCC2)n(C)c(=O)c1OC] | A33 | ESI-MS m/z = 402.4 [M + H]+. Calculated MW: 401.2 |
| ethyl 5-methoxy-1-methyl-6- | | |
| oxo-2-((phenyl(tetrahydro- | | |
| 2H-pyran-4-yl)methyl)amino)- | | |
| 1,6-dihydropyrimidine-4- | | |
| carboxylate | | |
| 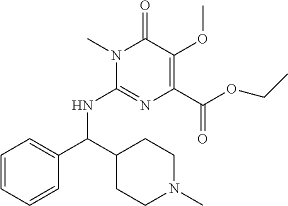 [SMILES: CCOC(=O)c1nc(NC(c2ccccc2)C2CCN(C)CC2)n(C)c(=O)c1OC] | A35 | ESI-MS m/z = 415.2 [M + H]+. Calculated MW: 414.2 |
| ethyl 5-methoxy-1-methyl-2-(((1- | | |
| methylpiperidin-4- | | |
| yl)(phenyl)methyl)amino)-6-oxo- | | |
| 1,6-dihydropyrimidine-4- | | |
| carboxylate | | |
| 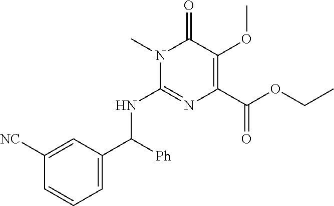 [SMILES: CCOC(=O)c1nc(NC(c2ccccc2)c2cccc(C#N)c2)n(C)c(=O)c1OC] | A36 | ESI-MS m/z = 419.1 [M + H]+. Calculated MW: 418.2 |
| ethyl 2-(((3-cyanophenyl)(phenyl)methyl)amino)- | | |
| 5-methoxy-1-methyl-6-oxo-1,6- | | |
| dihydropyrimidine-4-carboxylate | | |
| 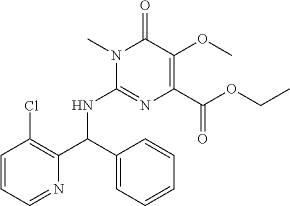 [SMILES: CCOC(=O)c1nc(NC(c2ccccc2)c2ncccc2Cl)n(C)c(=O)c1OC] | A38 | ESI-MS m/z = 429.1 [M + H]+. Calculated MW: 428.1 |
| ethyl 2-(((3-chloropyridin-2- | | |
| yl)(phenyl)methyl)amino)-5- | | |
| methoxy-1-methyl-6-oxo-1,6- | | |
| dihydropyrimidine-4-carboxylate | | |
| 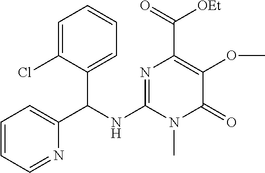 [SMILES: CCOC(=O)c1nc(NC(c2ccccn2)c2ccccc2Cl)n(C)c(=O)c1OC] | A40 | ESI-MS m/z = 429.4 [M + H]+. Calculated MW: 428.1 |
| ethyl 2-(((2-chlorophenyl)(pyridin-2- | | |
| yl)methyl)amino)-5-methoxy-1-methyl- | | |
| 6-oxo-1,6-dihydropyrimidine-4- | | |
| carboxylate | | |
| 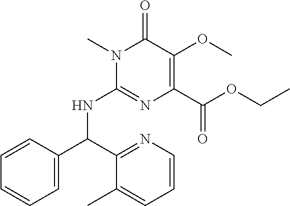 [SMILES: CCOC(=O)c1nc(NC(c2ccccc2)c2ncccc2C)n(C)c(=O)c1OC] | A41 | ESI-MS m/z = 409.4 [M + H]⁺. Calculated MW: 408.2 |
| ethyl 5-methoxy-1-methyl-2-(((3- | | |
| methylpyridin-2-yl)(phenyl)methyl)amino)- | | |
| 6-oxo-1,6-dihydropyrimidine-4-carboxylate | | |
| 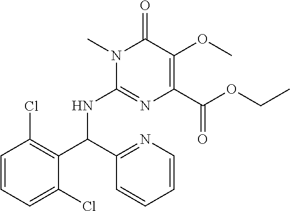 [SMILES: CCOC(=O)c1nc(NC(c2ccccn2)c2c(Cl)cccc2Cl)n(C)c(=O)c1OC] | A42 | ESI-MS m/z = 463.1 [M + H]+. Calculated MW: 462.1 |
| ethyl 2-(((2,6-dichlorophenyl)(pyridin-2- | | |
| yl)methyl)amino)-5-methoxy-1-methyl-6- | | |
| oxo-1,6-dihydropyrimidine-4-carboxylate | | |
| 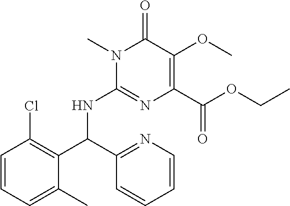 [SMILES: CCOC(=O)c1nc(NC(c2ccccn2)c2c(C)cccc2Cl)n(C)c(=O)c1OC] | A43 | ESI-MS m/z = 443.3 [M + H]+. Calculated MW: 442.1 |
| ethyl 2-(((2-chloro-6- | | |
| methylphenyl)(pyridin-2-yl)methyl)amino)- | | |
| 5-methoxy-1-methyl-6-oxo-1,6- | | |
| dihydropyrimidine-4-carboxylate | | |
| 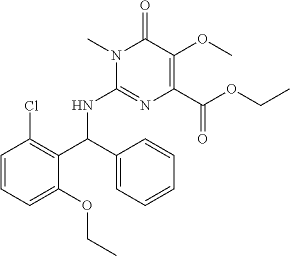 [SMILES: CCOC(=O)c1nc(NC(c2ccccc2)c2c(Cl)cccc2OCC)n(C)c(=O)c1OC] | A44 | ESI-MS m/z = 472.4 [M + H]+. Calculated MW: 471.2 |
| ethyl 2-(((2-chloro-6- | | |
| ethoxyphenyl)(phenyl)methyl)amino)- | | |
| 5-methoxy-1-methyl-6-oxo-1,6- | | |
| dihydropyrimidine-4-carboxylate | | |
| 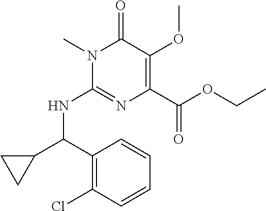 [SMILES: CCOC(=O)c1nc(NC(c2ccccc2Cl)C2CC2)n(C)c(=O)c1OC] | A45 | ESI-MS m/z = 392.4 [M + H]+. Calculated MW: 391.1 |
| ethyl 2-(((2- | | |
| chlorophenyl)(cyclopropyl)methyl) | | |
| amino)-5-methoxy-1-methyl-6- | | |
| oxo-1,6-dihydropyrimidine-4- | | |
| carboxylate | | |
| 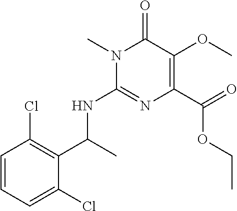 [SMILES: CCOC(=O)c1nc(NC(C)c2c(Cl)cccc2Cl)n(C)c(=O)c1OC] | A46 | ESI-MS m/z = 400.1 [M + H]+. Calculated MW: 399.1 |
| ethyl 2-((1-(2,6- | | |
| dichlorophenyl)ethyl)amino)- | | |
| 5-methoxy-1-methyl-6- | | |
| oxo-1,6-dihydropyrimidine- | | |
| 4-carboxylate | | |
| 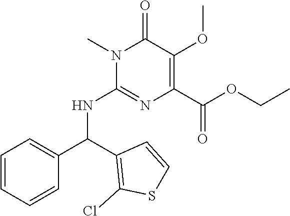 [SMILES: CCOC(=O)c1nc(NC(c2ccccc2)c2ccsc2Cl)n(C)c(=O)c1OC] | A62 | ESI-MS m/z = 433.9 [M + H]+. Calculated MW: 433.1 |
| ethyl 2-(((2-chlorothiophen-3- | | |
| yl)(phenyl)methyl)amino)-5- | | |
| methoxy-1-methyl-6-oxo-1,6- | | |
| dihydropyrimidine-4-carboxylate | | |

### Synthesis of Ethyl 2-(benzyl (methyl)amino)-5-ethoxy-1-methyl-6-oxo-1,6-dihydropyrimidine-4-carboxylate, A6

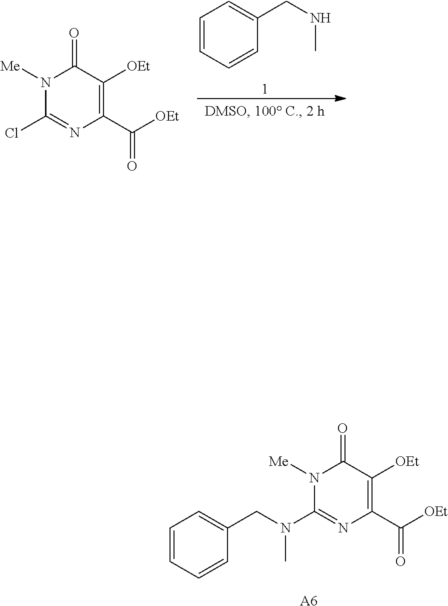

A mixture of Ethyl 2-chloro-5-ethoxy-1-methyl-6-oxo-1,6-dihydropyrimidine-4-carboxylate (0.400 g, 1.53 mmol), N-methyl-1-phenylmethanamine (0.371 g, 3.06 mmol) in dry DMSO (4 ml) was heated at 110° C. for 3 h. The reaction mixture was cooled to room temperature and poured into mixture of ice-cold water (50 mL). Reaction mixture was extracted with Ethyl acetate (2×100 mL). The combined organic layer was dried over anhydrous Na₂SO₄ and concentrated under reduced pressure. The crude compound was purified by flash chromatography to give the pure title compound (0.30 g, 56%). Calculated MW: 345.4. ESI−MS m/z=346.2 [M+H]⁺.

The following intermediates were synthesized using similar conditions as those described in the step above along with appropriate starting materials.

| Structure | Intermediate | LCMS |
|---|---|---|
| 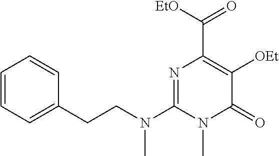 [SMILES: CCOC(=O)c1nc(N(C)CCc2ccccc2)n(C)c(=O)c1OCC] | A7 | ESI-MS m/z = 360.4 [M + H]+. Calculated MW: 359.43 |
| ethyl 5-ethoxy-1-methyl-2- | | |
| (methyl(phenethyl)amino)-6-oxo- | | |
| 1,6-dihydropyrimidine-4-carboxylate | | |
| 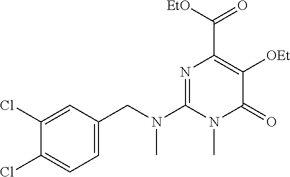 [SMILES: CCOC(=O)c1nc(N(C)Cc2ccc(Cl)c(Cl)c2)n(C)c(=O)c1OCC] | A8 | ESI-MS m/z = 414.5 [M + H]+. Calculated MW: 413.1 |
| ethyl 2-((3,4- | | |
| dichlorobenzyl)(methyl)amino)- | | |
| 5-ethoxy-1-methyl-6-oxo-1,6- | | |
| dihydropyrimidine-4-carboxylate | | |
| 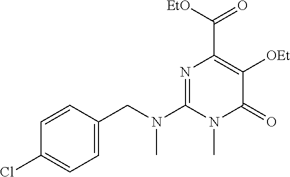 [SMILES: CCOC(=O)c1nc(N(C)Cc2ccc(Cl)cc2)n(C)c(=O)c1OCC] | A9 | ESI-MS m/z = 380.5 [M + H]+. Calculated MW: 379.84 |
| ethyl 2-((4- | | |
| chlorobenzyl)(methyl)amino)-5- | | |
| ethoxy-1-methyl-6-oxo-1,6- | | |
| dihydropyrimidine-4-carboxylate | | |
| 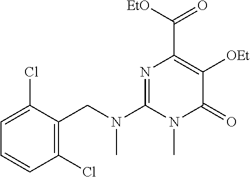 [SMILES: CCOC(=O)c1nc(N(C)Cc2c(Cl)cccc2Cl)n(C)c(=O)c1OCC] | A10 | ESI-MS m/z = 414.5 [M + H]+. Calculated MW: 413.1 |
| ethyl 2-((2,6- | | |
| dichlorobenzyl)(methyl)amino)-5- | | |
| ethoxy-1-methyl-6-oxo-1,6- | | |
| dihydropyrimidine-4-carboxylate | | |
| 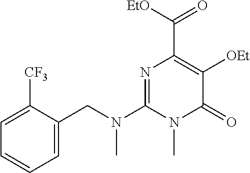 [SMILES: CCOC(=O)c1nc(N(C)Cc2ccccc2C(F)(F)F)n(C)c(=O)c1OCC] | A11 | ESI-MS m/z = 414.6 [M + H]+. Calculated MW: 413.4 |
| ethyl 5-ethoxy-1-methyl-2-(methyl(2- | | |
| (trifluoromethyl)benzyl)amino)-6-oxo- | | |
| 1,6-dihydropyrimidine-4-carboxylate | | |
| 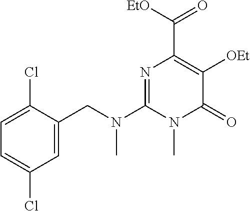 [SMILES: CCOC(=O)c1nc(N(C)Cc2cc(Cl)ccc2Cl)n(C)c(=O)c1OCC] | A12 | ESI-MS m/z = 414.5 [M + H]+. Calculated MW: 414.3 |
| ethyl 2-((2,5- | | |
| dichlorobenzyl)(methyl)amino)-5- | | |
| ethoxy-1-methyl-6-oxo-1,6- | | |
| dihydropyrimidine-4-carboxylate | | |
| 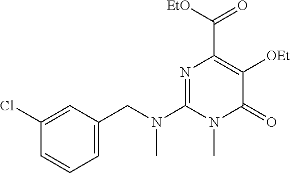 [SMILES: CCOC(=O)c1nc(N(C)Cc2cccc(Cl)c2)n(C)c(=O)c1OCC] | A13 | ESI-MS m/z = 380.4 [M + H]+. Calculated MW: 379.84 |
| ethyl 2-((3- | | |
| chlorobenzyl)(methyl)amino)-5- | | |
| ethoxy-1-methyl-6-oxo-1,6- | | |
| dihydropyrimidine-4-carboxylate | | |
| 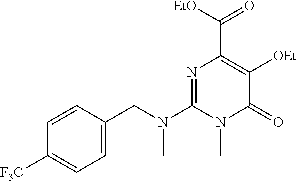 [SMILES: CCOC(=O)c1nc(N(C)Cc2ccc(C(F)(F)F)cc2)n(C)c(=O)c1OCC] | A14 | ESI-MS m/z = 414.5 [M + H]+. Calculated MW: 413.4 |
| ethyl 5-ethoxy-1-methyl-2-(methyl(4- | | |
| (trifluoromethyl)benzyl)amino)-6-oxo-1,6- | | |
| dihydropyrimidine-4-carboxylate | | |
| 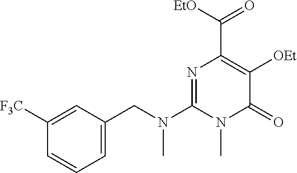 [SMILES: CCOC(=O)c1nc(N(C)Cc2cccc(C(F)(F)F)c2)n(C)c(=O)c1OCC] | A15 | ESI-MS m/z = 414.6 [M + H]+. Calculated MW: 413.4 |
| ethyl 5-ethoxy-1-methyl-2-(methyl(3- | | |
| (trifluoromethyl)benzyl)amino)-6-oxo-1,6- | | |
| dihydropyrimidine-4-carboxylate | | |
| 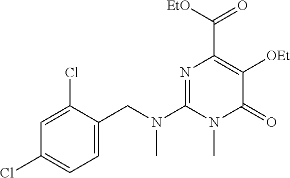 [SMILES: CCOC(=O)c1nc(N(C)Cc2ccc(Cl)cc2Cl)n(C)c(=O)c1OCC] | A16 | ESI-MS m/z = 414.5 [M + H]+. Calculated MW: 413.4 |
| ethyl 2-((2,4- | | |
| dichlorobenzyl)(methyl)amino)-5- | | |
| ethoxy-1-methyl-6-oxo-1,6- | | |
| dihydropyrimidine-4-carboxylate | | |
| 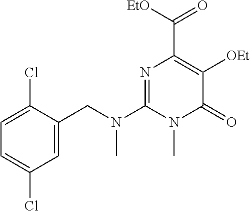 [SMILES: CCOC(=O)c1nc(N(C)Cc2cc(Cl)ccc2Cl)n(C)c(=O)c1OCC] | A17 | ESI-MS m/z = 414.5 [M + H]+. Calculated MW: 413.1 |
| ethyl 2-((2,5- | | |
| dichlorobenzyl)(methyl)amino)-5- | | |
| ethoxy-1-methyl-6-oxo-1,6- | | |
| dihydropyrimidine-4-carboxylate | | |
| 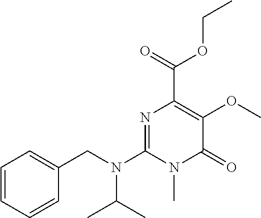 [SMILES: CCOC(=O)c1nc(N(Cc2ccccc2)C(C)C)n(C)c(=O)c1OC] | A28 | ESI-MS m/z = 360.2 [M + H]+. Calculated MW: 359.2 |
| ethyl 2- | | |
| (benzyl(isopropyl)amino)-5- | | |
| methoxy-1-methyl-6-oxo-1,6- | | |
| dihydropyrimidine-4- | | |
| carboxylate | | |
| 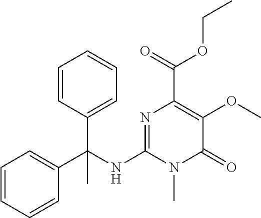 [SMILES: CCOC(=O)c1nc(NC(C)(c2ccccc2)c2ccccc2)n(C)c(=O)c1OC] | A37 | ESI-MS m/z = 408.4 [M + H]+. Calculated MW: 407.2 |
| ethyl 2-((1,1-diphenylethyl)amino)-5- | | |
| methoxy-1-methyl-6-oxo-1,6- | | |
| dihydropyrimidine-4-carboxylate | | |
| 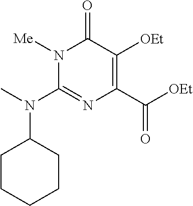 [SMILES: CCOC(=O)c1nc(N(C)C2CCCCC2)n(C)c(=O)c1OCC] | A47 | ESI-MS m/z = 338.4 [M + H]+. Calculated MW: 337.42 |
| ethyl 2-(cyclohexyl(methyl)amino)- | | |
| 5-ethoxy-1-methyl-6-oxo-1,6- | | |
| dihydropyrimidine-4-carboxylate | | |
| 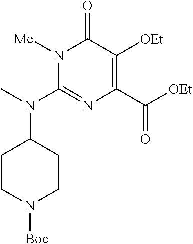 [SMILES: CCOC(=O)c1nc(N(C)C2CCN(C(=O)OC(C)(C)C)CC2)n(C)c(=O)c1OCC] | A48 | ESI-MS m/z = 439.4 [M + H]+. Calculated MW: 438.53 |
| ethyl 2-((1-(tert- | | |
| butoxycarbonyl)piperidin- | | |
| 4-yl)(methyl)amino)-5- | | |
| ethoxy-1-methyl-6-oxo- | | |
| 1,6-dihydropyrimidine-4- | | |
| carboxylate | | |
| 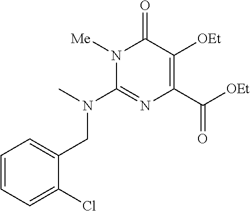 [SMILES: CCOC(=O)c1nc(N(C)Cc2ccccc2Cl)n(C)c(=O)c1OCC] | A49 | ESI-MS m/z = 380.5 [M + H]+. Calculated MW: 379.84 |
| ethyl 2-((2-chlorobenzyl)(methyl)amino)-5- | | |
| ethoxy-1-methyl-6-oxo-1,6- | | |
| dihydropyrimidine-4-carboxylate | | |
| 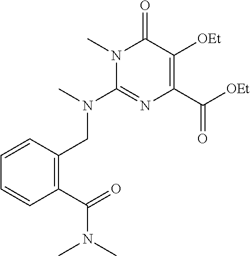 [SMILES: CCOC(=O)c1nc(N(C)Cc2ccccc2C(=O)N(C)C)n(C)c(=O)c1OCC] | A50 | ESI-MS m/z = 417.71 [M + H]+. Calculated MW: 416.48 |
| ethyl 2-((2- | | |
| (dimethylcarbamoyl)benzyl)(methyl) | | |
| amino)-5-ethoxy-1-methyl-6-oxo-1,6- | | |
| dihydropyrimidine-4-carboxylate | | |
| 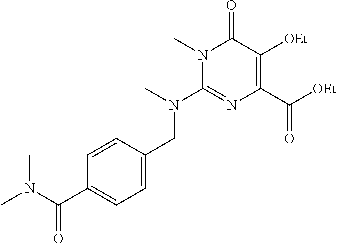 [SMILES: CCOC(=O)c1nc(N(C)Cc2ccc(C(=O)N(C)C)cc2)n(C)c(=O)c1OCC] | A51 | ESI-MS m/z = 417.8 [M + H]+. Calculated MW: 416.48 |
| ethyl 2-((4- | | |
| (dimethylcarbamoyl)benzyl)(methyl) | | |
| amino)-5-ethoxy-1-methyl-6-oxo- | | |
| 1,6-dihydropyrimidine-4-carboxylate | | |
| 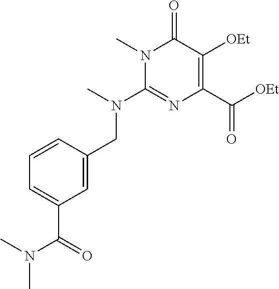 [SMILES: CCOC(=O)c1nc(N(C)Cc2cccc(C(=O)N(C)C)c2)n(C)c(=O)c1OCC] | A52 | ESI-MS m/z = 417.4 [M + H]+. Calculated MW: 416.48 |
| ethyl 2-((3- | | |
| (dimethylcarbamoyl)benzyl) | | |
| (methyl)amino)-5-ethoxy-1- | | |
| methyl-6-oxo-1,6-dihydropyrimidine- | | |
| 4-carboxylate | | |
| 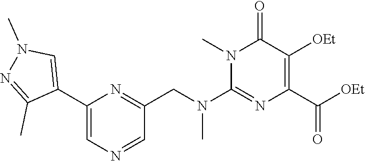 [SMILES: CCOC(=O)c1nc(N(C)Cc2cncc(-c3cn(C)nc3C)n2)n(C)c(=O)c1OCC] | A53 | ESI-MS m/z = 442.4 [M + H]+. Calculated MW: 441.49 |
| ethyl 2-(((6-(1,3-dimethyl-1H-pyrazol-4- | | |
| yl)pyrazin-2-yl)methyl)(methyl)amino)-5- | | |
| ethoxy-1-methyl-6-oxo-1,6- | | |
| dihydropyrimidine-4-carboxylate | | |
| 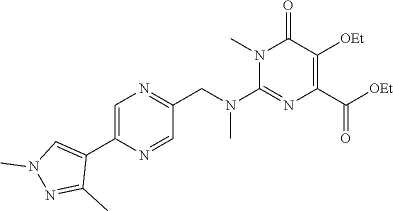 [SMILES: CCOC(=O)c1nc(N(C)Cc2cnc(-c3cn(C)nc3C)cn2)n(C)c(=O)c1OCC] | A54 | ESI-MS m/z = 442.40 [M + H]+. Calculated MW: 441.49 |
| ethyl 2-(((5-(1,3-dimethyl-1H-pyrazol-4- | | |
| yl)pyrazin-2-yl)methyl)(methyl)amino)-5- | | |
| ethoxy-1-methyl-6-oxo-1,6- | | |
| dihydropyrimidine-4-carboxylate | | |

### Synthesis of ethyl 2-(((2-chlorophenyl)(2-methylpyrimidin-5-yl)methyl)amino)-5-methoxy-1-methyl-6-oxo-1,6-dihydropyrimidine-4-carboxylate, A55

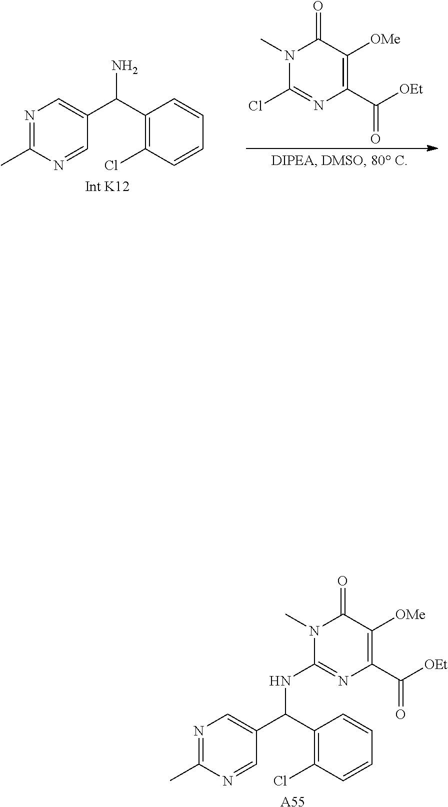

### Ethyl 2-(((2-chlorophenyl)(2-methylpyrimidin-5-yl)methyl)amino)-5-methoxy-1-methyl-6-oxo-1,6-dihydropyrimidine-4-carboxylate

To a stirred solution of (2-chlorophenyl)(2-methylpyrimidin-5-yl)methanamine (0.2 g, 0.85 mmol) and ethyl 2-chloro-5-methoxy-1-methyl-6-oxo-1,6-dihydropyrimidine-4-carboxylate (0.211 g, 0.85 mmol) in DMSO (2 mL) was added DIPEA (0.332 g, 2.57 mmol) and reaction mixture was heated at 100° C. for 3 h. Upon completion of reaction (monitored by TLC), the reaction mixture was diluted with ethyl acetate (40 mL), washed with cold water (3×30 mL) and brine (30 mL). The organic layer was separated, dried over sodium sulfate and evaporated to dryness to get crude compound. The obtained crude was purified by column chromatography in n-hexanes:ethyl acetate to obtain title compound (0.32 g, 84%). LCMS: ESI−MS m/z=444.30 [M+H]+. Calculated MW: 443.89

The following intermediates were synthesized using similar conditions as those described in the step above along with appropriate starting materials.

| Structure | Intermediate | LCMS |
|---|---|---|
| 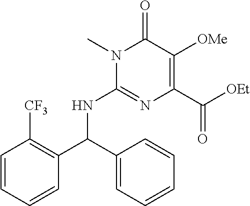 [SMILES: CCOC(=O)c1nc(NC(c2ccccc2)c2ccccc2C(F)(F)F)n(C)c(=O)c1OC] | A56 | ESI-MS m/z = 462.25 [M + H]+. Calculated MW: 461.44 |
| ethyl 5-methoxy-1-methyl-6-oxo-2- | | |
| ((phenyl(2- | | |
| (trifluoromethyl)phenyl)methyl)amino)- | | |
| 1,6-dihydropyrimidine-4-carboxylate | | |
| 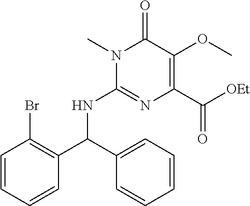 [SMILES: CCOC(=O)c1nc(NC(c2ccccc2)c2ccccc2Br)n(C)c(=O)c1OC] | A57 | ESI-MS m/z = 474.30 [M + 2H]+. Calculated MW: 472.34 |
| ethyl 2-(((2- | | |
| bromophenyl)(phenyl)methyl) | | |
| amino)-5-methoxy-1-methyl-6- | | |
| oxo-1,6-dihydropyrimidine-4- | | |
| carboxylate | | |
| 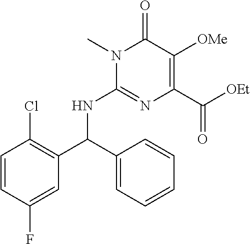 [SMILES: CCOC(=O)c1nc(NC(c2ccccc2)c2cc(F)ccc2Cl)n(C)c(=O)c1OC] | A58 | ESI-MS m/z = 446.35 [M + H]+. Calculated MW: 445.88 |
| ethyl 2-(((2-chloro-5- | | |
| fluorophenyl)(phenyl)methyl) | | |
| amino)-5-methoxy-1-methyl-6- | | |
| oxo-1,6-dihydropyrimidine-4- | | |
| carboxylate | | |
| 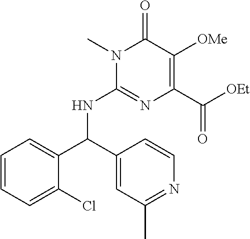 [SMILES: CCOC(=O)c1nc(NC(c2ccnc(C)c2)c2ccccc2Cl)n(C)c(=O)c1OC] | A59 | ESI-MS m/z = 443.40 [M + H]+. Calculated MW: 442.90 |
| ethyl 2-(((2-chlorophenyl)(2- | | |
| methylpyridin-4-yl)methyl)amino)- | | |
| 5-methoxy-1-methyl-6-oxo-1,6- | | |
| dihydropyrimidine-4-carboxylate | | |
| 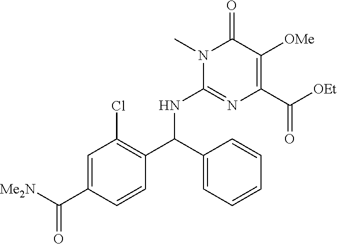 [SMILES: CCOC(=O)c1nc(NC(c2ccccc2)c2ccc(C(=O)N(C)C)cc2Cl)n(C)c(=O)c1OC] | A60 | ESI-MS m/z = 499.3 [M + H]+. Calculated MW: 498.96 |
| ethyl 2-(((2-chloro-4- | | |
| (dimethylcarbamoyl)phenyl) | | |
| (phenyl)methyl)amino)-5-methoxy- | | |
| 1-methyl-6-oxo-1,6-dihydro- | | |
| pyrimidine-4-carboxylate | | |
| 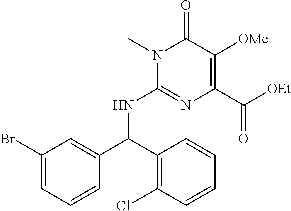 [SMILES: CCOC(=O)c1nc(NC(c2cccc(Br)c2)c2ccccc2Cl)n(C)c(=O)c1OC] | A61 | ESI-MS m/z = 508.7 [M + H]+. Calculated MW: 506.78 |
| ethyl 2-(((3-bromophenyl)(2- | | |
| chlorophenyl)methyl)amino)-5- | | |
| methoxy-1-methyl-6-oxo-1,6- | | |
| dihydropyrimidine-4-carboxylate | | |

### Synthesis of ethyl 2-(([1,1′-biphenyl]-2-ylmethyl)(methyl)amino)-5-methoxy-1-methyl-6-oxo-1,6-dihydropyrimidine-4-carboxylate, A18

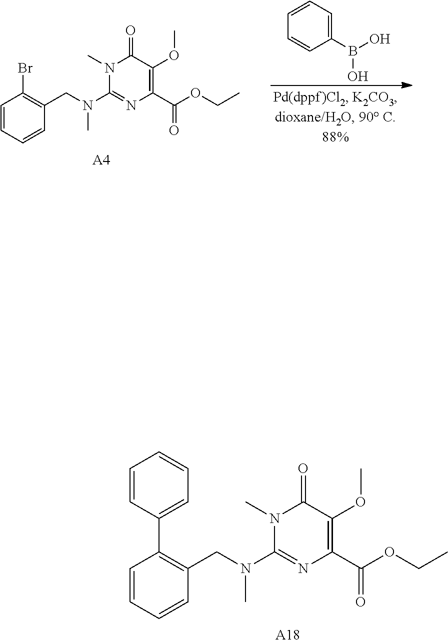

Into a 40-mL vial purged and maintained with an inert atmosphere of argon, was placed ethyl 2-[[(2-bromophenyl)methyl](methyl)amino]-5-methoxy-1-methyl-6-oxopyrimidine-4-carboxylate, A4 (500 mg, 1.22 mmol, 1.00 equiv), phenyl boronic acid (223 mg, 1.83 mmol, 1.50 equiv), Pd(dppf)Cl₂ (26.8 mg, 0.0370 mmol, 0.03 equiv), potassium carbonate (505 mg, 3.65 mmol, 3.00 equiv), 1,4-dioxane (7.00 mL), and water (1.40 mL). The resulting solution was stirred for 12 h at 95° C. in an oil bath. The solids were removed by filtration. The resulting mixture was concentrated. The residue was applied onto a silica gel column and eluted with ethyl acetate/petroleum ether (1:3). This resulted in 440 mg of (ethyl 2-([[1,1′-biphenyl]-2-ylmethyl](methyl)amino)-5-methoxy-1-methyl-6-oxopyrimidine-4-carboxylate) A18 (yield 88%) as yellow oil. Calculated MW: 407.2. ESI−MS m/z=408.3 [M+H]⁺

The following intermediates were synthesized using similar conditions as those described in the step above along with appropriate starting materials.

| Structure | Intermediate | LCMS |
|---|---|---|
| 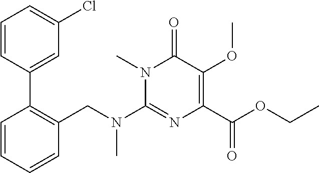 [SMILES: CCOC(=O)c1nc(N(C)Cc2ccccc2-c2cccc(Cl)c2)n(C)c(=O)c1OC] | A19 | ESI-MS m/z = 442.1 [M + H]+ Calculated MW: 441.1 |
| ethyl 2-(((3′-chloro-[1,1′-biphenyl]-2- | | |
| yl)methyl)(methyl)amino)-5-methoxy-1-methyl- | | |
| 6-oxo-1,6-dihydropyrimidine-4-carboxylate | | |
| 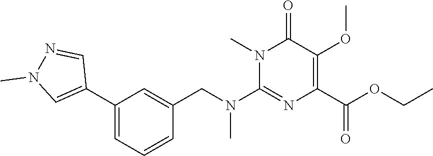 [SMILES: CCOC(=O)c1nc(N(C)Cc2cccc(-c3cnn(C)c3)c2)n(C)c(=O)c1OC] | A20 | ESI-MS m/z = 412.2 [M + H]+. Calculated MW: 411.2 |
| ethyl 5-methoxy-1-methyl-2-(methyl(3-(1- | | |
| methyl-1H-pyrazol-4-yl)benzyl)amino)-6- | | |
| oxo-1,6-dihydropyrimidine-4-carboxylate | | |
| 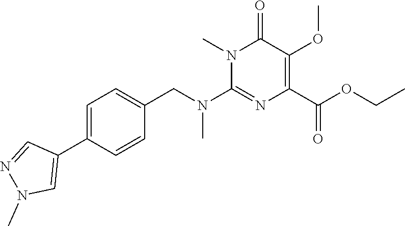 [SMILES: CCOC(=O)c1nc(N(C)Cc2ccc(-c3cnn(C)c3)cc2)n(C)c(=O)c1OC] | A25 | ESI-MS m/z = 412.2 [M + H]+. Calculated MW: 411.2 |
| ethyl 5-methoxy-1-methyl-2-(methyl(4-(1- | | |
| methyl-1H-pyrazol-4-yl)benzyl)amino)-6- | | |
| oxo-1,6-dihydropyrimidine-4-carboxylate | | |
| 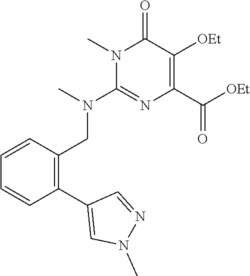 [SMILES: CCOC(=O)c1nc(N(C)Cc2ccccc2-c2cnn(C)c2)n(C)c(=O)c1OCC] | A53 | ESI-MS m/z = 426.6 [M + H]+. Calculated MW: 425.2 |
| ethyl 5-ethoxy-1-methyl-2- | | |
| (methyl(2-(1-methyl-1H-pyrazol- | | |
| 4-yl)benzyl)amino)-6-oxo-1,6- | | |
| dihydropyrimidine-4-carboxylate | | |

### Synthesis of ethyl 2-[(diphenylmethyl) (methyl) amino]-5-methoxy-1-methyl-6-oxopyrimidine-4-carboxylate, A29-2

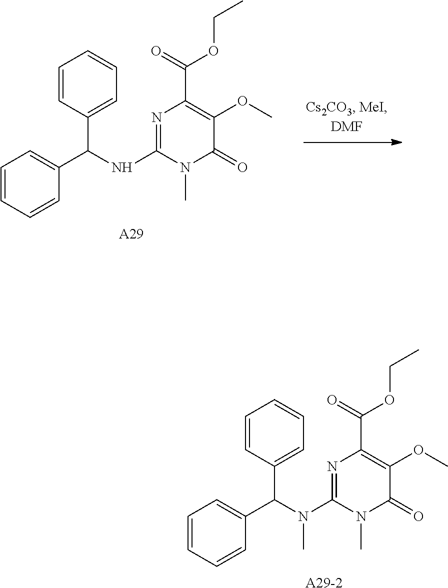

To a stirred mixture of ethyl 2-[(diphenylmethyl)amino]-5-methoxy-1-methyl-6-oxopyrimidine-4-carboxylate (4.2 g, 10.7 mmol, 1.00 equiv) and cesium carbonate (7.0 g, 21.4 mmol, 2 equiv) in N,N-dimethyl formamide (100 mL) was added methyl iodide (4.6 g, 32.1 mmol, 3.00 equiv) in portions for 4 h at room temperature. The resulting mixture was extracted with ethyl acetate. The combined organic layers were washed with brine, dried over anhydrous sodium sulfate. After filtration, the filtrate was concentrated under reduced pressure. The residue was applied onto a reversed-phase column with Acetonitrile/Water (0.1% FA) (4:1). This resulted in 3.1 g of ethyl 2-[(diphenylmethyl) (methyl) amino]-5-methoxy-1-methyl-6-oxopyrimidine-4-carboxylate, A29-2 (yield 66%) as a yellow solid. ESI−MS m/Z=408.3 [M+H]⁺ Calculated MW: 407.1

The following intermediates were synthesized using similar conditions as those described in the step above along with appropriate starting materials.

| Structure | Intermediate | LCMS |
|---|---|---|
| 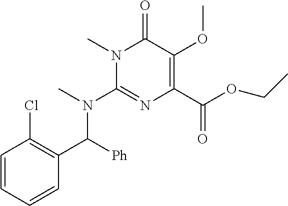 [SMILES: CCOC(=O)c1nc(N(C)C(c2ccccc2)c2ccccc2Cl)n(C)c(=O)c1OC] | A30-2 | ESI-MS m/z = 442.1 [M + H]+. Calculated MW: 441.2 |
| 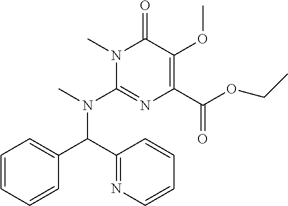 [SMILES: CCOC(=O)c1nc(N(C)C(c2ccccc2)c2ccccn2)n(C)c(=O)c1OC] | A31-2 | ESI-MS m/z = 409.4 [M + H]+ Calculated MW: 408.2 |
| ethyl 5-methoxy-1-methyl- | | |
| 2-(methyl(phenyl(pyridin-2- | | |
| yl)methyl)amino)-6-oxo- | | |
| 1,6-dihydropyrimidine-4- | | |
| carboxylate | | |
| 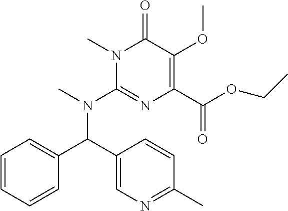 [SMILES: CCOC(=O)c1nc(N(C)C(c2ccccc2)c2ccc(C)nc2)n(C)c(=O)c1OC] | A32-2 | ESI-MS m/z = 423.2 [M + H]+ Calculated MW: 422.2 |
| ethyl 5-methoxy-1- | | |
| methyl-2- | | |
| (methyl((6- | | |
| methylpyridin-3- | | |
| yl)(phenyl)methyl) | | |
| amino)-6-oxo-1,6- | | |
| dihydropyrimidine- | | |
| 4-carboxylate | | |
| 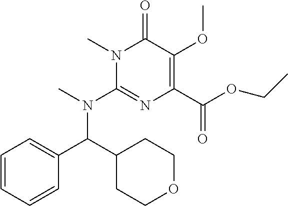 [SMILES: CCOC(=O)c1nc(N(C)C(c2ccccc2)C2CCOCC2)n(C)c(=O)c1OC] | A33-2 | ESI-MS m/z = 416.4 [M + H]+. Calculated MW: 415.2 |
| ethyl 5-methoxy-1-methyl-2- | | |
| (methyl(phenyl(tetrahydro- | | |
| 2H-pyran-4-yl)methyl)amino)- | | |
| 6-oxo-1,6-dihydropyrimidine- | | |
| 4-carboxylate | | |
| 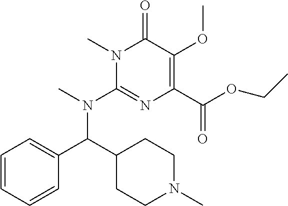 [SMILES: CCOC(=O)c1nc(N(C)C(c2ccccc2)C2CCN(C)CC2)n(C)c(=O)c1OC] | A35-2 | ESI-MS m/z = 429.5 [M + H]+. Calculated MW: 428.2 |
| ethyl 5-methoxy-1-methyl-2- | | |
| (methyl((1-methylpiperidin-4- | | |
| yl)(phenyl)methyl)amino)-6-oxo- | | |
| 1,6-dihydropyrimidine-4- | | |
| carboxylate | | |
| 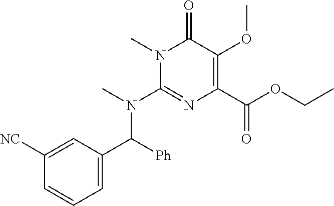 [SMILES: CCOC(=O)c1nc(N(C)C(c2ccccc2)c2cccc(C#N)c2)n(C)c(=O)c1OC] | A36-2 | ESI-MS m/z = 433.1 [M + H]+. Calculated MW: 432.2 |
| ethyl 2-(((3- | | |
| cyanophenyl)(phenyl)methyl)(methyl)amino)-5- | | |
| methoxy-1-methyl-6-oxo-1,6-dihydropyrimidine- | | |
| 4-carboxylate | | |
| 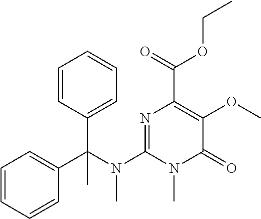 [SMILES: CCOC(=O)c1nc(N(C)C(C)(c2ccccc2)c2ccccc2)n(C)c(=O)c1OC] | A37-2 | ESI-MS m/z = 422.5 [M + H]+. Calculated MW: 421.2 |
| ethyl 2-((1,1- | | |
| diphenylethyl)(methyl)amino)-5- | | |
| methoxy-1-methyl-6-oxo-1,6- | | |
| dihydropyrimidine-4-carboxylate | | |
| 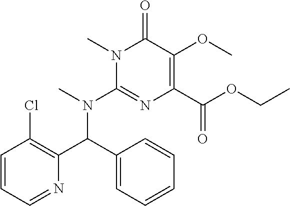 [SMILES: CCOC(=O)c1nc(N(C)C(c2ccccc2)c2ncccc2Cl)n(C)c(=O)c1OC] | A38-2 | ESI-MS m/z = 443.2 [M + H]+. Calculated MW: 442.1 |
| ethyl 2-(((3-chloropyridin-2- | | |
| yl)(phenyl)methyl)(methyl)amino)- | | |
| 5-methoxy-1-methyl-6-oxo-1,6- | | |
| dihydropyrimidine-4-carboxylate | | |
| 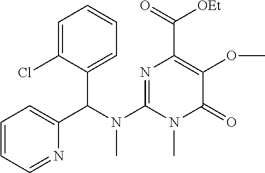 [SMILES: CCOC(=O)c1nc(N(C)C(c2ccccn2)c2ccccc2Cl)n(C)c(=O)c1OC] | A40-2 | ESI-MS m/z = 443.4 [M + H]+. Calculated MW: 442.1 |
| ethyl 2-(((2-chlorophenyl)(pyridin-2- | | |
| yl)methyl)(methyl)amino)-5-methoxy-1- | | |
| methyl-6-oxo-1,6-dihydropyrimidine-4- | | |
| carboxylate | | |
| 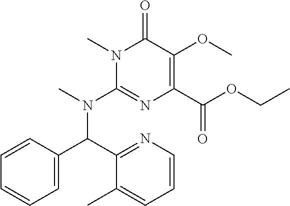 [SMILES: CCOC(=O)c1nc(N(C)C(c2ccccc2)c2ncccc2C)n(C)c(=O)c1OC] | A41-2 | ESI-MS m/z = 423.4 [M + H]+. Calculated MW: 422.2 |
| ethyl 5-methoxy-1-methyl-2-(methyl((3- | | |
| methylpyridin-2-yl)(phenyl)methyl)amino)- | | |
| 6-oxo-1,6-dihydropyrimidine-4-carboxylate | | |
| 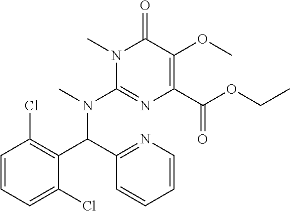 [SMILES: CCOC(=O)c1nc(N(C)C(c2ccccn2)c2c(Cl)cccc2Cl)n(C)c(=O)c1OC] | A42-2 | ESI-MS m/z = 477.0 [M + H]+. Calculated MW: 476.1 |
| ethyl 2-(((2,6-dichlorophenyl)(pyridin- | | |
| 2-yl)methyl)(methyl)amino)-5- | | |
| methoxy-1-methyl-6-oxo-1,6- | | |
| dihydropyrimidine-4-carboxylate | | |
| 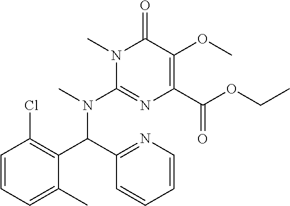 [SMILES: CCOC(=O)c1nc(N(C)C(c2ccccn2)c2c(C)cccc2Cl)n(C)c(=O)c1OC] | A43-2 | ESI-MS m/z = 457.1 [M + H]+. Calculated MW: 456.2 |
| ethyl 2-(((2-chloro-6- | | |
| methylphenyl)(pyridin-2- | | |
| yl)methyl)(methyl)amino)-5- | | |
| methoxy-1-methyl-6-oxo-1,6- | | |
| dihydropyrimidine-4-carboxylate | | |
| 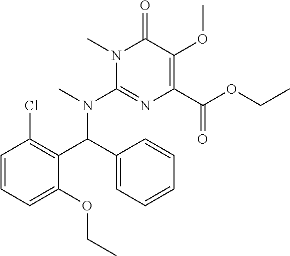 [SMILES: CCOC(=O)c1nc(N(C)C(c2ccccc2)c2c(Cl)cccc2OCC)n(C)c(=O)c1OC] | A44-2 | ESI-MS m/z = 486.2 [M + H]+. Calculated MW: 485.2 |
| ethyl 2-(((2-chloro-6- | | |
| ethoxyphenyl)(phenyl)methyl) | | |
| (methyl)amino)-5-methoxy-1-methyl- | | |
| 6-oxo-1,6-dihydropyrimidine-4- | | |
| carboxylate | | |
| 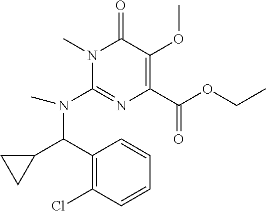 [SMILES: CCOC(=O)c1nc(N(C)C(c2ccccc2Cl)C2CC2)n(C)c(=O)c1OC] | A45-2 | ESI-MS m/z = 406.0 [M + H]+. Calculated MW: 405.2 |
| ethyl 2-(((2- | | |
| chlorophenyl)(cyclopropyl)methyl) | | |
| (methyl)amino)-5-methoxy-1-methyl-6- | | |
| oxo-1,6-dihydropyrimidine-4-carboxylate | | |
| 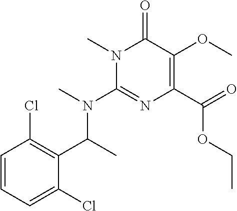 [SMILES: CCOC(=O)c1nc(N(C)C(C)c2c(Cl)cccc2Cl)n(C)c(=O)c1OC] | A46-2 | ESI-MS m/z = 414.2 [M + H]+. Calculated MW: 413.1 |
| ethyl 2-((1-(2,6- | | |
| dichlorophenyl)ethyl)(methyl) | | |
| amino)-5-methoxy-1-methyl-6- | | |
| oxo-1,6-dihydropyrimidine-4- | | |
| carboxylate | | |
| 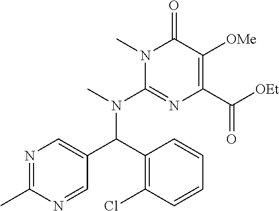 [SMILES: CCOC(=O)c1nc(N(C)C(c2cnc(C)nc2)c2ccccc2Cl)n(C)c(=O)c1OC] | A55-2 | ESI-MS m/z = 458.41 [M + H]+. Calculated MW: 457.92 |
| ethyl 2-(((2-chlorophenyl)(2- | | |
| methylpyrimidin-5- | | |
| yl)methyl)(methyl)amino)-5- | | |
| methoxy-1-methyl-6-oxo-1,6- | | |
| dihydropyrimidine-4-carboxylate | | |
| 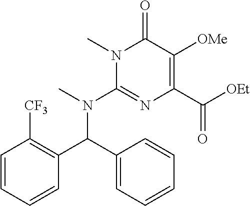 [SMILES: CCOC(=O)c1nc(N(C)C(c2ccccc2)c2ccccc2C(F)(F)F)n(C)c(=O)c1OC] | A56-2 | ESI-MS m/z = 476.4 [M + H]+. Calculated MW: 475.47 |
| ethyl 5-methoxy-1-methyl-2- | | |
| (methyl(phenyl(2- | | |
| (trifluoromethyl)phenyl)methyl) | | |
| amino)-6-oxo-1,6- | | |
| dihydropyrimidine-4-carboxylate | | |
| 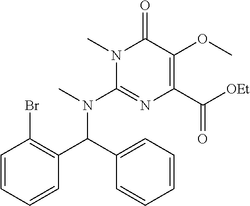 [SMILES: CCOC(=O)c1nc(N(C)C(c2ccccc2)c2ccccc2Br)n(C)c(=O)c1OC] | A57-2 | ESI-MS m/z = 488.20 [M + 2H]+. Calculated MW: 486.37 |
| ethyl 2-(((2- | | |
| bromophenyl)(phenyl)methyl) | | |
| (methyl)amino)-5-methoxy-1- | | |
| methyl-6-oxo-1,6- | | |
| dihydropyrimidine-4- | | |
| carboxylate | | |
| 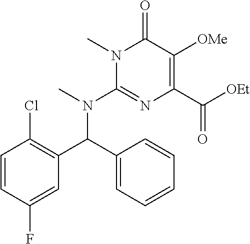 [SMILES: CCOC(=O)c1nc(N(C)C(c2ccccc2)c2cc(F)ccc2Cl)n(C)c(=O)c1OC] | A58-2 | ESI-MS m/z = 460.61 [M + H]+. Calculated MW: 459.9 |
| ethyl 2-(((2-chloro-5- | | |
| fluorophenyl)(phenyl)methyl) | | |
| (methyl)amino)-5-methoxy- | | |
| 1-methyl-6-oxo-1,6- | | |
| dihydropyrimidine-4- | | |
| carboxylate | | |
| 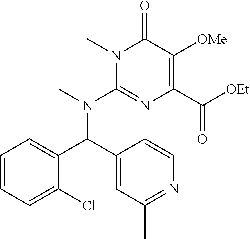 [SMILES: CCOC(=O)c1nc(N(C)C(c2ccnc(C)c2)c2ccccc2Cl)n(C)c(=O)c1OC] | A59-2 | ESI-MS m/z = 457.40 [M + H]+. Calculated MW: 456.93 |
| ethyl 2-(((2-chlorophenyl)(2- | | |
| methylpyridin-4- | | |
| yl)methyl)(methyl)amino)-5- | | |
| methoxy-1-methyl-6-oxo-1,6- | | |
| dihydropyrimidine-4-carboxylate | | |
| 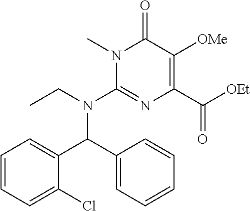 [SMILES: CCOC(=O)c1nc(N(CC)C(c2ccccc2)c2ccccc2Cl)n(C)c(=O)c1OC] | A40-3 | ESI-MS m/z = 456.24 [M + H]+. Calculated MW: 455.94 |
| ethyl 2-(((2- | | |
| chlorophenyl)(phenyl)methyl) | | |
| (ethyl)amino)-5-methoxy-1- | | |
| methyl-6-oxo-1,6- | | |
| dihydropyrimidine-4- | | |
| carboxylate | | |
| 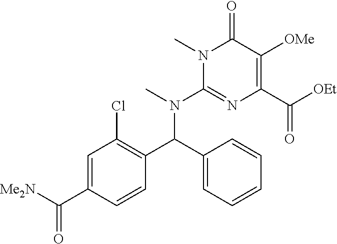 [SMILES: CCOC(=O)c1nc(N(C)C(c2ccccc2)c2ccc(C(=O)N(C)C)cc2Cl)n(C)c(=O)c1OC] | A60-2 | ESI-MS m/z = 513.8 [M + H]+. Calculated MW: 512.99 |
| ethyl 2-(((2-chloro-4- | | |
| (dimethylcarbamoyl)phenyl)(phenyl) | | |
| methyl)(methyl)amino)-5-methoxy-1- | | |
| methyl-6-oxo-1,6-dihydropyrimidine- | | |
| 4-carboxylate | | |
| 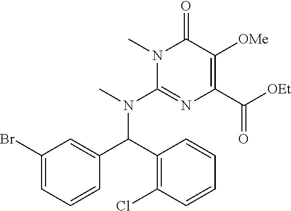 [SMILES: CCOC(=O)c1nc(N(C)C(c2cccc(Br)c2)c2ccccc2Cl)n(C)c(=O)c1OC] | A61-2 | ESI-MS m/z = 522.7 [M + 2H]+. Calculated MW: 520.81 |
| ethyl 2-(((3-bromophenyl)(2- | | |
| chlorophenyl)methyl)(methyl) | | |
| amino)-5-methoxy-1-methyl-6- | | |
| oxo-1,6-dihydropyrimidine-4- | | |
| carboxylate | | |
| 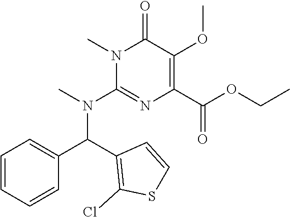 [SMILES: CCOC(=O)c1nc(N(C)C(c2ccccc2)c2ccsc2Cl)n(C)c(=O)c1OC] | A62-2 | ESI-MS m/z = 447.9 [M + H]+. Calculated MW: 447.1 |
| ethyl 2-(((2-chlorothiophen-3- | | |
| yl)(phenyl)methyl)(methyl)amino)- | | |
| 5-methoxy-1-methyl-6-oxo-1,6- | | |
| dihydropyrimidine-4-carboxylate | | |

### Chiral separation of O-methylhydroxypyrimidinone esters

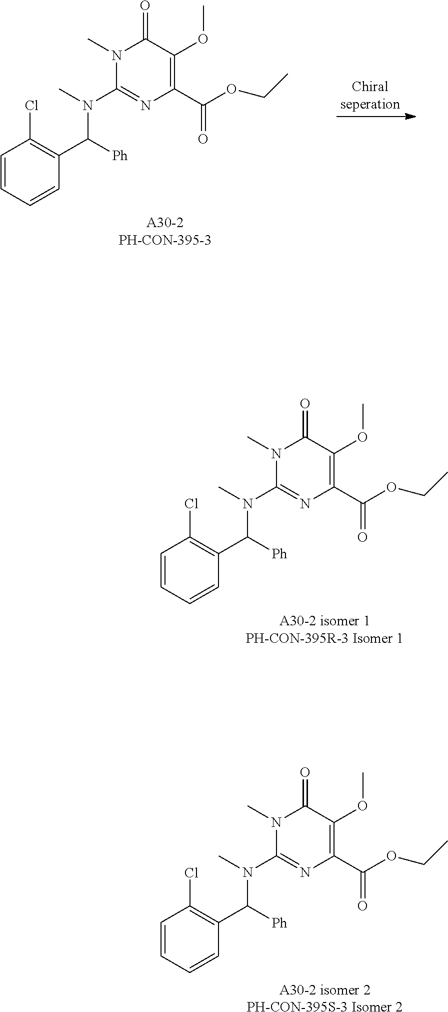 [SMILES: CCOC(=O)c1nc(N(C)C(c2ccccc2)c2ccccc2Cl)n(C)c(=O)c1OC ; "A30-2PH-CON-395-3" ; CCOC(=O)c1nc(N(C)C(c2ccccc2)c2ccccc2Cl)n(C)c(=O)c1OC ; "A30-2 isomer 1PH-CON-395R-3 Isomer 1" ; CCOC(=O)c1nc(N(C)C(c2ccccc2)c2ccccc2Cl)n(C)c(=O)c1OC ; "A30-2 isomer 2PH-CON-395S-3 Isomer 2"]

The racemate of PH-CON-395-3 (ethyl 2-[[(2-chlorophenyl)(phenyl)methyl](methyl)amino]-5-methoxy-1-methyl-6-oxopyrimidine-4-carboxylate) 800 mg was purified by prep-chiral-HPLC with the following conditions (Column: CHIRALPAK IC SFC (02), 5*25 cm, 5 um; Mobile Phase A:CO₂, Mobile Phase B:IPA (2 mM NH₃-MeOH); Flow rate: 1.80 mL/min; Gradient: 50% B; 220 nm; retention time of isomer 1: 6.15 min retention time of isomer 2: 7.55 min; injection volume: 5 mL; number of runs:16)

Isolation of the left peak, at 6.15 min, resulted in 360 mg of A30-2 isomer 1 ((ethyl 2-[[(2-chlorophenyl)(phenyl)methyl](methyl)amino]-5-methoxy-1-methyl-6-oxopyrimidine-4-carboxylate) as a white solid.

Isolation of the right peak, at 7.55 min, resulted in 360 mg of A30-2 isomer 2 (ethyl 2-[[(2-chlorophenyl)(phenyl)methyl](methyl)amino]-5-methoxy-1-methyl-6-oxopyrimidine-4-carboxylate) as a white solid.

The following intermediates were separated using similar conditions as those described above.

| | | Chiral Separation | Retention |
|---|---|---|---|
| Structure | Intermediate | Conditions | Time |
| 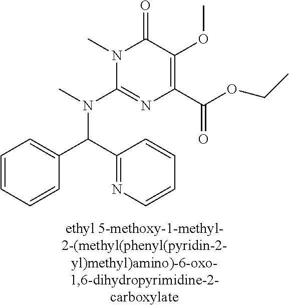 [SMILES: CCOC(=O)c1nc(N(C)C(c2ccccc2)c2ccccn2)n(C)c(=O)c1OC] | A31-2 Isomer 1 | CHIRALPAK AD-3 column (0.46*5 cm, 3 um) hexanes (0.1% diethylamine)/ ethanol 70:30 ratio flow rate = 1.0 mL/min | 2.19 min (Distomer) |
| 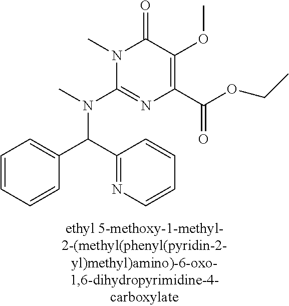 [SMILES: CCOC(=O)c1nc(N(C)C(c2ccccc2)c2ccccn2)n(C)c(=O)c1OC] | A31-2 Isomer 2 | CHIRALPAK AD-3 column (0.46*5 cm, 3 um) hexanes (0.1% diethylamine)/ ethanol 70:30 ratio flow rate = 1.0 mL/min | 3.44 min (Eutomer) |
| 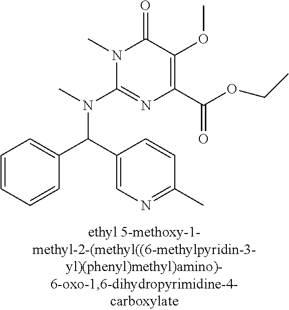 [SMILES: CCOC(=O)c1nc(N(C)C(c2ccccc2)c2ccc(C)nc2)n(C)c(=O)c1OC] | A32-2 Isomer 1 | CHIRALPAK IA, 0.46*5 cm, 3 um Mobile Phase: Hex (0.1% DEA):IP A = 70:30 Flow rate: 1 mL/min; | 2.80 min (Distomer) |
| 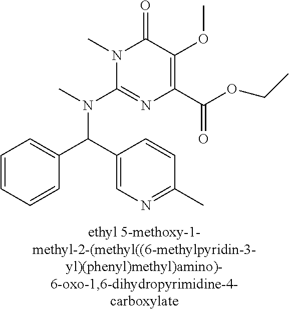 [SMILES: CCOC(=O)c1nc(N(C)C(c2ccccc2)c2ccc(C)nc2)n(C)c(=O)c1OC] | A32-2 Isomer 2 | CHIRALPAK IA, 0.46*5 cm, 3 um Mobile Phase: Hex (0.1% DEA):IP A = 70:30 Flow rate: 1 mL/min; | 3.70 min (Eutomer) |
| 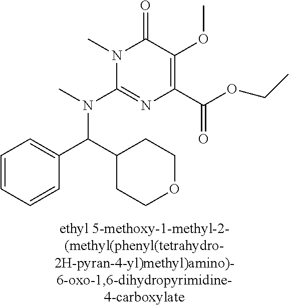 [SMILES: CCOC(=O)c1nc(N(C)C(c2ccccc2)C2CCOCC2)n(C)c(=O)c1OC] | A33-2 Isomer 1 | CHIRALPAK AD-3 column (4.6*100 mm, 3 um) hexanes (0.1% diethylamine)/ ethanol 50:50 ratio flow rate = 1.0 mL/min | 3.20 (Distomer) |
| 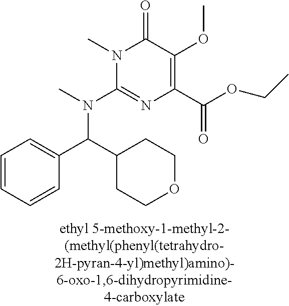 [SMILES: CCOC(=O)c1nc(N(C)C(c2ccccc2)C2CCOCC2)n(C)c(=O)c1OC] | A33-2 Isomer 2 | CHIRALPAK AD-3 column (4.6*100 mm, 3 um) hexanes (0.1% diethylamine)/ ethanol 50:50 ratio flow rate = 1.0 mL/min | 5.00 min (Eutomer) |
| 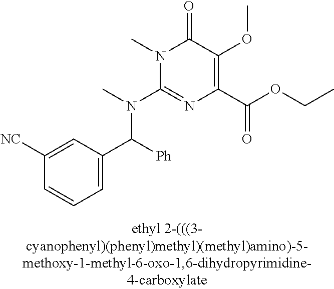 [SMILES: CCOC(=O)c1nc(N(C)C(c2ccccc2)c2cccc(C#N)c2)n(C)c(=O)c1OC] | A36-2 Isomer 1 | CHIRAL ART Cellulose-SB, 4.6*100 mm, ,3 μm; Mobile Phase A: Hex (0.1% DEA): ethanol = 70:30, Mobile Phase B; Flow rate: 1 mL/min | 4.05 min (Eutomer) |
| 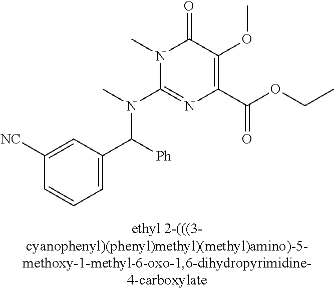 [SMILES: CCOC(=O)c1nc(N(C)C(c2ccccc2)c2cccc(C#N)c2)n(C)c(=O)c1OC] | A36-2 Isomer 2 | CHIRAL ART Cellulose-SB, 4.6*100 mm, 3 μm; Mobile Phase A: Hex (0.1% DEA): ethanol = 70:30, Mobile Phase B; Flow rate: 1 mL/min | 4.70 min (Distomer) |
| 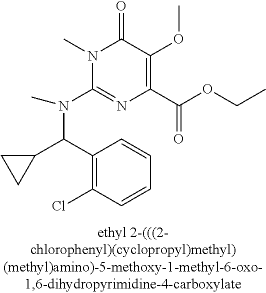 [SMILES: CCOC(=O)c1nc(N(C)C(c2ccccc2Cl)C2CC2)n(C)c(=O)c1OC] | A45 Isomer 1 | CHIRALPAK IG-3 column (0.46*5 cm, 3 um) Mobile Phase A: Hex (0.1% DEA): EtOH = 70:30; flow rate = 1.0 mL/min | 2.81 min (Distomer) |
| 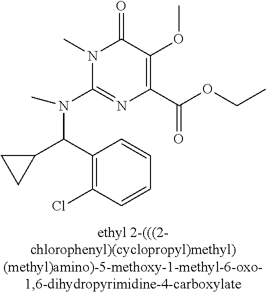 [SMILES: CCOC(=O)c1nc(N(C)C(c2ccccc2Cl)C2CC2)n(C)c(=O)c1OC] | A45 Isomer 2 | CHIRALPAK IG-3 column (0.46*5 cm, 3 um) Mobile Phase A: Hex (0.1% DEA): EtOH = 70:30; flow rate = 1.0 mL/min | 4.56 (Distomer) |

### Synthesis of ethyl 2-(((2-cyanophenyl)(phenyl)methyl)(methyl)amino)-5-methoxy-1-methyl-6-oxo-1,6-dihydropyrimidine-4-carboxylate, A57-3

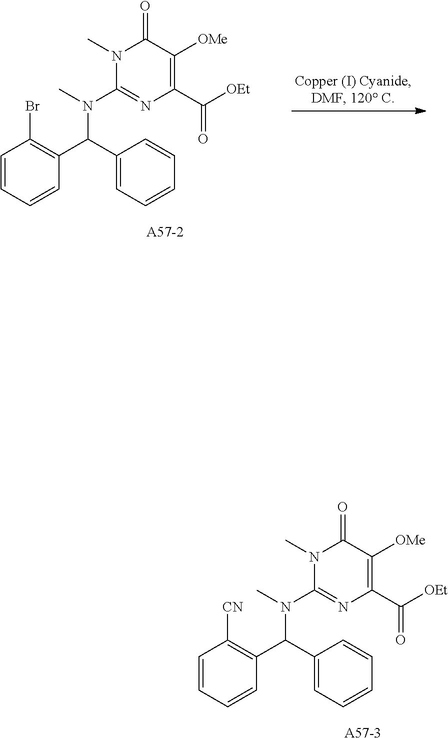

### Ethyl 2-(((2-cyanophenyl)(phenyl)methyl)(methyl)amino)-5-methoxy-1-methyl-6-oxo-1,6-dihydropyrimidine-4-carboxylate

Ethyl 2-(((2-bromophenyl)(phenyl)methyl)(methyl) amino)-5-methoxy-1-methyl-6-oxo-1,6-dihydropyrimidine-4-carboxylate (0.47 g, 0.96 mmol) was dissolved in DMF (4.7 mL) and copper(I) cyanide (0.26 g, 2.9 mmol) was added to the solution. The reaction mixture was heated to 150° C. for 16 h. Upon completion of reaction (monitored by TLC), the reaction mixture was quenched with water, extracted in ethyl acetate (3×30 mL), dried over sodium sulphate and concentrated under reduced pressure to get crude compound. The obtained crude compound was purified by column chromatography in n-hexanes:ethyl acetate to obtain pure title compound (0.30 g, 71%). LCMS: ESI−MS m/z=433.19 [M+H]+. Calculated MW: 432.48

The following intermediates were synthesized using similar conditions as those described in the step above along with appropriate starting materials.

| | Inter- | |
|---|---|---|
| Structure | mediate | LCMS |
| 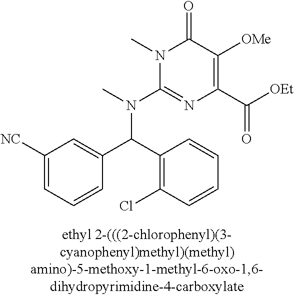 [SMILES: CCOC(=O)c1nc(N(C)C(c2cccc(C#N)c2)c2ccccc2Cl)n(C)c(=O)c1OC] | A61-3 | ESI-MS m/z = 467.2 [M + H]+ Calculated MW: 466.1 |

### Synthesis of 2-(dibenzylamino)-5-ethoxy-1-methyl-6-oxo-1,6-dihydropyrimidine-4-carboxylic acid, B1

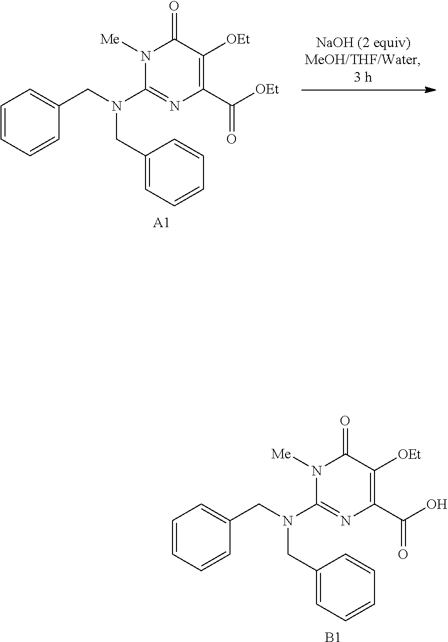

To a stirred solution of ethyl 2-(dibenzylamino)-5-ethoxy-1-methyl-6-oxo-1,6-dihydropyrimidine-4-carboxylate (130 mg, 308 μmol) and lithium hydroxide (14.7 mg, 616 μmol) in THF (1.5 mL) and water (0.75 mL) was stirred at room temperature for 3 h. The resulting mixture was concentrated under vacuum, and the crude B1 was directly used in the next step. Calculated MW: 393.4 ESI−MS m/z=394.2 [M+H]⁺.

The following intermediates were synthesized using similar conditions as those described in the step above along with appropriate starting materials.

| Structure | Intermediate | LCMS |
|---|---|---|
| 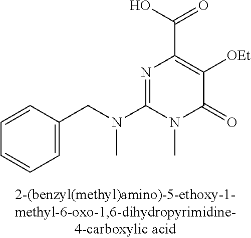 [SMILES: CCOc1c(C(=O)O)nc(N(C)Cc2ccccc2)n(C)c1=O] | B2 | ESI-MS m/z = 318.3 [M + H]+. Calculated MW: 317.3 |
| 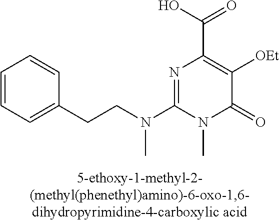 [SMILES: CCOc1c(C(=O)O)nc(N(C)CCc2ccccc2)n(C)c1=O] | B3 | ESI-MS m/z = 332.5 [M + H]+. Calculated MW: 331.3 |
| 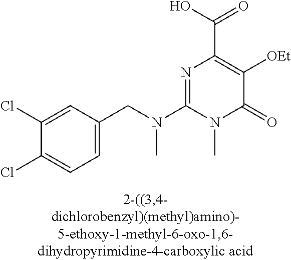 [SMILES: CCOc1c(C(=O)O)nc(N(C)Cc2ccc(Cl)c(Cl)c2)n(C)c1=O] | B4 | ESI-MS m/z = 386.2 [M + H]+. Calculated MW: 385.1 |
| 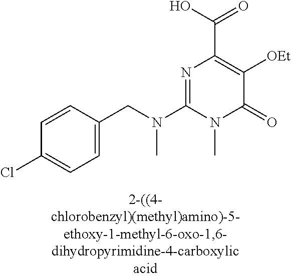 [SMILES: CCOc1c(C(=O)O)nc(N(C)Cc2ccc(Cl)cc2)n(C)c1=O] | B5 | ESI-MS m/z = 352.2 [M + H]+. Calculated MW: 351.8 |
| 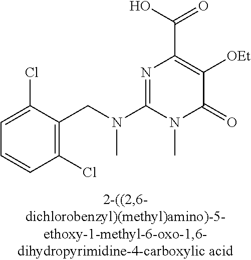 [SMILES: CCOc1c(C(=O)O)nc(N(C)Cc2c(Cl)cccc2Cl)n(C)c1=O] | B6 | ESI-MS m/z = 386.2 [M + H]+. Calculated MW: 385..1 |
| 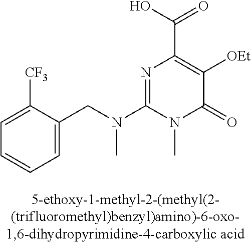 [SMILES: CCOc1c(C(=O)O)nc(N(C)Cc2ccccc2C(F)(F)F)n(C)c1=O] | B7 | ESI-MS m/z = 386.25 [M + H]+. Calculated MW: 385.3 |
| 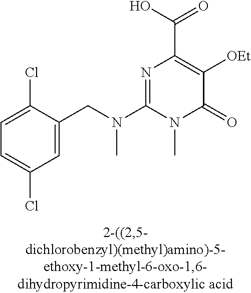 [SMILES: CCOc1c(C(=O)O)nc(N(C)Cc2cc(Cl)ccc2Cl)n(C)c1=O] | B8 | ESI-MS m/z = 386.2 [M + H]+. Calculated MW: 385..1 |
| 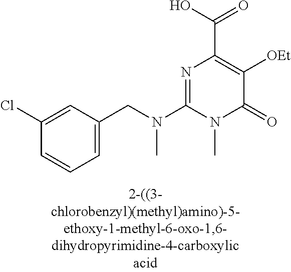 [SMILES: CCOc1c(C(=O)O)nc(N(C)Cc2cccc(Cl)c2)n(C)c1=O] | B9 | ESI-MS m/z = 352.2 [M + H]+. Calculated MW: 351.8 |
| 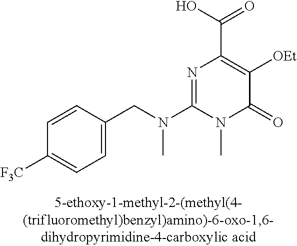 [SMILES: CCOc1c(C(=O)O)nc(N(C)Cc2ccc(C(F)(F)F)cc2)n(C)c1=O] | B10 | ESI-MS m/z = 386.3 [M + H]+. Calculated MW: 385.3 |
| 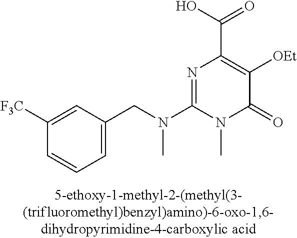 [SMILES: CCOc1c(C(=O)O)nc(N(C)Cc2cccc(C(F)(F)F)c2)n(C)c1=O] | B11 | ESI-MS m/z = 386.3 [M + H]+. Calculated MW: 385.3 |
| 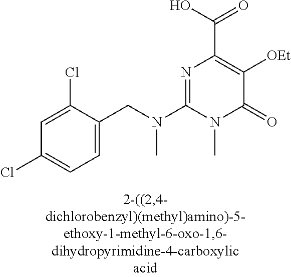 [SMILES: CCOc1c(C(=O)O)nc(N(C)Cc2ccc(Cl)cc2Cl)n(C)c1=O] | B12 | ESI-MS m/z = 386.2 [M + H]+. Calculated MW: 385..1 |
| 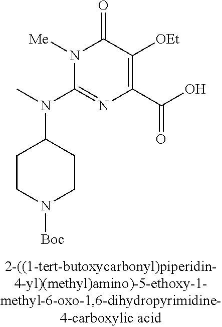 [SMILES: CCOc1c(C(=O)O)nc(N(C)C2CCN(C(=O)OC(C)(C)C)CC2)n(C)c1=O] | B52 | ESI-MS m/z = 411.2 [M + H]+. Calculated MW: 410.47 |
| 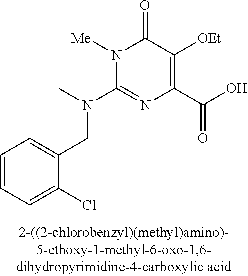 [SMILES: CCOc1c(C(=O)O)nc(N(C)Cc2ccccc2Cl)n(C)c1=O] | B53 | ESI-MS m/z = 352.2 [M + H]+. Calculated MW: 351.79 |
| 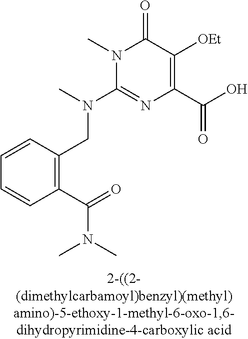 [SMILES: CCOc1c(C(=O)O)nc(N(C)Cc2ccccc2C(=O)N(C)C)n(C)c1=O] | B54 | ESI-MS m/z = 389.30 [M + H]+. Calculated MW: 388.42 |
| 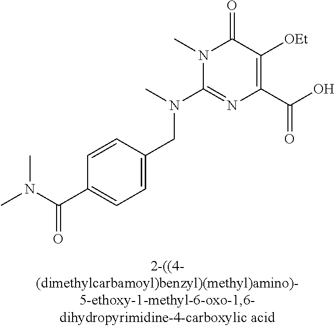 [SMILES: CCOc1c(C(=O)O)nc(N(C)Cc2ccc(C(=O)N(C)C)cc2)n(C)c1=O] | B55 | ESI-MS m/z = 389.30 [M + H]+. Calculated MW: 388.42 |
| 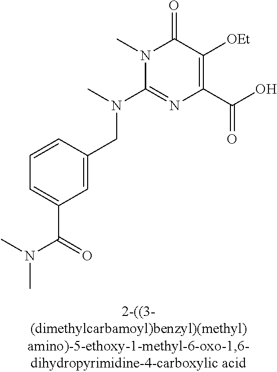 [SMILES: CCOc1c(C(=O)O)nc(N(C)Cc2cccc(C(=O)N(C)C)c2)n(C)c1=O] | B56 | ESI-MS m/z = 389.54 [M + H]+. Calculated MW: 388.42 |
| 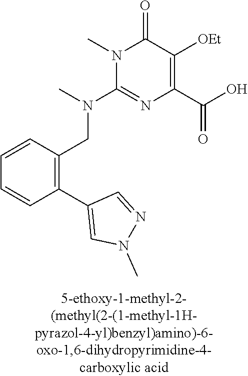 [SMILES: CCOc1c(C(=O)O)nc(N(C)Cc2ccccc2-c2cnn(C)c2)n(C)c1=O] | B57 | ESI-MS m/z = 398.2 [M + H]+. Calculated MW: 397.44 |
| 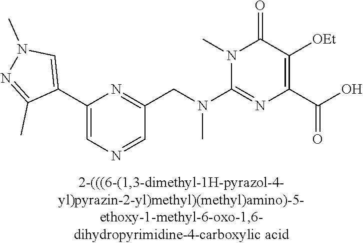 [SMILES: CCOc1c(C(=O)O)nc(N(C)Cc2cncc(-c3cn(C)nc3C)n2)n(C)c1=O] | B58 | ESI-MS m/z = 414.36 [M + H]+. Calculated MW: 413.44 |
| 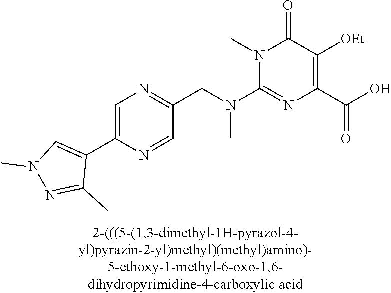 [SMILES: CCOc1c(C(=O)O)nc(N(C)Cc2cnc(-c3cn(C)nc3C)cn2)n(C)c1=O] | B59 | ESI-MS m/z = 414.4 [M + H]+. Calculated MW: 413.44 |
| 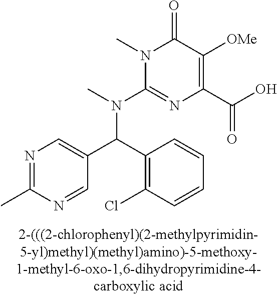 [SMILES: COc1c(C(=O)O)nc(N(C)C(c2cnc(C)nc2)c2ccccc2Cl)n(C)c1=O] | B60 | ESI-MS m/z = 430.3 [M + H]+. Calculated MW: 429.86 |
| 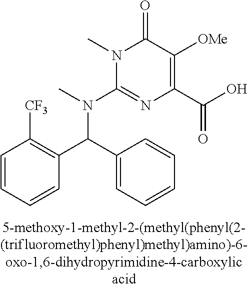 [SMILES: COc1c(C(=O)O)nc(N(C)C(c2ccccc2)c2ccccc2C(F)(F)F)n(C)c1=O] | B61 | ESI-MS m/z = 447.5 [M]+. Calculated MW: 447.41 |
| 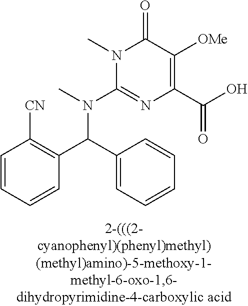 [SMILES: COc1c(C(=O)O)nc(N(C)C(c2ccccc2)c2ccccc2C#N)n(C)c1=O] | B62 | Sodium salt was taken on without further purification or analysis |
| 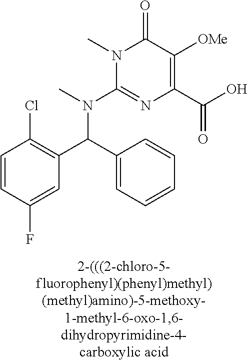 [SMILES: COc1c(C(=O)O)nc(N(C)C(c2ccccc2)c2cc(F)ccc2Cl)n(C)c1=O] | B63 | Sodium salt was taken on without further purification or analysis |
| 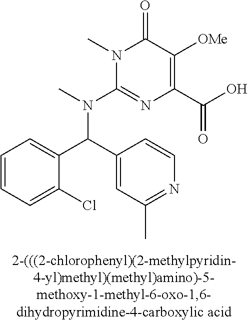 [SMILES: COc1c(C(=O)O)nc(N(C)C(c2ccnc(C)c2)c2ccccc2Cl)n(C)c1=O] | B64 | ESI-MS m/z = 429.4 [M + H]+. Calculated MW: 428.87 |
| 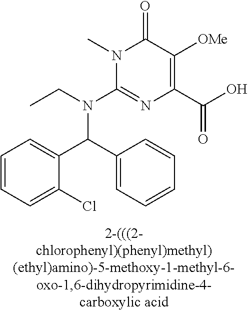 [SMILES: CCN(c1nc(C(=O)O)c(OC)c(=O)n1C)C(c1ccccc1)c1ccccc1Cl] | B65 | ESI-MS m/z = 428.3 [M + H]+. Calculated MW: 427.89 |
| 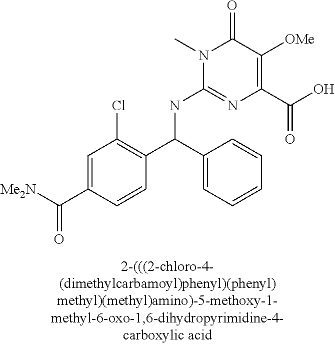 | B66 | ESI-MS m/z = 485.7 [M + H]+. Calculated MW: 484.94 |
| 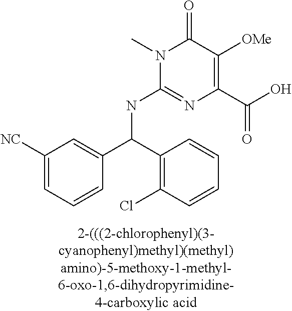 | B67 | ESI-MS m/z = 439.4 [M + H]+. Calculated MW: 438.87 |

### Synthesis of (2-[benzyl(ethyl)amino]-5-methoxy-1-methyl-6-oxopyrimidine-4-carboxylic acid), B13

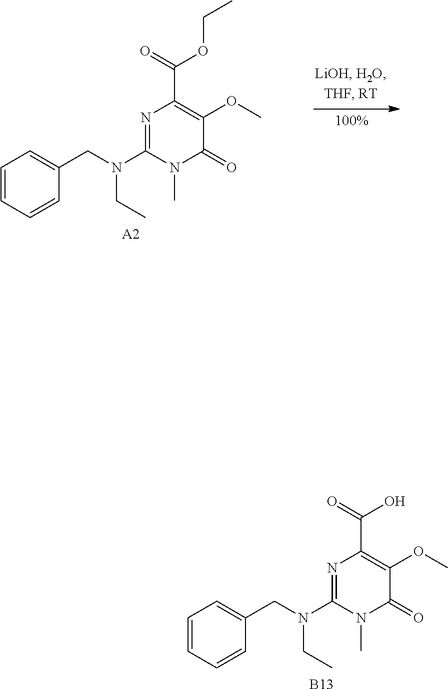

To a stirred mixture of (ethyl 2-[benzyl(ethyl)amino]-5-methoxy-1-methyl-6-oxopyrimi dine-4-carboxylate) A2 (1.00 g, 2.89 mmol, 1.00 equiv) in tetrahydrofuran (10.0 mL)/water (2.00 mL) was added Lithium hydroxide monohydrate (0.610 g, 14.5 mmol, 5.02 equiv) in portions at 0° C. under argon atmosphere. The resulting mixture was stirred for 2 h at room temperature under argon atmosphere. The mixture was acidified to pH 5 with hydrogen chloride (aq.). The resulting mixture was concentrated under reduced pressure to get 1 g of (2-[benzyl(ethyl)amino]-5-methoxy-1-methyl-6-oxopyrimidine-4-carboxylic acid) B13 (yield, 100%). The crude product was used in the next step directly without further purification. ESI−MS m/z=318.3 [M+H]⁺ Calculated MW: 317.3

The following intermediates were synthesized using similar conditions as those described in the step above along with appropriate starting materials.

| Structure | Intermediate | LCMS |
|---|---|---|
| 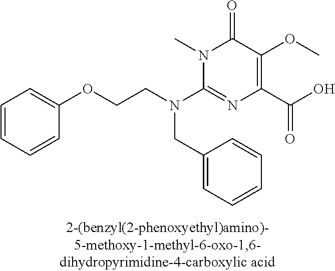 [SMILES: COc1c(C(=O)O)nc(N(CCOc2ccccc2)Cc2ccccc2)n(C)c1=O] | B14 | ESI-MS m/z = 410.2 [M + H]+. Calculated MW: 409.1 |
| 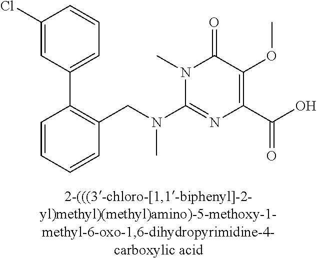 [SMILES: COc1c(C(=O)O)nc(N(C)Cc2ccccc2-c2cccc(Cl)c2)n(C)c1=O] | B15 | ESI-MS m/z = 414.2 [M + H]+. Calculated MW: 413.1 |
| 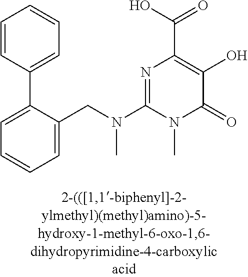 | B16 | ESI-MS m/z = 380.2 [M + H]+. Calculated MW: 379.2 |
| 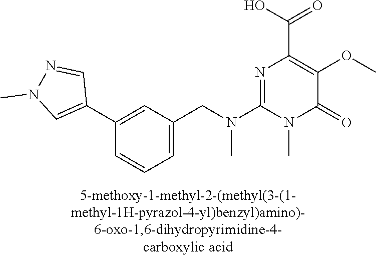 | B17 | ESI-MS m/z = 412.2 [M + H]+. Calculated MW: 411.2 |
| 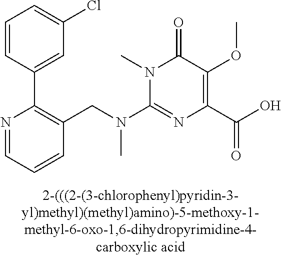 | B18 | ESI-MS m/z = 481.2 [M + H]+. Calculated MW: 480.2 |
| 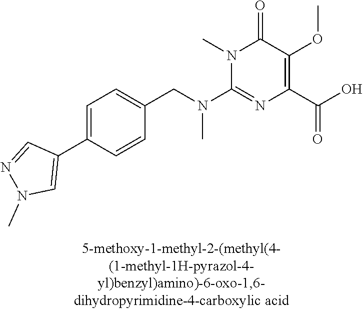 [SMILES: COc1c(C(=O)O)nc(N(C)Cc2ccc(-c3cnn(C)c3)cc2)n(C)c1=O] | B22 | ESI-MS m/z = 384.2 [M + H]+. Calculated MW: 383.2 |
| 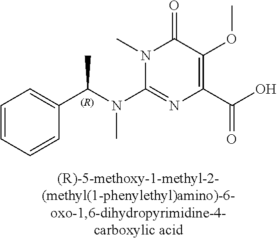 | B23 | ESI-MS m/z = 318.2 [M + H]+. Calculated MW: 317.1 |
| 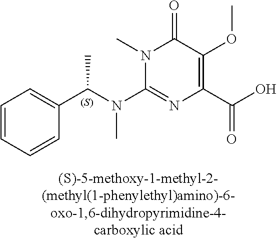 | B24 | ESI-MS m/z = 318.1 [M + H]+. Calculated MW: 317.1 |
| 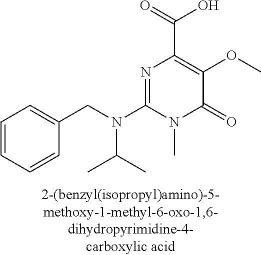 [SMILES: COc1c(C(=O)O)nc(N(Cc2ccccc2)C(C)C)n(C)c1=O] | B25 | ESI-MS m/z = 332.2 [M + H]+. Calculated MW: 331.1 |
| 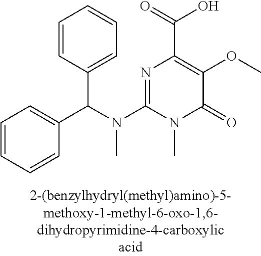 [SMILES: COc1c(C(=O)O)nc(N(C)C(c2ccccc2)c2ccccc2)n(C)c1=O] | B26 | ESI-MS m/Z = 380.4 [M + H]+. Calculated MW: 379.2 |
| 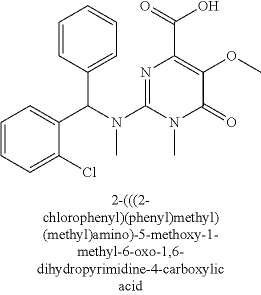 [SMILES: COc1c(C(=O)O)nc(N(C)C(c2ccccc2)c2ccccc2Cl)n(C)c1=O] | B27 From Isomer 1 | ESI-MS m/z = 414.2 [M + H]+. Calculated MW: 413.1 |
| 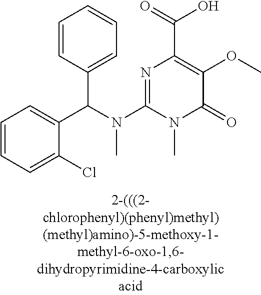 [SMILES: COc1c(C(=O)O)nc(N(C)C(c2ccccc2)c2ccccc2Cl)n(C)c1=O] | B28 From Isomer 2 | ESI-MS m/z = 414.1 [M + H]+. Calculated MW: 413.1 |
| 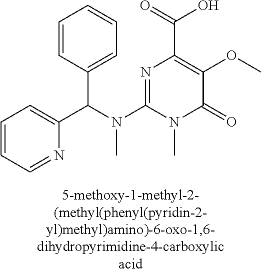 [SMILES: COc1c(C(=O)O)nc(N(C)C(c2ccccc2)c2ccccn2)n(C)c1=O] | B29 From Isomer 1 | ESI-MS m/z = 381.4 [M + H]+. Calculated MW: 380.2 |
| 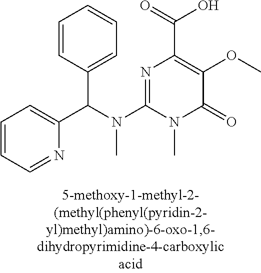 [SMILES: COc1c(C(=O)O)nc(N(C)C(c2ccccc2)c2ccccn2)n(C)c1=O] | B30 From Isomer 2 | ESI-MS m/z = 381.4 [M + H]+. Calculated MW: 380.2 |
| 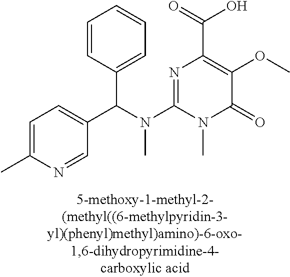 | B31 From Isomer 1 | ESI-MS m/z = 395.2 [M + H]+. Calculated MW: 394.2 |
| 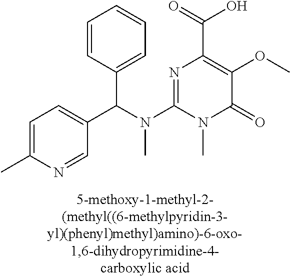 | B32 From Isomer 2 | ESI-MS m/z = 395.2 [M + H]+. Calculated MW: 394.2 |
| 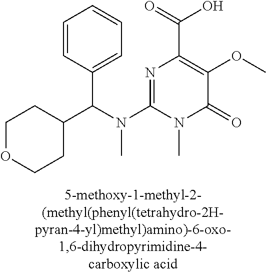 [SMILES: COc1c(C(=O)O)nc(N(C)C(c2ccccc2)C2CCOCC2)n(C)c1=O] | B33 From Isomer 1 | ESI-MS m/z = 388.4 [M + H]+. Calculated MW: 387.2 |
| 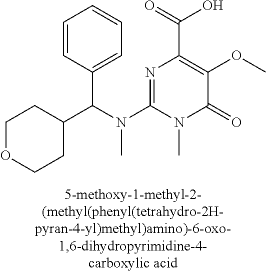 [SMILES: COc1c(C(=O)O)nc(N(C)C(c2ccccc2)C2CCOCC2)n(C)c1=O] | B34 From Isomer 2 | ESI-MS m/z = 388.4 [M + H]+. Calculated MW: 387.2 |
| 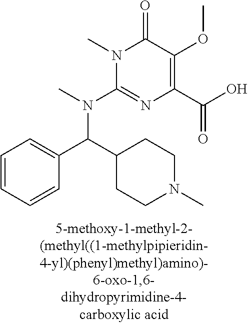 [SMILES: COc1c(C(=O)O)nc(N(C)C(c2ccccc2)C2CCN(C)CC2)n(C)c1=O] | B37 | ESI-MS m/z = 401.3 [M + H]+. Calculated MW: 400.2 |
| 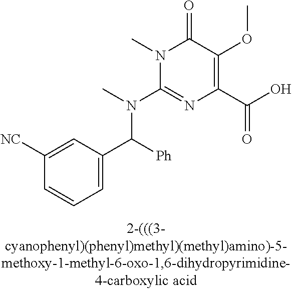 [SMILES: COc1c(C(=O)O)nc(N(C)C(c2ccccc2)c2cccc(C#N)c2)n(C)c1=O] | B38 From Isomer 1 | ESI-MS m/z = 405.3 [M + H]+. Calculated MW: 404.2 |
| 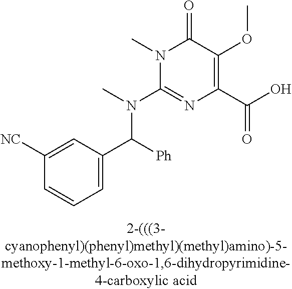 [SMILES: COc1c(C(=O)O)nc(N(C)C(c2ccccc2)c2cccc(C#N)c2)n(C)c1=O] | B39 From Isomer 2 | ESI-MS m/z = 405.4 [M + H]+. Calculated MW: 404.2 |
| 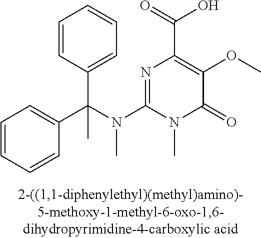 [SMILES: COc1c(C(=O)O)nc(N(C)C(C)(c2ccccc2)c2ccccc2)n(C)c1=O] | B40 | ESI-MS m/z = 394.4 [M + H]+. Calculated MW: 393.2 |
| 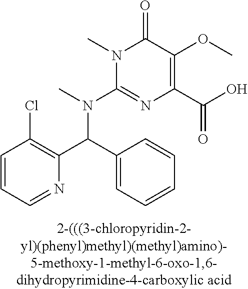 [SMILES: COc1c(C(=O)O)nc(N(C)C(c2ccccc2)c2ncccc2Cl)n(C)c1=O] | B41 | ESI-MS m/z = 415.1 [M + H]+. Calculated MW: 414.1 |
| 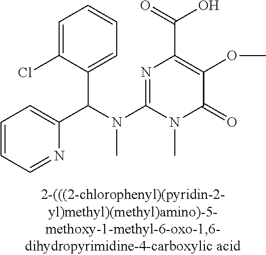 [SMILES: COc1c(C(=O)O)nc(N(C)C(c2ccccn2)c2ccccc2Cl)n(C)c1=O] | B43 | ESI-MS m/z = 415.2 [M + H]+. Calculated MW: 414.1 |
| 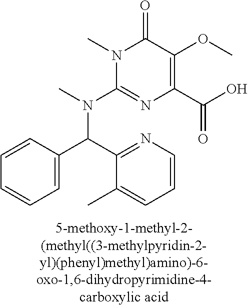 [SMILES: COc1c(C(=O)O)nc(N(C)C(c2ccccc2)c2ncccc2C)n(C)c1=O] | B44 | ESI-MS m/z = 395.4 [M + H]+. Calculated MW: 394.2 |
| 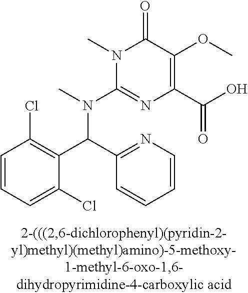 [SMILES: COc1c(C(=O)O)nc(N(C)C(c2ccccn2)c2c(Cl)cccc2Cl)n(C)c1=O] | B45 | ESI-MS m/z = 449.0 [M + H]+. Calculated MW: 448.1 |
| 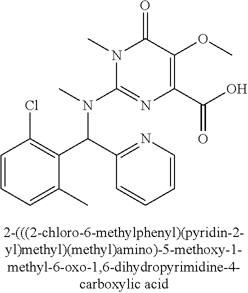 [SMILES: COc1c(C(=O)O)nc(N(C)C(c2ccccn2)c2c(C)cccc2Cl)n(C)c1=O] | B46 | ESI-MS m/z = 429.3 [M + H]+. Calculated MW: 428.1 |
| 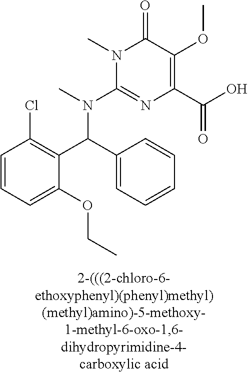 [SMILES: CCOc1cccc(Cl)c1C(c1ccccc1)N(C)c1nc(C(=O)O)c(OC)c(=O)n1C] | B48 | ESI-MS m/z = 458.1 [M + H]+. Calculated MW: 457.1 |
| 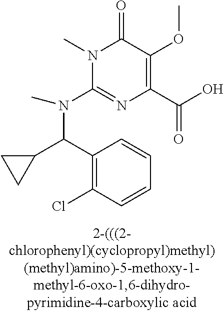 [SMILES: COc1c(C(=O)O)nc(N(C)C(c2ccccc2Cl)C2CC2)n(C)c1=O] | B49 From Isomer 1 | ESI-MS m/z = 378.1 [M + H]+. Calculated MW: 377.1 |
| 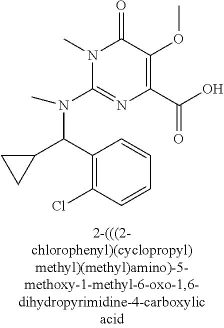 [SMILES: COc1c(C(=O)O)nc(N(C)C(c2ccccc2Cl)C2CC2)n(C)c1=O] | B50 From Isomer 2 | ESI-MS m/z = 378.1 [M + H]+. Calculated MW: 377.1 |
| 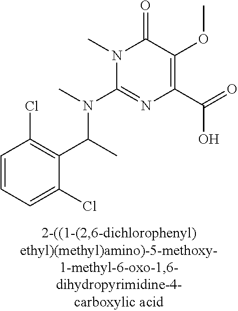 [SMILES: COc1c(C(=O)O)nc(N(C)C(C)c2c(Cl)cccc2Cl)n(C)c1=O] | B51 | ESI-MS m/z = 386.1 [M + H]+. Calculated MW: 385.1 |
| 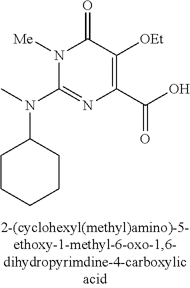 [SMILES: CCOc1c(C(=O)O)nc(N(C)C2CCCCC2)n(C)c1=O] | B69 | ESI-MS m/z = 310.3 [M + H]+. Calculated MW: 309.37 |
| 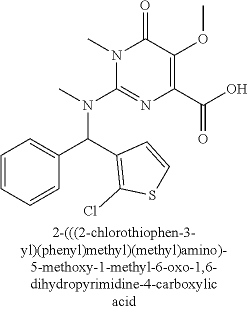 [SMILES: COc1c(C(=O)O)nc(N(C)C(c2ccccc2)c2ccsc2Cl)n(C)c1=O] | B70 | ESI-MS m/z = 419.8 [M + H]+. Calculated MW: 419.1 |

### 2-(dibenzylamino)-5-ethoxy-N-(isoxazol-4-yl)-1-methyl-6-oxo-1,6-dihydropyrimidine-4-carboxamide, C1

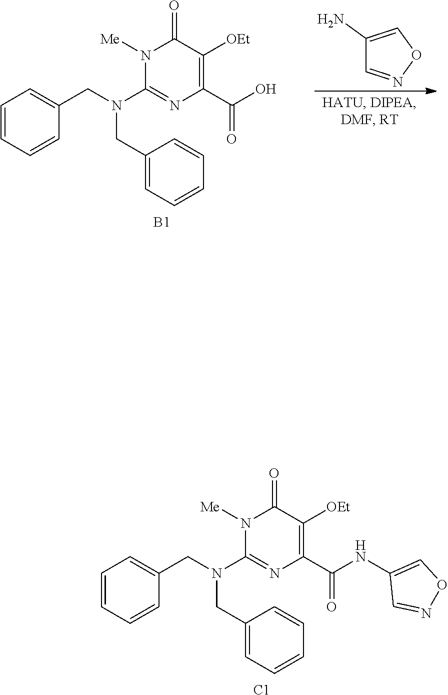

2-(dibenzylamino)-5-ethoxy-1-methyl-6-oxo-1,6-dihydropyrimidine-4-carboxylic acid, B1, (121 mg, 307 μmol) and HATU (233 mg, 614 μmol) were combined in DMF (3 mL) and stirred for 15 min before the sequential addition of 1,2-oxazol-4-amine hydrochloride (74.0 mg, 614 μmol) followed by triethylamine (128 μL, 921 mol). This mixture was then stirred at RT for 0.5 h. The reaction mixture was directly purified by reverse phase chromatography to give 107 mg of 2-(dibenzylamino)-5-ethoxy-N-(isoxazol-4-yl)-1-methyl-6-oxo-1,6-dihydropyrimidine-4-carboxamide, C1 in 76% yield. Calculated MW: 459.5 ESI−MS m/z=460.2 [M+H]⁺.

The following intermediates were synthesized using similar conditions as those described in the step above along with appropriate starting materials.

| Structure | Intermediate | LCMS |
|---|---|---|
| 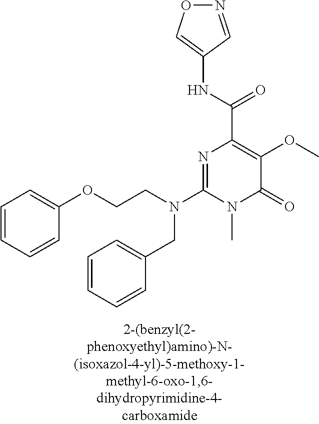 [SMILES: COc1c(C(=O)Nc2cnoc2)nc(N(CCOc2ccccc2)Cc2ccccc2)n(C)c1=O] | C2 | ESI-MS m/z = 476.2 [M + H]+. Calculated MW: 475.1 |
| 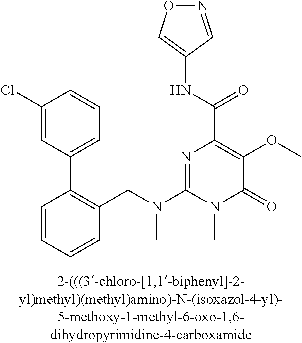 [SMILES: COc1c(C(=O)Nc2cnoc2)nc(N(C)Cc2ccccc2-c2cccc(Cl)c2)n(C)c1=O] | C3 | ESI-MS m/z = 480.4 [M + H]+. Calculated MW: 479.1 |
| 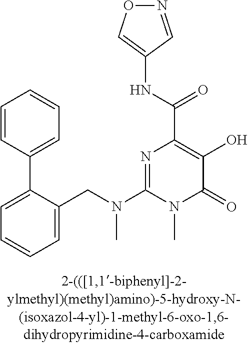 | C4 | ESI-MS m/z = 446.4 [M + H]+. Calculated MW: 445.2 |
| 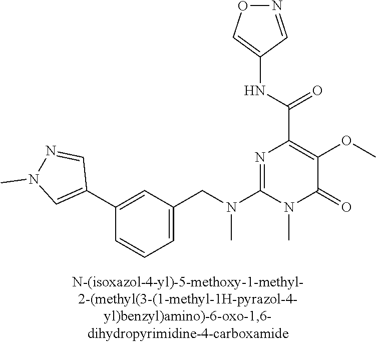 | C5 | ESI-MS m/z = 450.3 [M + H]+. Calculated MW: 449.2 |
| 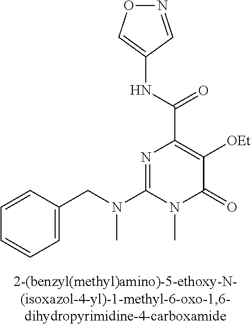 [SMILES: CCOc1c(C(=O)Nc2cnoc2)nc(N(C)Cc2ccccc2)n(C)c1=O] | C6 | ESI-MS m/z = 384.3 [M + H]+. Calculated MW: 383.3 |
| 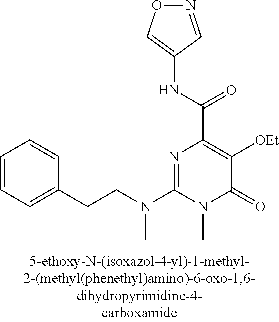 [SMILES: CCOc1c(C(=O)Nc2cnoc2)nc(N(C)CCc2ccccc2)n(C)c1=O] | C7 | ESI-MS m/z = 398.3 [M + H]+. Calculated MW: 397.4 |
| 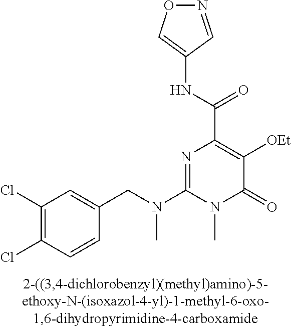 [SMILES: CCOc1c(C(=O)Nc2cnoc2)nc(N(C)Cc2ccc(Cl)c(Cl)c2)n(C)c1=O] | C8 | ESI-MS m/z = 452.5 [M + H]+. Calculated MW: 451.1 |
| 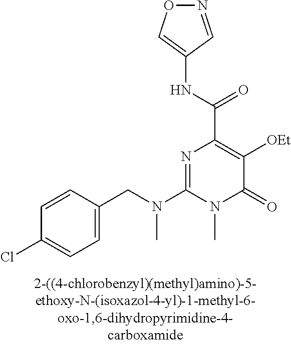 [SMILES: CCOc1c(C(=O)Nc2cnoc2)nc(N(C)Cc2ccc(Cl)cc2)n(C)c1=O] | C9 | ESI-MS m/z = 418.6 [M + H]+. Calculated MW: 417.9 |
| 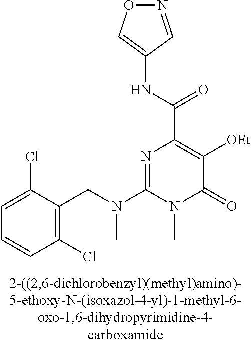 [SMILES: CCOc1c(C(=O)Nc2cnoc2)nc(N(C)Cc2c(Cl)cccc2Cl)n(C)c1=O] | C10 | ESI-MS m/z = 452.5 [M + H]+. Calculated MW: 451.1 |
| 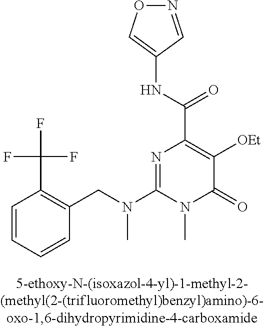 [SMILES: CCOc1c(C(=O)Nc2cnoc2)nc(N(C)Cc2ccccc2C(F)(F)F)n(C)c1=O] | C11 | ESI-MS m/z = 452.6 [M + H]+. Calculated MW: 451.4 |
| 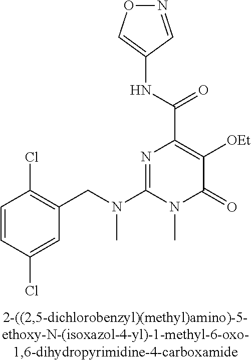 [SMILES: CCOc1c(C(=O)Nc2cnoc2)nc(N(C)Cc2cc(Cl)ccc2Cl)n(C)c1=O] | C12 | ESI-MS m/z = 452.0 [M + H]+. Calculated MW: 451.1 |
| 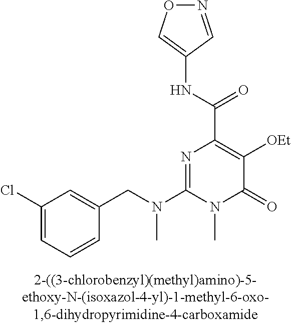 [SMILES: CCOc1c(C(=O)Nc2cnoc2)nc(N(C)Cc2cccc(Cl)c2)n(C)c1=O] | C13 | ESI-MS m/z = 418.5 [M + H]+. Calculated MW: 417.9 |
| 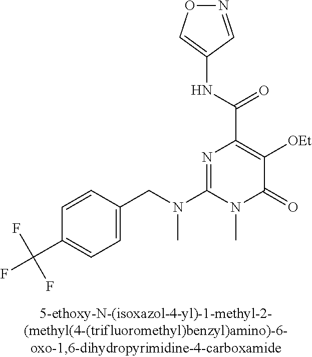 [SMILES: CCOc1c(C(=O)Nc2cnoc2)nc(N(C)Cc2ccc(C(F)(F)F)cc2)n(C)c1=O] | C14 | ESI-MS m/z = 452.3 [M + H]+. Calculated MW: 451.4 |
| 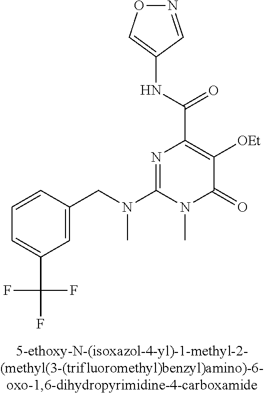 [SMILES: CCOc1c(C(=O)Nc2cnoc2)nc(N(C)Cc2cccc(C(F)(F)F)c2)n(C)c1=O] | C15 | ESI-MS m/z = 452.6 [M + H]+. Calculated MW: 451.4 |
| 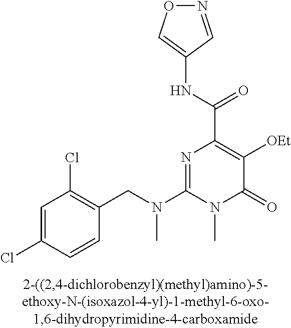 [SMILES: CCOc1c(C(=O)Nc2cnoc2)nc(N(C)Cc2ccc(Cl)cc2Cl)n(C)c1=O] | C16 | ESI-MS m/z = 452.5 [M + H]+. Calculated MW: 451.1 |
| 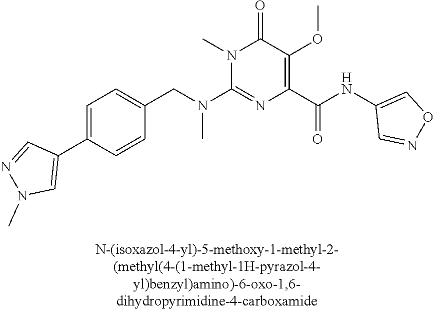 [SMILES: COc1c(C(=O)Nc2cnoc2)nc(N(C)Cc2ccc(-c3cnn(C)c3)cc2)n(C)c1=O] | C25 | ESI-MS m/z = 450.2 [M + H]+. Calculated MW: 449.2 |
| 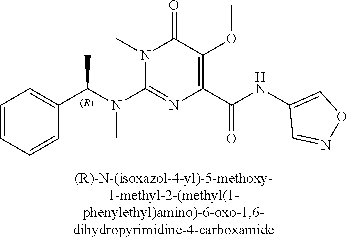 | C26 | ESI-MS m/z = 384.1 [M + H]+. Calculated MW: 383.2 |
| 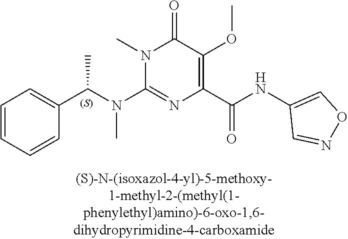 | C27 | ESI-MS m/z = 384.1 [M + H]+. Calculated MW: 383.2 |
| 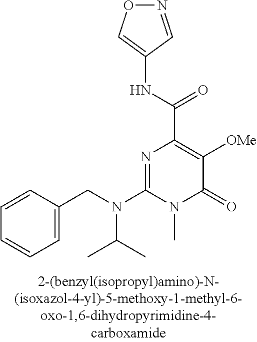 [SMILES: COc1c(C(=O)Nc2cnoc2)nc(N(Cc2ccccc2)C(C)C)n(C)c1=O] | C28 | ESI-MS m/z = 398.2 [M + H]+. Calculated MW: 397.2 |
| 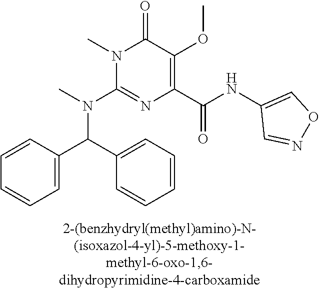 [SMILES: COc1c(C(=O)Nc2cnoc2)nc(N(C)C(c2ccccc2)c2ccccc2)n(C)c1=O] | C29 | ESI-MS m/z = 446.4 [M + H]+. Calculated MW: 445.2 |
| 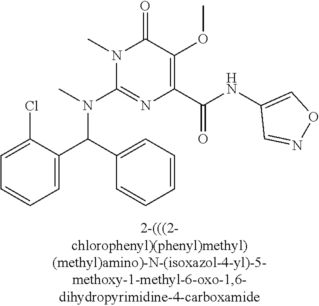 [SMILES: COc1c(C(=O)Nc2cnoc2)nc(N(C)C(c2ccccc2)c2ccccc2Cl)n(C)c1=O] | C39 From Isomer 1 | ESI-MS m/z = 480.3 [M + H]+. Calculated MW: 479.1 |
| 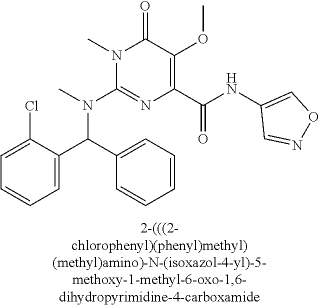 [SMILES: COc1c(C(=O)Nc2cnoc2)nc(N(C)C(c2ccccc2)c2ccccc2Cl)n(C)c1=O] | C40 From Isomer 2 | ESI-MS m/z = 480.1 [M + H]+. Calculated MW: 479.1 |
| 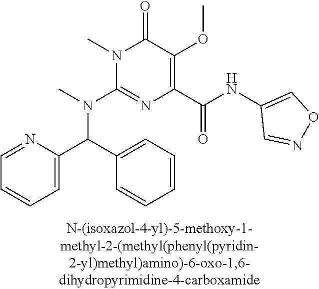 [SMILES: COc1c(C(=O)Nc2cnoc2)nc(N(C)C(c2ccccc2)c2ccccn2)n(C)c1=O] | C41 From Isomer 1 | ESI-MS m/z = 447.1 [M + H]+. Calculated MW: 446.2 |
| 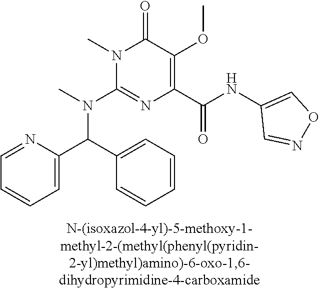 [SMILES: COc1c(C(=O)Nc2cnoc2)nc(N(C)C(c2ccccc2)c2ccccn2)n(C)c1=O] | C42 From Isomer 2 | ESI-MS m/z = 447.1 [M + H]+. Calculated MW: 446.2 |
| 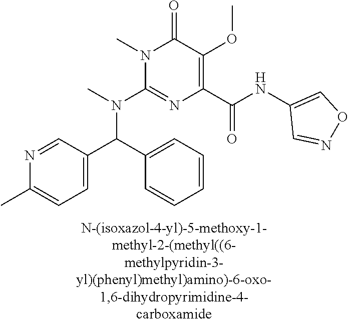 [SMILES: COc1c(C(=O)Nc2cnoc2)nc(N(C)C(c2ccccc2)c2ccc(C)nc2)n(C)c1=O] | C43 From Isomer 1 | ESI-MS m/z = 461.1 [M + H]+. Calculated MW: 460.2 |
| 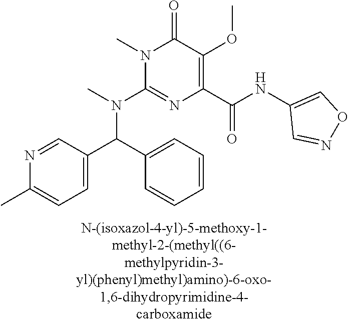 [SMILES: COc1c(C(=O)Nc2cnoc2)nc(N(C)C(c2ccccc2)c2ccc(C)nc2)n(C)c1=O] | C44 From Isomer 2 | ESI-MS m/z = 461.1 [M + H]+. Calculated MW: 460.2 |
| 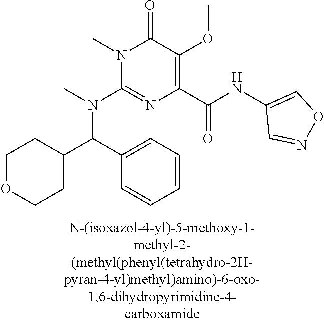 [SMILES: COc1c(C(=O)Nc2cnoc2)nc(N(C)C(c2ccccc2)C2CCOCC2)n(C)c1=O] | C45 From Isomer 1 | ESI-MS m/z = 454.4 [M + H]+. Calculated MW: 453.2 |
| 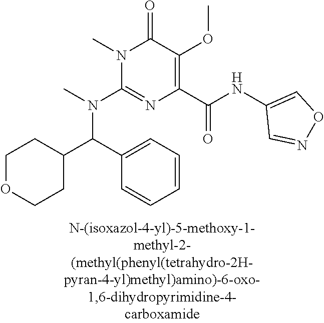 [SMILES: COc1c(C(=O)Nc2cnoc2)nc(N(C)C(c2ccccc2)C2CCOCC2)n(C)c1=O] | C46 From Isomer 2 | ESI-MS m/z = 454.4 [M + H]+. Calculated MW: 453.2 |
| 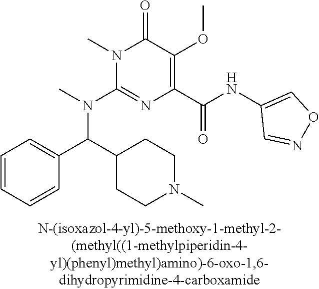 [SMILES: COc1c(C(=O)Nc2cnoc2)nc(N(C)C(c2ccccc2)C2CCN(C)CC2)n(C)c1=O] | C49 | ESI-MS m/z = 467.4 [M + H]+. Calculated MW: 466.2 |
| 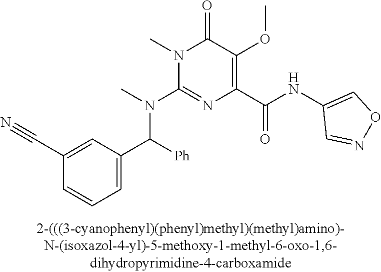 [SMILES: COc1c(C(=O)Nc2cnoc2)nc(N(C)C(c2ccccc2)c2cccc(C#N)c2)n(C)c1=O] | C50 From Isomer 1 | ESI-MS m/z = 471.4 [M + H]+. Calculated MW: 470.2 |
| 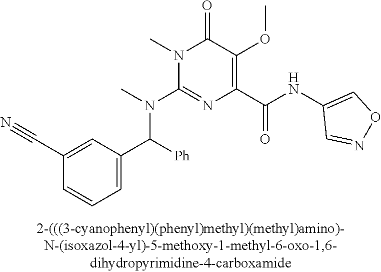 [SMILES: COc1c(C(=O)Nc2cnoc2)nc(N(C)C(c2ccccc2)c2cccc(C#N)c2)n(C)c1=O] | C51 From Isomer 2 | ESI-MS m/z = 471.4 [M + H]+. Calculated MW: 470.2 |
| 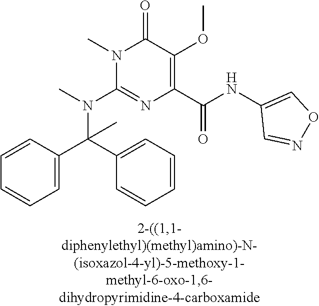 [SMILES: COc1c(C(=O)Nc2cnoc2)nc(N(C)C(C)(c2ccccc2)c2ccccc2)n(C)c1=O] | C52 | ESI-MS m/z = 460.1 [M + H]+. Calculated MW: 459.2 |
| 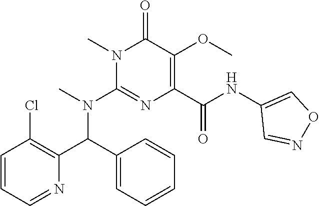 [SMILES: COc1c(C(=O)Nc2cnoc2)nc(N(C)C(c2ccccc2)c2ncccc2Cl)n(C)c1=O] | C52 | ESI-MS m/z = 481.3 [M + H]+. Calculated MW: 480.1 |
| 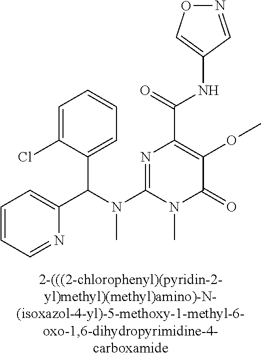 [SMILES: COc1c(C(=O)Nc2cnoc2)nc(N(C)C(c2ccccn2)c2ccccc2Cl)n(C)c1=O] | C54 | ESI-MS m/z = 481.4 [M + H]+. Calculated MW: 480.1 |
| 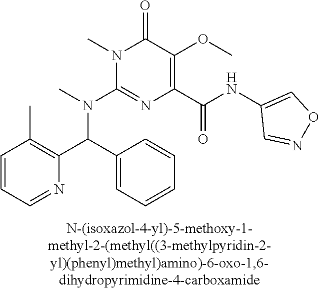 [SMILES: COc1c(C(=O)Nc2cnoc2)nc(N(C)C(c2ccccc2)c2ncccc2C)n(C)c1=O] | C55 | ESI-MS m/z = 461.4 [M + H]+. Calculated MW: 460.2 |
| 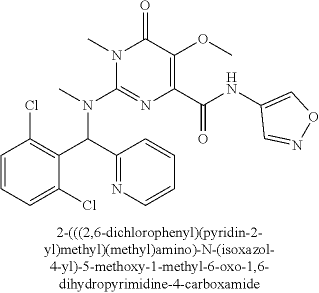 [SMILES: COc1c(C(=O)Nc2cnoc2)nc(N(C)C(c2ccccn2)c2c(Cl)cccc2Cl)n(C)c1=O] | C56 | ESI-MS m/z = 515.3 [M + H]+. Calculated MW: 514.1 |
| 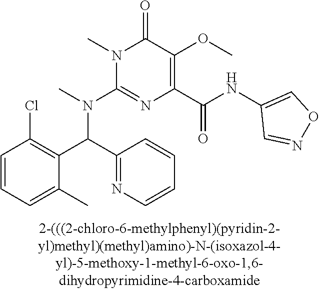 [SMILES: COc1c(C(=O)Nc2cnoc2)nc(N(C)C(c2ccccn2)c2c(C)cccc2Cl)n(C)c1=O] | C57 | ESI-MS m/z = 495.0 [M + H]+. Calculated MW: 494.2 |
| 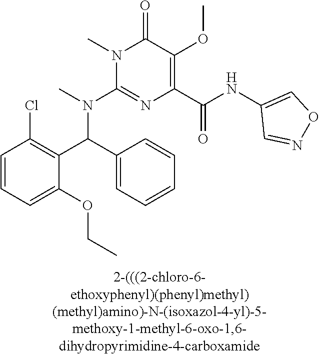 [SMILES: CCOc1cccc(Cl)c1C(c1ccccc1)N(C)c1nc(C(=O)Nc2cnoc2)c(OC)c(=O)n1C] | C59 | ESI-MS m/z = 524.2 [M + H]+. Calculated MW: 523.2 |
| 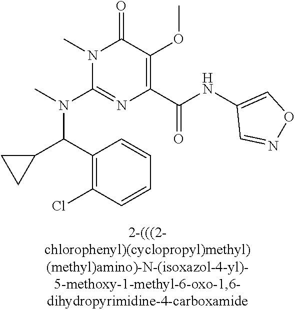 [SMILES: COc1c(C(=O)Nc2cnoc2)nc(N(C)C(c2ccccc2Cl)C2CC2)n(C)c1=O] | C60 From Isomer 1 | ESI-MS m/z = 444.1 [M + H]+. Calculated MW: 443.1 |
| 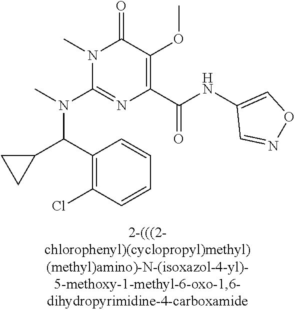 [SMILES: COc1c(C(=O)Nc2cnoc2)nc(N(C)C(c2ccccc2Cl)C2CC2)n(C)c1=O] | C61 From Isomer 2 | ESI-MS m/z = 444.1 [M + H]+. Calculated MW: 443.1 |
| 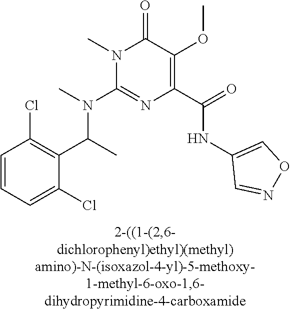 [SMILES: COc1c(C(=O)Nc2cnoc2)nc(N(C)C(C)c2c(Cl)cccc2Cl)n(C)c1=O] | C62 | ESI-MS m/z = 452.0 [M + H]+. Calculated MW: 451.1 |
| 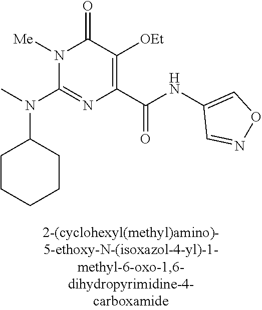 [SMILES: CCOc1c(C(=O)Nc2cnoc2)nc(N(C)C2CCCCC2)n(C)c1=O] | C63 | ESI-MS m/z = 376.3 [M + H]+. Calculated MW: 375.43 |
| 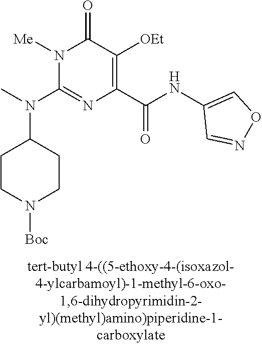 [SMILES: CCOc1c(C(=O)Nc2cnoc2)nc(N(C)C2CCN(C(=O)OC(C)(C)C)CC2)n(C)c1=O] | C64 | ESI-MS m/z = 477.6 [M + H]+. Calculated MW: 476.53 |
| 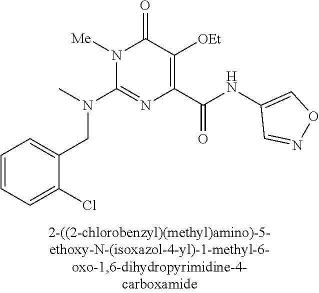 [SMILES: CCOc1c(C(=O)Nc2cnoc2)nc(N(C)Cc2ccccc2Cl)n(C)c1=O] | C65 | ESI-MS m/z = 418.6 [M + H]+. Calculated MW: 417.85 |
| 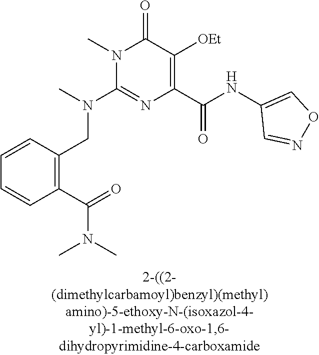 [SMILES: CCOc1c(C(=O)Nc2cnoc2)nc(N(C)Cc2ccccc2C(=O)N(C)C)n(C)c1=O] | C66 | ESI-MS m/z = 455.36 [M + H]+. Calculated MW: 454.49 |
| 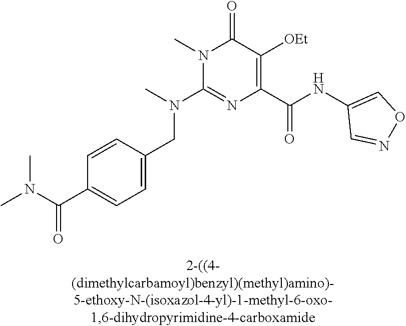 [SMILES: CCOc1c(C(=O)Nc2cnoc2)nc(N(C)Cc2ccc(C(=O)N(C)C)cc2)n(C)c1=O] | C67 | ESI-MS m/z = 455.34 [M + H]+. Calculated MW: 454.49 |
| 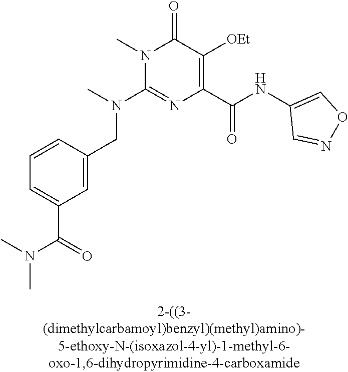 [SMILES: CCOc1c(C(=O)Nc2cnoc2)nc(N(C)Cc2cccc(C(=O)N(C)C)c2)n(C)c1=O] | C68 | ESI-MS m/z = 455.41 [M + H]+. Calculated MW: 454.49 |
| 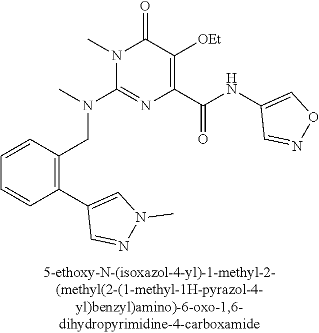 [SMILES: CCOc1c(C(=O)Nc2cnoc2)nc(N(C)Cc2ccccc2-c2cnn(C)c2)n(C)c1=O] | C69 | ESI-MS m/z = 464.34 [M + H]+. Calculated MW: 463.50 |
| 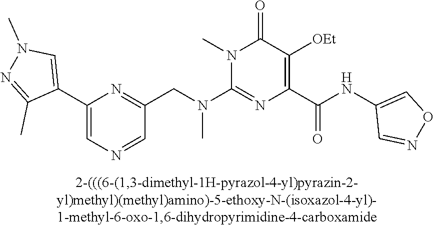 [SMILES: CCOc1c(C(=O)Nc2cnoc2)nc(N(C)Cc2cncc(-c3cn(C)nc3C)n2)n(C)c1=O] | C69 | ESI-MS m/z = 480.4 [M + H]+. Calculated MW: 479.50 |
| 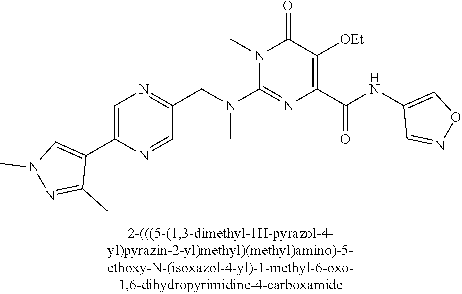 [SMILES: CCOc1c(C(=O)Nc2cnoc2)nc(N(C)Cc2cnc(-c3cn(C)nc3C)cn2)n(C)c1=O] | C70 | ESI-MS m/z = 480.41 [M + H]+. Calculated MW: 479.50 |
| 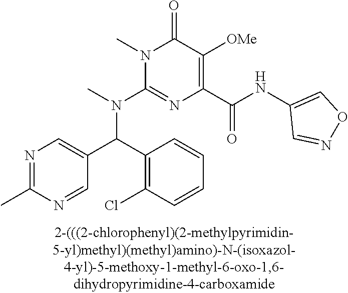 [SMILES: COc1c(C(=O)Nc2cnoc2)nc(N(C)C(c2cnc(C)nc2)c2ccccc2Cl)n(C)c1=O] | C71 | ESI-MS m/z = 496.72 [M + H]+. Calculated MW: 495.92 |
| 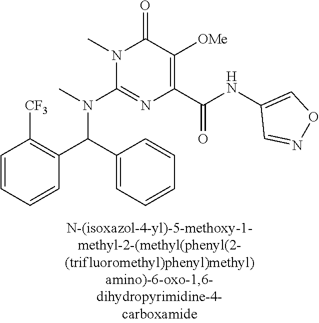 [SMILES: COc1c(C(=O)Nc2cnoc2)nc(N(C)C(c2ccccc2)c2ccccc2C(F)(F)F)n(C)c1=O] | C72 | ESI-MS m/z = 514.3 [M + H]+. Calculated MW: 513.48 |
| 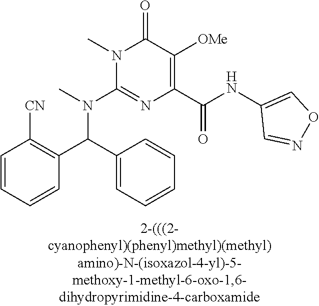 [SMILES: COc1c(C(=O)Nc2cnoc2)nc(N(C)C(c2ccccc2)c2ccccc2C#N)n(C)c1=O] | C73 | ESI-MS m/z = 471.39 [M + H]+. Calculated MW: 470.49 |
| 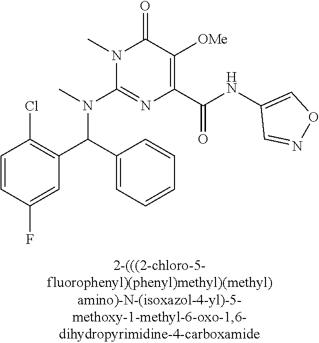 [SMILES: COc1c(C(=O)Nc2cnoc2)nc(N(C)C(c2ccccc2)c2cc(F)ccc2Cl)n(C)c1=O] | C74 | ESI-MS m/z = 498.4 [M + H]+. Calculated MW: 497.91 |
| 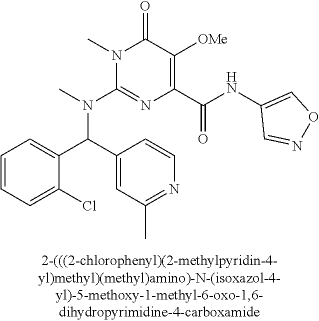 [SMILES: COc1c(C(=O)Nc2cnoc2)nc(N(C)C(c2ccnc(C)c2)c2ccccc2Cl)n(C)c1=O] | C75 | ESI-MS m/z = 495.81 [M + H]+. Calculated MW: 494.94 |
| 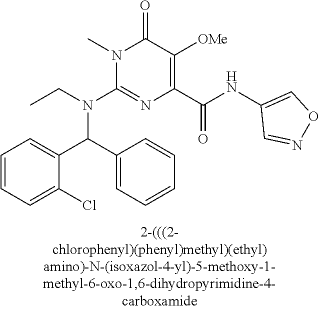 [SMILES: CCN(c1nc(C(=O)Nc2cnoc2)c(OC)c(=O)n1C)C(c1ccccc1)c1ccccc1Cl] | C76 | ESI-MS m/z = 494.52 [M + H]+. Calculated MW: 493.95 |
| 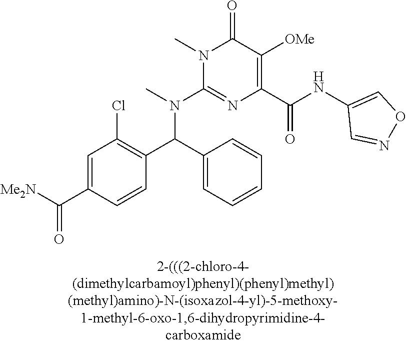 [SMILES: COc1c(C(=O)Nc2cnoc2)nc(N(C)C(c2ccccc2)c2ccc(C(=O)N(C)C)cc2Cl)n(C)c1=O] | C77 | ESI-MS m/z = 551.9 [M + H]+. Calculated MW: 550.17 |
| 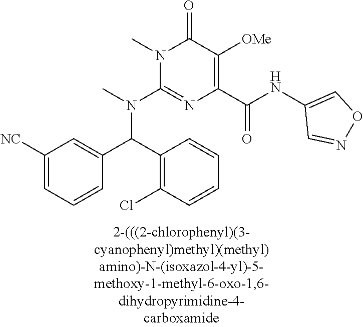 [SMILES: COc1c(C(=O)Nc2cnoc2)nc(N(C)C(c2cccc(C#N)c2)c2ccccc2Cl)n(C)c1=O] | C78 | ESI-MS m/z = 505.4 [M + H]+. Calculated MW: 504.93 |
| 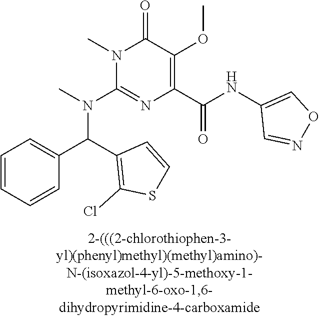 [SMILES: COc1c(C(=O)Nc2cnoc2)nc(N(C)C(c2ccccc2)c2ccsc2Cl)n(C)c1=O] | C79 | ESI-MS m/z = 486.2 [M + H]+. Calculated MW: 485.1 |

Chiral separation of O-methylhydroxypyrimidinone Amides

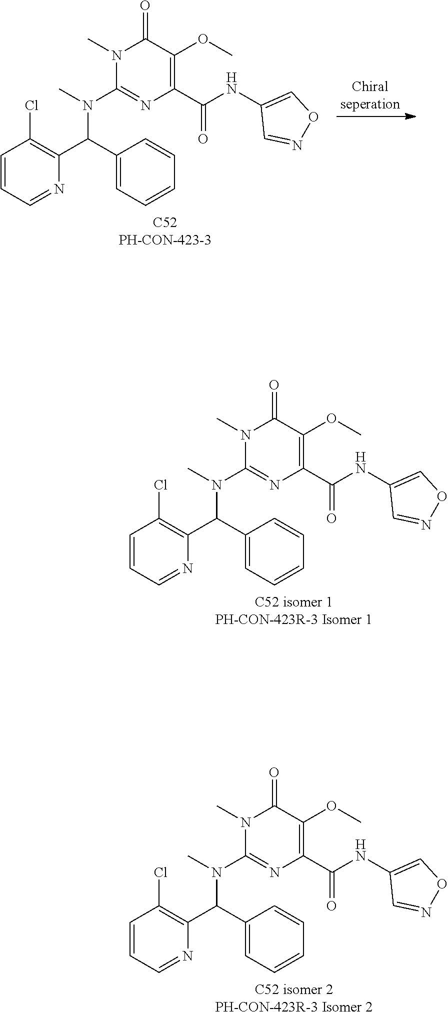

The two enantiomers of compound C₅₂ were separated via chiral HPLC using a CHIRAL ART Cellulose-SB column (0.46*10 cm, 3 um) using hexanes (0.1% diethylamine) and ethanol in a 70 to 30 ratio as the eluent at a flow rate of 1.0 mL/min and ambient temperature. C52 isomer 1 eluted at 3.98 min (eutomer) and C52 (distomer) eluted at 4.71 min.

The following intermediates were separated using similar conditions as those described above.

| | | Chiral Separation | Retention |
|---|---|---|---|
| Structure | Intermediate | Conditions | Time |
| 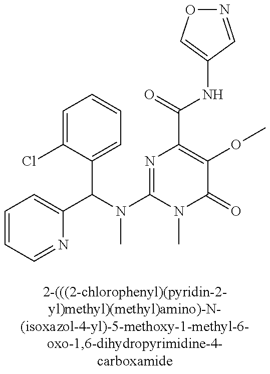 [SMILES: COc1c(C(=O)Nc2cnoc2)nc(N(C)C(c2ccccn2)c2ccccc2Cl)n(C)c1=O] | C54-2 Isomer 1 | CHIRALPAK IA, 0.46*10 cm, 5.0 um; Mobile Phase A: CO2, Mobile Phase B: MeOH(0.1% DEA)- SFC; Flow rate: 2 mL/min | 2.96 min |
| 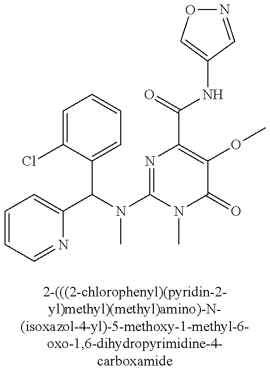 [SMILES: COc1c(C(=O)Nc2cnoc2)nc(N(C)C(c2ccccn2)c2ccccc2Cl)n(C)c1=O] | C54-2 Isomer 2 | CHIRALPAK IA, 0.46*10 cm, 5.0 um; Mobile Phase A: CO2, Mobile Phase B: MeOH(0.1% DEA)- SFC; Flow rate: 2 mL/min | 3.22 min |
| 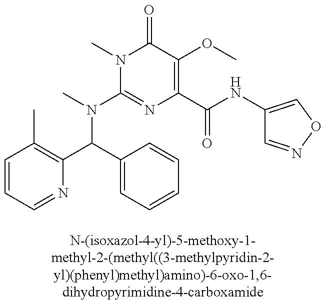 [SMILES: COc1c(C(=O)Nc2cnoc2)nc(N(C)C(c2ccccc2)c2ncccc2C)n(C)c1=O] | C55-2 Isomer 1 | CHIRAL ART Cellulose-SB, 0.46 * 10 cm, 3 um; Mobile Phase: Hex(0.1% DEA):EtOH = 50:50-HPLC; Flow rate: 1 mL/min | 2.23 min |
| 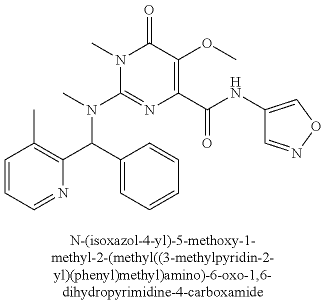 [SMILES: COc1c(C(=O)Nc2cnoc2)nc(N(C)C(c2ccccc2)c2ncccc2C)n(C)c1=O] | C55-2 Isomer 2 | CHIRAL ART Cellulose-SB, 0.46 * 10 cm, 3 um; Mobile Phase: Hex(0.1% DEA):EtOH = 50:50-HPLC; Flow rate: 1 mL/min | 2.59 min |
| 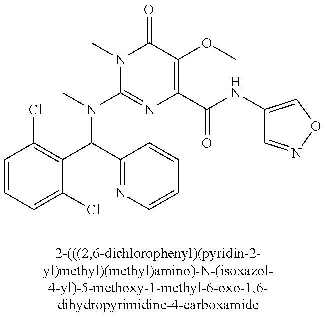 [SMILES: COc1c(C(=O)Nc2cnoc2)nc(N(C)C(c2ccccn2)c2c(Cl)cccc2Cl)n(C)c1=O] | C56-2 Isomer 1 | Amylose-C Neo, 0.46 * 10 cm(3 um); Mobile Phase: Hex(0.1% DEA):EtOH = 50:50-HPLC; Flow rate: 1 mL/min | 2.98 min |
| 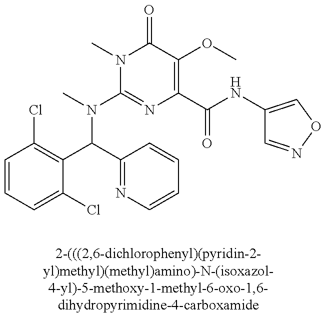 [SMILES: COc1c(C(=O)Nc2cnoc2)nc(N(C)C(c2ccccn2)c2c(Cl)cccc2Cl)n(C)c1=O] | C56-2 Isomer 2 | Amylose-C Neo, 0.46 * 10 cm(3 um); Mobile Phase: Hex(0.1% DEA):EtOH = 50:50-HPLC; Flow rate: 1 mL/min | 4.40 min |
| 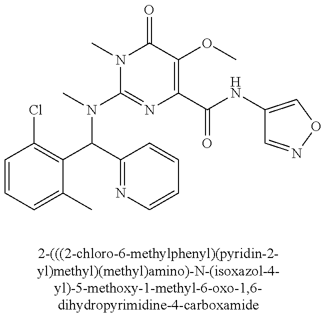 [SMILES: COc1c(C(=O)Nc2cnoc2)nc(N(C)C(c2ccccn2)c2c(C)cccc2Cl)n(C)c1=O] | C57-2 Isomer 1 | CHIRAL ART Cellulose-SB, 0.46*10 cm, 3 um; Mobile phase: Hex(10 mM NH3):EtOH = 50:50- HPLC; Flow rate: 1 mL/min | 3.57 min |
| 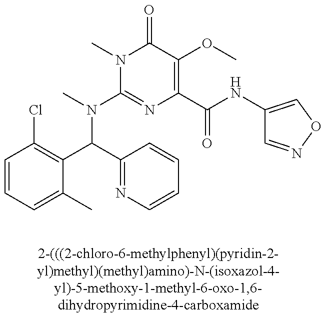 [SMILES: COc1c(C(=O)Nc2cnoc2)nc(N(C)C(c2ccccn2)c2c(C)cccc2Cl)n(C)c1=O] | C57-2 Isomer 2 | CHIRAL ART Cellulose-SB, 0.46*10 cm, 3 um; Mobile phase:Hex (10 mM NH3):EtOH = 50:50- HPLC; Flow rate: 1 mL/min | 4.27 min |
| 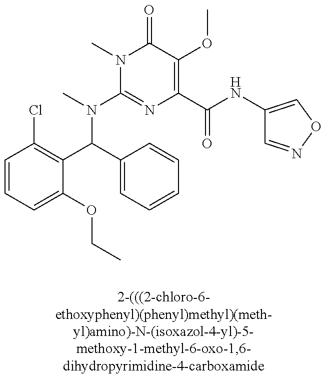 [SMILES: CCOc1cccc(Cl)c1C(c1ccccc1)N(C)c1nc(C(=O)Nc2cnoc2)c(OC)c(=O)n1C] | C59-2 Isomer 1 distomer | CHIRALPAK IA- U, 3.0*50 mm, 1.6 um; Mobile Phase: Hex(0.1% DEA):IPA = 70:30; Flow rate: 1 mL/min | 1.61 min |
| 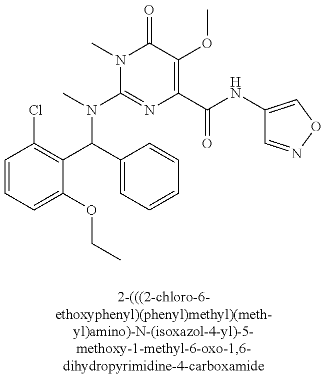 [SMILES: CCOc1cccc(Cl)c1C(c1ccccc1)N(C)c1nc(C(=O)Nc2cnoc2)c(OC)c(=O)n1C] | C59-2 Isomer 2 eutomer | CHIRALPAK IA- U, 3.0*50 mm, 1.6 um; Mobile Phase: Hex(0.1% DEA):IPA = 70:30, Flow rate: 1 mL/min | 2.44 min |
| 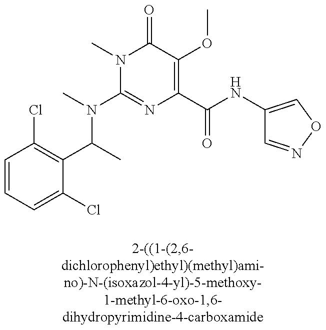 [SMILES: COc1c(C(=O)Nc2cnoc2)nc(N(C)C(C)c2c(Cl)cccc2Cl)n(C)c1=O] | C62-2 Isomer 1 | CHIRAL ART Cellulose-SB, 0.46 * 10 cm, 3 um; Mobile Phase: MtBE(0.1% FA):EtOH = 50:50-HPLC; Flow rate: 1 mL/min | 1.89 min |
| 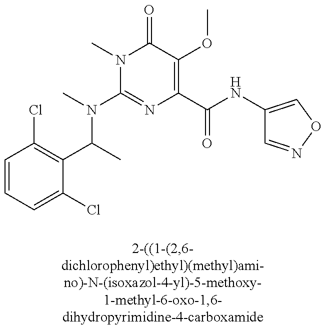 [SMILES: COc1c(C(=O)Nc2cnoc2)nc(N(C)C(C)c2c(Cl)cccc2Cl)n(C)c1=O] | C62-2 Isomer 2 | CHIRAL ART Cellulose-SB, 0.46 * 10 cm, 3 um; Mobile Phase: MtBe(0.1% FA):EtOH = 50:50-HPLC; Flow rate: 1 mL/min | 2.11 min |
| 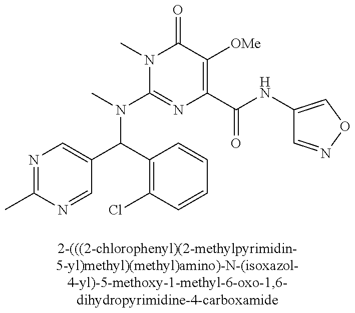 [SMILES: COc1c(C(=O)Nc2cnoc2)nc(N(C)C(c2cnc(C)nc2)c2ccccc2Cl)n(C)c1=O] | C71-2 Isomer 1 | CHIRALPAK IH; 25% IPA:MeOH (50:50) in Hexanes + 0.1% DEA) Flow rate: 20 ml/min | 9.55 min |
| 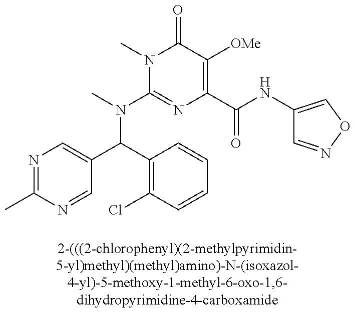 [SMILES: COc1c(C(=O)Nc2cnoc2)nc(N(C)C(c2cnc(C)nc2)c2ccccc2Cl)n(C)c1=O] | C71-2 Isomer 2 | CHIRALPAK IH; 25 IPA:MeOH (50:50) in Hexanes + 0.1% DEA) Flow rate: 20 ml/min | 10.43 min |
| 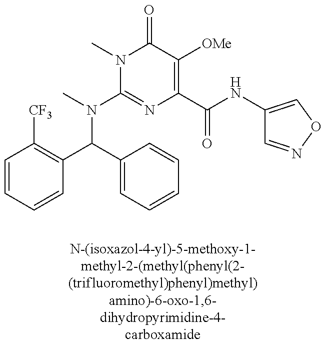 [SMILES: COc1c(C(=O)Nc2cnoc2)nc(N(C)C(c2ccccc2)c2ccccc2C(F)(F)F)n(C)c1=O] | C72-2 Isomer 1 | Chiral SFC (CHIRALPAK IH; 15% (IPA:ACN; 70:30) in hexanes + 0.1% DEA) Flow rate: 16 ml/min | 7.61 min |
| 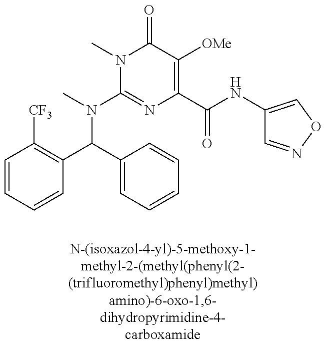 [SMILES: COc1c(C(=O)Nc2cnoc2)nc(N(C)C(c2ccccc2)c2ccccc2C(F)(F)F)n(C)c1=O] | C72-2 Isomer 2 | Chiral SFC (CHIRALPAK IH; 15% (IPA:ACN; 70:30) in hexanes + 0.1% DEA) Flow rate: 16 ml/min | 8.07 min |
| 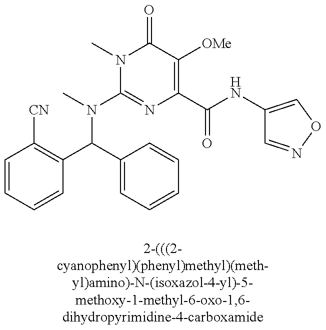 [SMILES: COc1c(C(=O)Nc2cnoc2)nc(N(C)C(c2ccccc2)c2ccccc2C#N)n(C)c1=O] | C73-2 Isomer 1 | Chiral SFC (CHIRALPAK IB- N; 25% IPA:MeOH (50:50) in Hexanes + 0.1% DEA) Flow rate: 18 ml/min | 5.03 min |
| 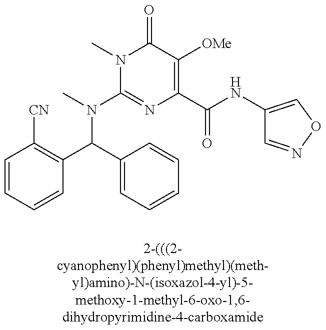 [SMILES: COc1c(C(=O)Nc2cnoc2)nc(N(C)C(c2ccccc2)c2ccccc2C#N)n(C)c1=O] | C73-2 Isomer 2 | Chiral SFC (CHIRALPAK IB- N; 25% IPA: MeOH (50:50) in Hexanes + 0.1% DEA) Flow rate: 18 ml/min | 6.21 min |
| 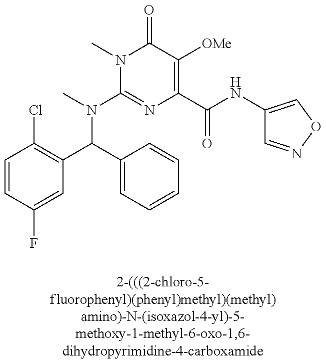 [SMILES: COc1c(C(=O)Nc2cnoc2)nc(N(C)C(c2ccccc2)c2cc(F)ccc2Cl)n(C)c1=O] | C74-2 Isomer 1 | Chiral SFC (CHIRALPAK AD- H; 20% (MeOH:ACN; 50:50) in Liquid CO₂ + 0.1% DEA) Flow rate: 80 ml/min | 4.04 min |
| 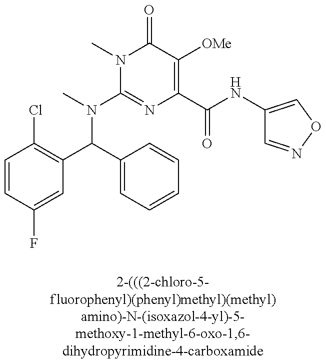 [SMILES: COc1c(C(=O)Nc2cnoc2)nc(N(C)C(c2ccccc2)c2cc(F)ccc2Cl)n(C)c1=O] | C74-2 Isomer 2 | Chiral SFC (CHIRALPAK AD- H; 20% MeOH:ACN; 50:50) in Liquid CO₂ + 0.1% DEA) Flow rate: 80 ml/min | 4.63 min |
| 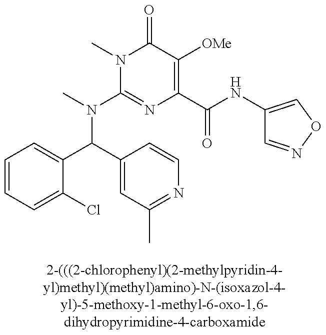 [SMILES: COc1c(C(=O)Nc2cnoc2)nc(N(C)C(c2ccnc(C)c2)c2ccccc2Cl)n(C)c1=O] | C75-2 Isomer 1 | Chiral SFC (CHIRALCEL OJ- H; 15% (MeOH) in Liquid CO₂ + 0.1% DEA) Chiral SFC (CHIRALCEL OX- H; 40% (IPA:MeOH; 50:50) in Hexanes + 0.1% DEA) Flow rate: 18 ml/min | 3.73 min |
| 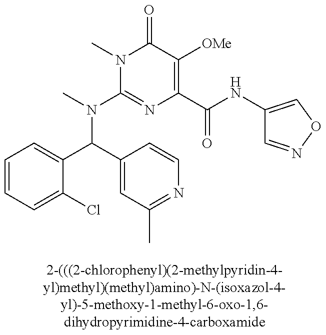 [SMILES: COc1c(C(=O)Nc2cnoc2)nc(N(C)C(c2ccnc(C)c2)c2ccccc2Cl)n(C)c1=O] | C75-2 Isomer 2 | Chiral SFC (CHIRALCEL OJ- H; 15% (MeOH) in Liquid CO₂ + 0.1% DEA) Chiral SFC (CHIRALCEL OX- H; 40% (IPA:MEOH; 50:50) in Hexanes + 0.1% DEA) Flow rate: 18 ml/min | 4.53 min |
| 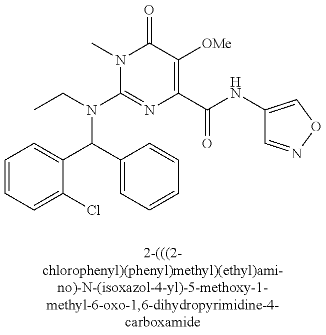 [SMILES: CCN(c1nc(C(=O)Nc2cnoc2)c(OC)c(=O)n1C)C(c1ccccc1)c1ccccc1Cl] | C76-2 Isomer 1 | Chiral SFC (CHIRALPAK IB- N; 15% (MeOH) in Liquid CO₂ + 0.1% DEA) Flow rate: 80 ml/min | 4.94 min |
| 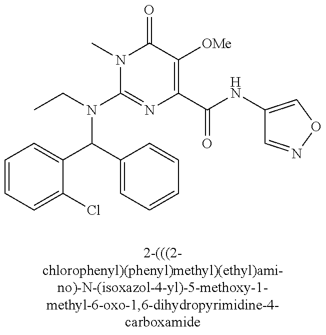 [SMILES: CCN(c1nc(C(=O)Nc2cnoc2)c(OC)c(=O)n1C)C(c1ccccc1)c1ccccc1Cl] | C76-2 Isomer 2 | Chiral SFC (CHIRALPAK IB- N; 15% (MeOH) in Liquid CO₂ + 0.1% DEA) Flow rate: 80 ml/min | 5.18 min |
| 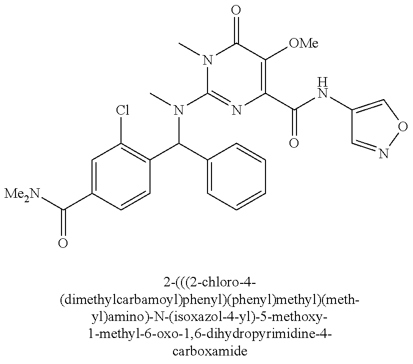 [SMILES: COc1c(C(=O)Nc2cnoc2)nc(N(C)C(c2ccccc2)c2ccc(C(=O)N(C)C)cc2Cl)n(C)c1=O] | C77-2 Isomer 1 | Chiral SFC (CHIRALPAK IC; 35% (IPA:ACN; 70:30) in Hexanes + 0.1% DEA) Flow rate: 20 ml/min | 11.57 min |
| 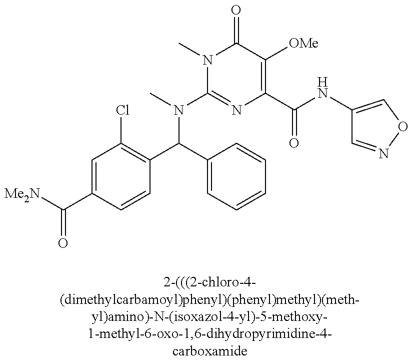 [SMILES: COc1c(C(=O)Nc2cnoc2)nc(N(C)C(c2ccccc2)c2ccc(C(=O)N(C)C)cc2Cl)n(C)c1=O] | C77-2 Isomer 2 | Chiral SFC (CHIRALPAK IC; 35% (IPA:ACN; 70:30) in Hexanes + 0.1% DEA) Flow rate: 20 ml/min | 13.00 min |
| 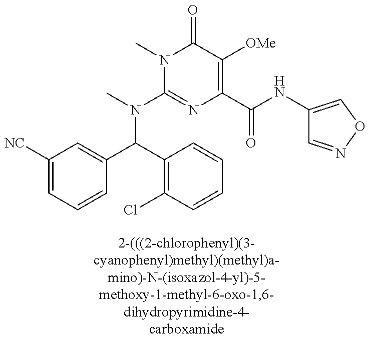 [SMILES: COc1c(C(=O)Nc2cnoc2)nc(N(C)C(c2cccc(C#N)c2)c2ccccc2Cl)n(C)c1=O] | C78-2 Isomer 1 | Chiral SFC (CHIRALCEL IC; 35% (IPA:ACN; 70:30) in Hexanes + 0.1% DEA) Flow rate: 18 ml/min | 10.57 min |
| 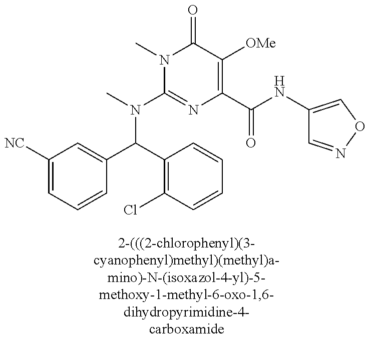 [SMILES: COc1c(C(=O)Nc2cnoc2)nc(N(C)C(c2cccc(C#N)c2)c2ccccc2Cl)n(C)c1=O] | C78-2 Isomer 2 | Chiral SFC (CHIRALCEL IC; 35% (IPA:ACN; 70:30) in Hexanes + 0.1% DEA) Flow rate: 18 ml/min | 12.54 min |
| 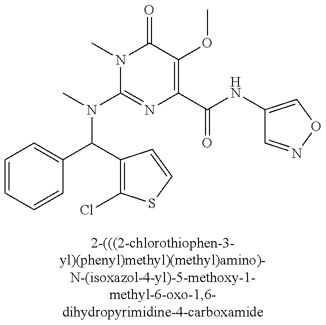 [SMILES: COc1c(C(=O)Nc2cnoc2)nc(N(C)C(c2ccccc2)c2ccsc2Cl)n(C)c1=O] | C79-2 Isomer 1 | Chiral SFC (CHIRALPAK IA- 3, 4.6*50 mm, 3 um; Mobile Phase; A: Hex:DCM (5:1)(0.1% DEA):IPA = 90:10) Flow rate: 1 ml/min | 2.51 min |
| 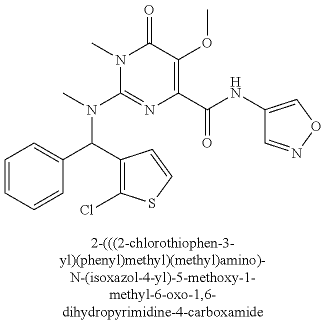 [SMILES: COc1c(C(=O)Nc2cnoc2)nc(N(C)C(c2ccccc2)c2ccsc2Cl)n(C)c1=O] | C79-2 Isomer 2 | Chiral SFC (CHIRALPAK IA- 3, 4.6*50 mm, 3 um; Mobile Phase; A: Hex:DCM (5:1)(0.1% DEA):IPA = 90:10) Flow rate: 1 ml/min | 3.23 min |

### Synthesis of 2-(dibenzylamino)-5-ethoxy-N-(isoxazol-4-yl)-1-methyl-6-oxo-1,6-dihydropyrimidine-4-carboxamide, C₁₇

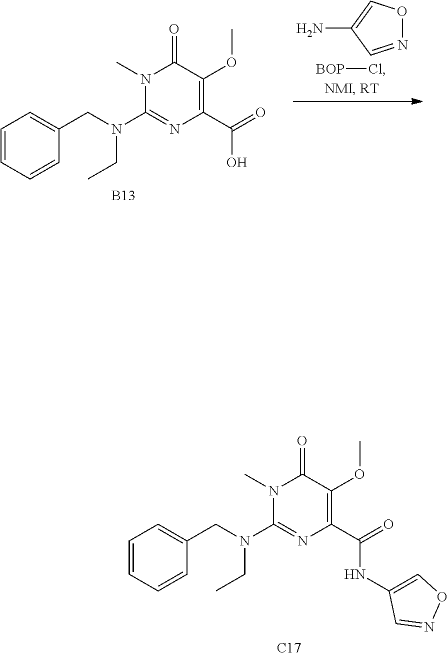

To a stirred mixture of and B13 (2-[benzyl(ethyl)amino]-5-methoxy-1-methyl-6-oxopyrimidine-4-carboxylic acid) (1.00 g, 3.15 mmol, 1.00 equiv), 1,2-oxazol-4-amine (0.530 g, 6.30 mmol, 2.00 equiv) in N,N-dimethylformamide was added 1-methyl-1H-imidazole (0.780 g, 9.45 mmol, 3.00 equiv) in portions under argon atmosphere. The resulting mixture was stirred for 0.5 min at room temperature under argon atmosphere. To the above mixture was added Bis(2-oxo-3-oxazolidinyl) phosphinic chloride (2.26 g, 4.73 mmol, 1.50 equiv) in portions over 0.5 min at 0° C. The resulting mixture was stirred for additional 2 h at room temperature and filtered, the filter cake was washed with acetonitrile. The filtrate was concentrated under reduced pressure. The residue was purified by silica gel column chromatography, eluted with petroleum ether/ethyl acetate (100:1-1:1) to afford 600 mg of C₁₇ (2-[benzyl(ethyl)amino]-5-methoxy-1-methyl-N-(1,2-oxazol-4-yl)-6-oxopyrimidine-4-carboxamide) (yield, 50%) as a yellow solid. ESI−MS m/z=384.4 [M+H]+. Calculated MW: 383.2.

### Synthesis of N-(isoxazol-4-yl)-5-methoxy-1-methyl-2-(methyl(naphthalen-1-ylmethyl)amino)-6-oxo-1,6-dihydropyrimidine-4-carboxamide, C18

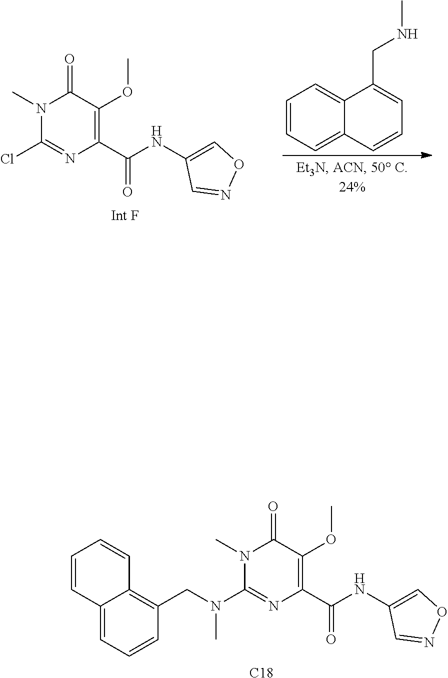

Into a 40-mL sealed tube, was placed (2-chloro-5-methoxy-1-methyl-N-(1,2-oxazol-4-yl)-6-oxopyrimidine-4-carboxamide) Int F (300 mg, 1.05 mmol, 1.00 equiv), methyl(naphthalen-1-ylmethyl) amine (217 mg, 1.26 mmol, 1.20 equiv), acetonitrile (6.00 mL), triethylamine (0.440 mL, 4.34 mmol, 3.00 equiv). The resulting solution was stirred for 2 h at 50° C. The residue was applied onto a reversed-phase column with Acetonitrile/Water (0.1% Formic Acid) (1:1) as the eluent. This resulted in 110 mg of 5-methoxy-1-methyl-2-[methyl(naphthalen-1-ylmethyl)amino]-N-(1,2-oxazol-4-yl)-6-oxopyrimidine-4-carboxamide, C18 (yield, 24%) as an orange solid. Calculated MW: 419.2 ESI−MS m/z=420.2 [M+H]⁺.

The following intermediate(s) were synthesized using similar conditions as those described in the step above along with appropriate starting materials

| | Inter- | |
|---|---|---|
| | medi- | |
| Structure | ate | LCMS |
| 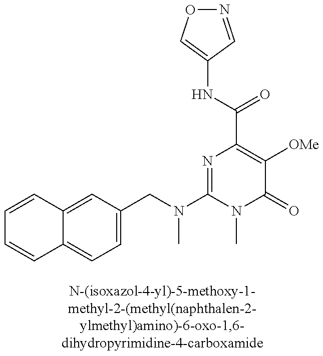 [SMILES: COc1c(C(=O)Nc2cnoc2)nc(N(C)Cc2ccc3ccccc3c2)n(C)c1=O] | C19 | ESI-MS m/Z = 420.4 [M + H]+. Calculated MW: 419.1 |
| 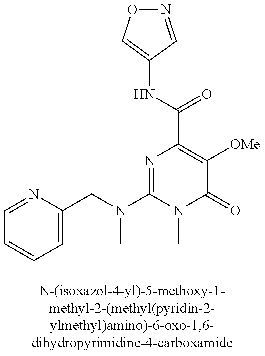 [SMILES: COc1c(C(=O)Nc2cnoc2)nc(N(C)Cc2ccccn2)n(C)c1=O] | C21 | ESI-MS m/Z = 371.2 [M + H]+. Calculated MW: 370.1 |

### Synthesis of (2-chloro-5-methoxy-1-methyl-N-(1,2-oxazol-4-yl)-6-oxopyrimidine-4-carboxamide C₂₀

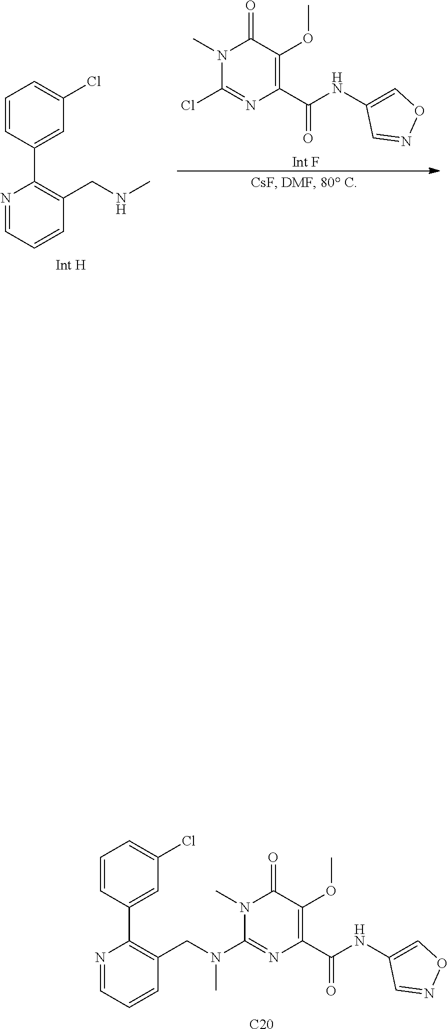

Into a 40-mL vial, was (2-chloro-5-methoxy-1-methyl-N-(1,2-oxazol-4-yl)-6-oxopyrimidine-4-carboxamide), Int H (420 mg, 1.48 mmol, 1.00 equiv), ([[2-(3-chlorophen yl)pyridin-3-yl]methyl](methyl)amine), C20 (687 mg, 2.95 mmol, 2.00 equiv), caesium fluoride (672 mg, 4.43 mmol, 3.00 equiv), N,N-dimethyl formamide (15.0 mL). The resulting solution was stirred for 12 h at 80° C. in an oil bath. The solids were filtered out. The crude product was purified by preparatory HPLC with the following conditions (IntelFlash-₁): Column, C18 silica gel; mobile phase, CH₃CN/H₂O/formic acid=10/90 increasing to CH₃CN/H₂O/formic acid=50/50; detector, 254 nm. This resulted in 300 mg of (2-([[2-(3-chlorophenyl)pyridin-3-yl]methyl](methyl)amino)-5-methoxy-1-methyl-N-(1,2-oxazol-4-yl)-6-oxopyrimidine-4-carboxamide) C20 (yield 42%) as a yellow semi-solid. ESI−MS m/z=481.2 [M+H]±. Calculated MW: 480.1

The following intermediate(s) were synthesized using similar conditions as those described in the step above along with appropriate starting materials

| Structure | Intermediate | LCMS |
|---|---|---|
| 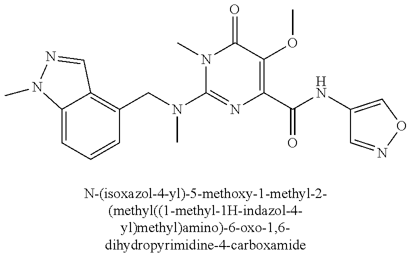 [SMILES: COc1c(C(=O)Nc2cnoc2)nc(N(C)Cc2cccc3c2cnn3C)n(C)c1=O] | C31 | ESI-MS m/z = 424.2 [M + H]+. Calculated MW: 423.2 |
| 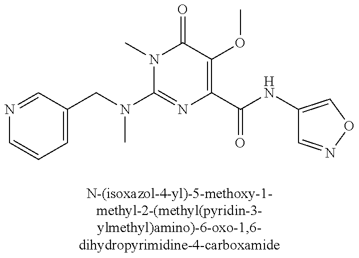 [SMILES: COc1c(C(=O)Nc2cnoc2)nc(N(C)Cc2cccnc2)n(C)c1=O] | C32 | ESI-MS m/z = 371.2 [M + H]⁺ Calculated MW: 370.1 |
| 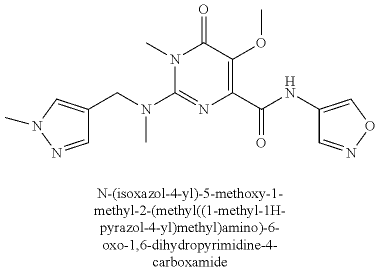 [SMILES: COc1c(C(=O)Nc2cnoc2)nc(N(C)Cc2cnn(C)c2)n(C)c1=O] | C33 | ESI-MS m/z = 374.1 [M + H]+. Calculated MW = 373.2 |
| 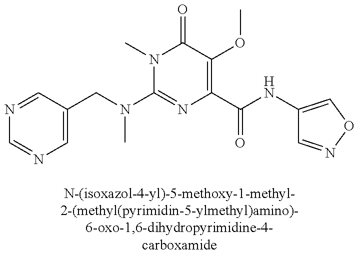 [SMILES: COc1c(C(=O)Nc2cnoc2)nc(N(C)Cc2cncnc2)n(C)c1=O] | C34 | ESI-MS m/z = 372.2 [M + H]+. Calculated MW: 371.1 |
| 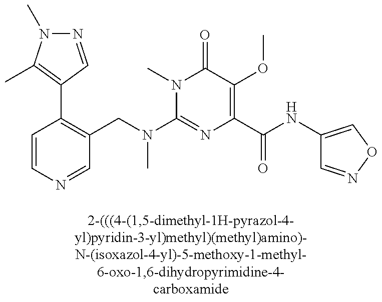 [SMILES: COc1c(C(=O)Nc2cnoc2)nc(N(C)Cc2cnccc2-c2cnn(C)c2C)n(C)c1=O] | C35 | ESI-MS m/Z = 465.2 [M + H]+. Calculated MW: 464.2 |
| 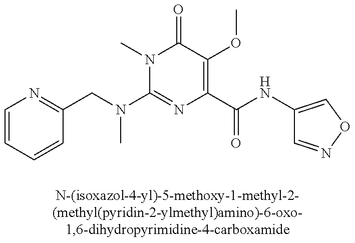 [SMILES: COc1c(C(=O)Nc2cnoc2)nc(N(C)Cc2ccccn2)n(C)c1=O] | C36 | ESI-MS m/z = 371.2 [M + H]+. Caclulated MW: 370.1 |
| 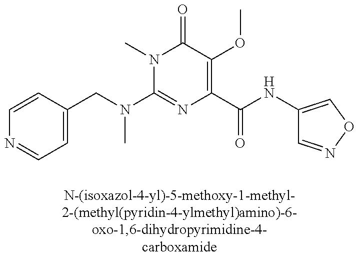 [SMILES: COc1c(C(=O)Nc2cnoc2)nc(N(C)Cc2ccncc2)n(C)c1=O] | C37 | ESI-MS m/z = 371.3 [M + H]+. Calculated MW: 370.1 |
| 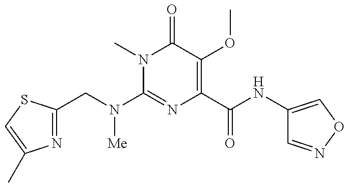 [SMILES: COc1c(C(=O)Nc2cnoc2)nc(N(C)Cc2nc(C)cs2)n(C)c1=O] | C38 | ESI-MS m/z = 413.1 [M + Na]+. Calculated MW: 390.1 |

### Synthesis of 2-((1-acetylpiperidin-4-yl)(methyl)amino)-5-ethoxy-N-(isoxazol-4-yl)-1-methyl-6-oxo-1,6-dihydropyrimidine-4-carboxamide, C64-2

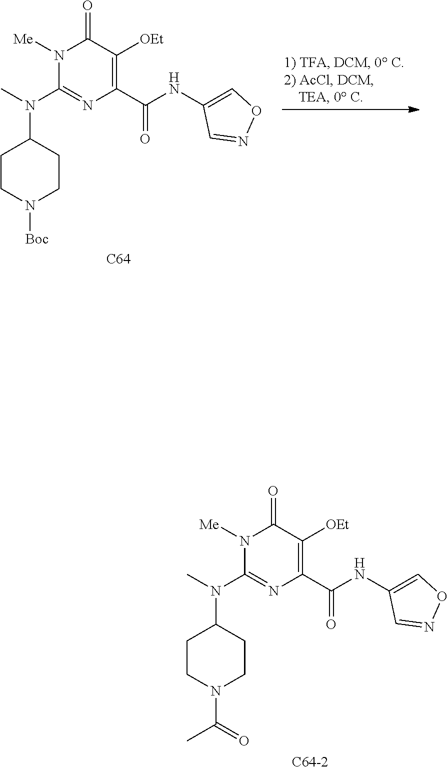 [SMILES: CCOc1c(C(=O)Nc2cnoc2)nc(N(C)C2CCN(C(=O)OC(C)(C)C)CC2)n(C)c1=O ; "C64" ; CCOc1c(C(=O)Nc2cnoc2)nc(N(C)C2CCN(C(C)=O)CC2)n(C)c1=O ; "C64-2"]

To an ice cold solution of tert-Butyl 4-((5-ethoxy-4-(isoxazol-4-ylcarbamoyl)-1-methyl-6-oxo-1,6-dihydropyrimidin-2-yl) (methyl)amino) piperidine-1-carboxylate (0.2 g, 0.42 mmol) in dichloromethane (2 mL), TFA (0.6 mL) was added dropwise under nitrogen atmosphere at 0° C. The reaction mixture was stirred for 2 h at room temperature. After completion of reaction (monitored by TLC), the reaction mixture was concentrated to get crude compound. The crude compound was triturated with n-hexanes (4×1 mL) and obtained solid was dried under vacuum to get pure title compound (0.216 g). LCMS: ESI−MS m/z=377.6 [M+H]+. Calculated MW: 476.42

To an ice cold solution of TFA salt of 5-Ethoxy-N-(isoxazol-4-yl)-1-methyl-2-(methyl(piperidin-4-yl) amino)-6-oxo-1,6-dihydropyrimidine-4-carboxamide (0.22 g, 0.44 mmol) in dichloromethane (10.8 mL), triethylamine (0.112 g, 1.10 mmol) was added under nitrogen atmosphere at 0° C. To the above reaction mixture, acetyl chloride (0.038 g, 0.48 mmol) was added dropwise at 0° C. The reaction mixture was further stirred for 2 h at 0° C. After completion of reaction (monitored by TLC), the reaction mixture was concentrated to get crude product. The crude product was purified by column chromatography using n-hexanes:ethyl acetate to get pure title compound (0.15 g, 87%) (2 steps).

LCMS: ESI−MS m/z=417.6 [M−H]+. Calculated MW: 418.45

### Synthesis of 2-(dibenzylamino)-5-ethoxy-N-(isoxazol-4-yl)-1-methyl-6-oxo-1,6-dihydropyrimidine-4-carboxamide, Example 1

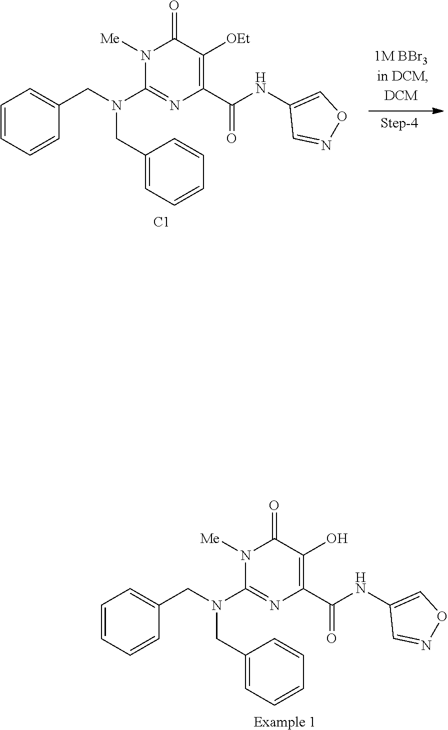

To a stirred solution of 2-(dibenzylamino)-5-ethoxy-1-methyl-N-(1,2-oxazol-4-yl)-6-oxo-1,6-dihydropyrimidine-4-carboxamide, C1 (91 mg, 0.20 mmol) in dichloromethane (1.32 mL) was added boron tribromide (990 μL, 990 μmol) dropwise at −60° C. under argon atmosphere. The resulting mixture was stirred for 40 min at −30° C. and then quenched with 1.5 mL of methanol at −30° C. The resulting mixture was diluted with 2 mL of toluene. The solvent was removed under reduced pressure. The resulting residue was purified by reverse phase chromatography to give 11.8 mg of 2-(dibenzylamino)-5-ethoxy-N-(isoxazol-4-yl)-1-methyl-6-oxo-1,6-dihydropyrimidine-4-carboxamide, Example 1 in 13% yield. Calculated MW: 431.4 ESI−MS m/z=432.1 [M+H]⁺. 1H NMR (400 MHz,DMSO-d6) δ=11.30-11.02 (m, 1H), 10.52-10.27 (m, 1H), 9.29 (s, 1H), 8.91 (s, 1H), 7.46-7.16 (m, 10H), 4.36 (s, 4H), 3.58 (s, 3H)

| Structure | Ex. | LCMS | ¹HNMR |
|---|---|---|---|
| 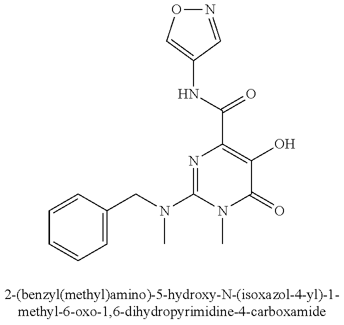 [SMILES: CN(Cc1ccccc1)c1nc(C(=O)Nc2cnoc2)c(O)c(=O)n1C] | 2 | ESI-MS m/z = 356.3 [M + H]+. Calculated MW: 355.3 | 1H NMR (400 MHz, DMSO- d6) δ 11.19 (s, 1H), 10.51 (s, 1H), 9.31 (s, 1H), 8.92 (s, 1H), 7.29-7.39 (m, 5H), 4.40 (s, 2H), 3.49 (s, 3H), 2.74 (s, 3H). |
| 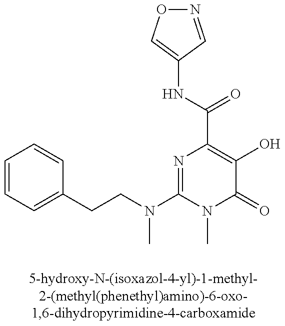 [SMILES: CN(CCc1ccccc1)c1nc(C(=O)Nc2cnoc2)c(O)c(=O)n1C] | 3 | ESI-MS m/z = 370.3 [M + H]+. Calculated MW: 369.3 | 1H NMR (300 MHz, DMSO- d6) δ 11.15 (s, 1H), 10.49 (s, 1H), 9.32 (s, 1H), 8.92 (s, 1H), 7.19-7.26 (m, 5H), 3.43 (t, J = 7.2 Hz, 2H), 3.28 (s, 3H), 2.88-2.90 (m, 2H), 2.87 (s, 3H). |
| 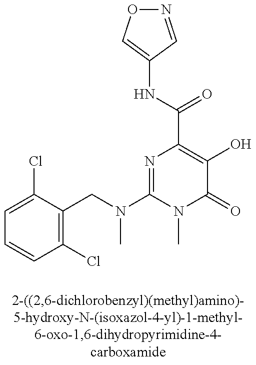 [SMILES: CN(Cc1c(Cl)cccc1Cl)c1nc(C(=O)Nc2cnoc2)c(O)c(=O)n1C] | 4 | ESI-MS m/z = 424.2 [M + H]+. Calculated MW: 423.1 | 1H NMR (400 MHz, DMSO- d6): δ 11.05 (s, 1H), 10.37 (s, 1H), 9.25 (s, 1H), 8.98 (s, 1H), 7.53 (d, J = 8 Hz, 2H), 7.41 (t, J = 8.4, 1H), 4.78 (s, 2H), 3.44 (s, 3H), 2.73 (s, 3H). |
| 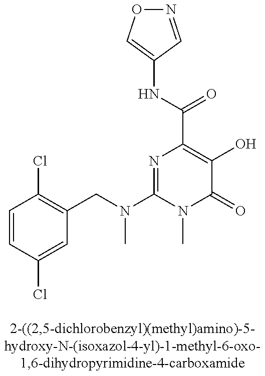 [SMILES: CN(Cc1cc(Cl)ccc1Cl)c1nc(C(=O)Nc2cnoc2)c(O)c(=O)n1C] | 5 | ESI-MS m/z = 424.2 [M + H]+. calculated MW: 423.1 | 1H NMR (400 MHz, DMSO- d6): δ 11.20 (s, 1H), 10.57 (s, 1H), 9.33 (s, 1H), 8.92 (s, 1H), 7.74 (s, 1H), 7.52 (d, J = 8.4 Hz, 1H), 7.41-7.44 (dd, J = 8.4 Hz, 1H), 4.52 (s, 2H), 3.42 (s, 3H), 2.79 (s, 3H). |
| 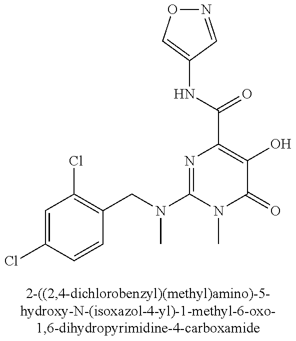 [SMILES: CN(Cc1ccc(Cl)cc1Cl)c1nc(C(=O)Nc2cnoc2)c(O)c(=O)n1C] | 6 | ESI-MS m/z = 424.2 [M + H]+. Calculated MW: 423.1 | 1H NMR (400 MHz, DMSO- d6): δ 11.20 (s, 1H), 10.54 (s, 1H), 9.33 (s, 1H), 8.92 (s, 1H), 7.61-7.66 (m, 2H), 7.45- 7.48 (dd, J = 8 Hz, 1H), 4.51 (s, 2H), 3.42 (s, 3H), 2.76 (s, 3H). |
| 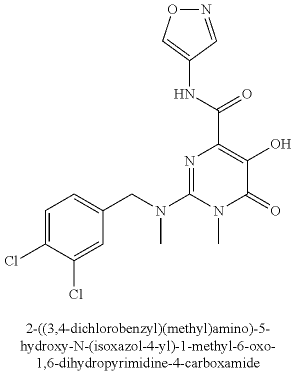 [SMILES: CN(Cc1ccc(Cl)c(Cl)c1)c1nc(C(=O)Nc2cnoc2)c(O)c(=O)n1C] | 7 | ESI-MS m/z = 424.2 [M + H]+. Calculated MW: 423.1 | 1H NMR (400 MHz, DMSO- d6): δ 11.26 (s, 1H), 10.57 (s, 1H), 9.31 (s, 1H), 8.90 (s, 1H), 7.70 (s, 1H), 7.61 (d, J = 8 Hz, 1H), 7.39 (d, J = 8 Hz, 1H), 4.40 (s, 2H), 3.45 (s, 3H), 2.73 (s, 3H). |
| 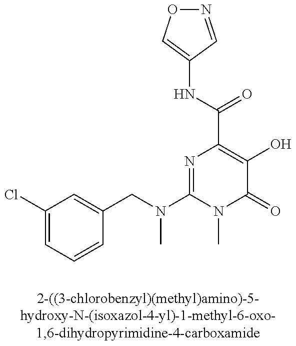 [SMILES: CN(Cc1cccc(Cl)c1)c1nc(C(=O)Nc2cnoc2)c(O)c(=O)n1C] | 8 | ESI-MS m/z = 389.8 [M + H]+. Calculated MW: 389.1 | 1H NMR (400 MHz, DMSO- d6): δ 11.18 (s, 1H), 10.50 (s, 1H), 9.32 (s, 1H), 8.92 (s, 1H), 7.50 (s, 1H), 7.36-7.41 (m, 3H), 4.43 (s, 2H), 3.48 (s, 3H), 2.76 (s, 3H). |
| 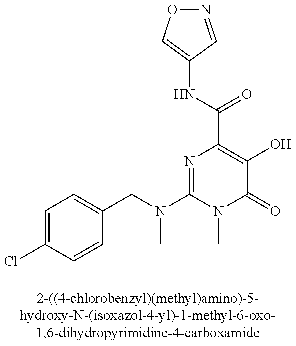 [SMILES: CN(Cc1ccc(Cl)cc1)c1nc(C(=O)Nc2cnoc2)c(O)c(=O)n1C] | 9 | ESI-MS m/z = 389.8 [M + H]+. Calculated MW: 389.1 | 1H NMR (400 MHz, DMSO- d6): δ 11.18 (s, 1H), 10.45 (s, 1H), 9.33 (s, 1H), 8.93 (s, 1H), 7.46 (d, J = 20 Hz, 4H), 4.42 (s, 2H), 3.48 (s, 3H), 2.74 (s, 3H). |
| 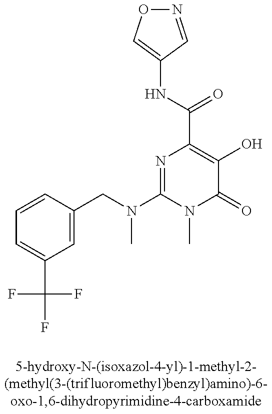 [SMILES: CN(Cc1cccc(C(F)(F)F)c1)c1nc(C(=O)Nc2cnoc2)c(O)c(=O)n1C] | 10 | ESI-MS m/z = 424.4 [M + H]+. Calculated MW: 423.4 | 1H NMR (400 MHz, DMSO- d6): δ 11.18 (s, 1H), 10.52 (s, 1H), 9.31 (s, 1H), 8.89 (s, 1H), 7.51-7.80 (m, 4H), 4.58 (s, 2H), 3.40 (s, 3H), 2.74 (s, 3H). |
| 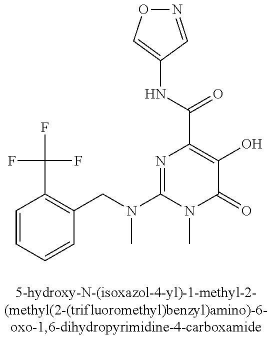 [SMILES: CN(Cc1ccccc1C(F)(F)F)c1nc(C(=O)Nc2cnoc2)c(O)c(=O)n1C] | 11 | ESI-MS m/z = 424.5 [M + H]+. Calculated MW: 423.4 | 1H NMR (400 MHz, DMSO- d6): δ 11.7 (s, 1H), 10.47 (s, 1H), 9.32 (s, 1H), 8.92 (s, 1H), 7.80 (s, 1H), 7.58-7.73 (m, 3H), 4.54 (s, 2H), 3.49 (s, 3H), 2.79 (s, 3H). |
| 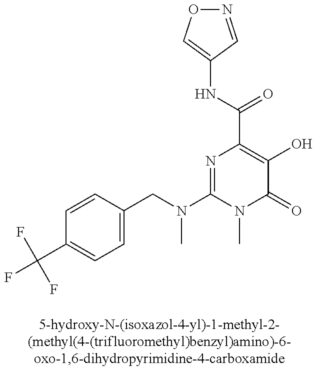 [SMILES: CN(Cc1ccc(C(F)(F)F)cc1)c1nc(C(=O)Nc2cnoc2)c(O)c(=O)n1C] | 12 | ESI-MS m/z = 424.4 [M + H]+. Calculated MW: 423.4 | 1H NMR (400 MHz, DMSO- d6): δ 11.19 (s, 1H), 10.54 (s, 1H), 9.32 (s, 1H), 8.92 (s, 1H), 7.75 (d, J = 8 Hz, 2H), 7.65 (d, J = 8 Hz, 2H), 4.53 (s, 2H), 3.49 (s, 3H), 2.77 (s, 3H). |
| 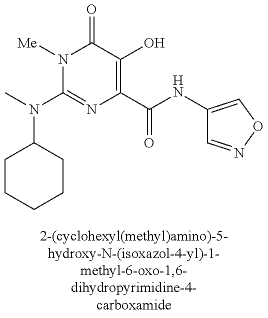 [SMILES: CN(c1nc(C(=O)Nc2cnoc2)c(O)c(=O)n1C)C1CCCCC1] | 72 | ESI-MS m/z = 348.3 [M + H]+. Calculated MW: 347.4 | ¹H NMR (400 MHz, DMSO- d₆): δ 1.01-1.02 (m, 2H), 1.28- 1.38 (m, 2H), 1.46-1.54 (m, 2H), 1.55-1.65 (m, 4H), 2.73 (s, 3H), 3.11 (t, J = 10.8 Hz, 1H), 3.40 (s, 3H), 8.91 (s, 1H), 9.30 (s, 1H), 10.38 (bs, 1H), 11.17 (bs, 1H). |
| 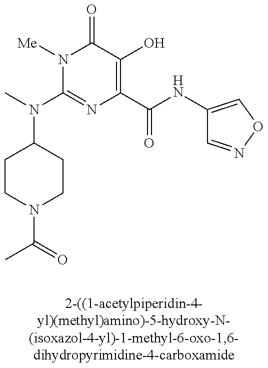 [SMILES: CC(=O)N1CCC(N(C)c2nc(C(=O)Nc3cnoc3)c(O)c(=O)n2C)CC1] | 73 | ESI-MS m/z = 391.5 [M + H]+. Calculated MW: 390.4 | ¹H NMR (400 MHz, DMSO- d₆): δ 1.52-1.71 (m, 4H), 2.00 (s, 3H), 2.50- 2.67 (m, 2H), 2.71 (s, 3H), 3.06-3.12 (m, 1H), 3.43 (s, 3H), 3.80-3.84 (m, 1H), 4.37- 4.41 (m, 1H), 8.91 (s, 1H), 9.31 (s, 1H), 10.41 (bs, 1H), 11.8 (bs, 1H). |
| 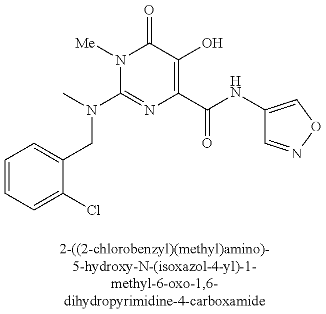 [SMILES: CN(Cc1ccccc1Cl)c1nc(C(=O)Nc2cnoc2)c(O)c(=O)n1C] | 74 | ESI-MS m/z = 390.2 [M + H]+. Calculated MW: 389.8 | ¹H NMR (400 MHz, DMSO- d₆): δ 2.76 (s, 3H), 3.42 (s, 3H), 4.52 (s, 2H), 7.35-7.38 (m, 2H), 7.49 (d, J = 7.6 Hz, 1H), 7.59 (d, J = 6.8 Hz, 1H), 8.92 (s, 1H), 9.33 (s, 1H), 10.53 (bs, 1H), 11.195 (bs, 1H). |
| 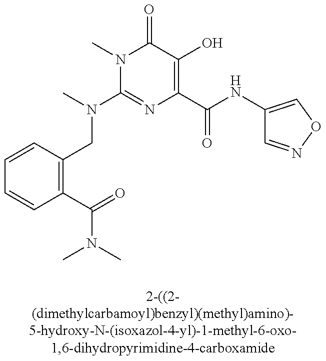 [SMILES: CN(C)C(=O)c1ccccc1CN(C)c1nc(C(=O)Nc2cnoc2)c(O)c(=O)n1C] | 75 | ESI-MS m/z = 427.6 [M + H]+. Calculated MW: 426.4 | ¹H NMR (400 MHz, DMSO- d₆): δ 2.80 (s, 3H), 2.85 (s, 3H), 3.10 (s, 3H), 3.53 (s, 3H), 4.37 (bs, 2H), 7.27 (d, J = 6.8, 1H), 7.33-7.36 (t, J = 7.6, 2H), 7.47 (d, J = 7.2, 1H), 8.95 (s, 1H), 9.30 (s, 1H), 10.95 (s, 1H), 11.31 (s, 1H). |
| 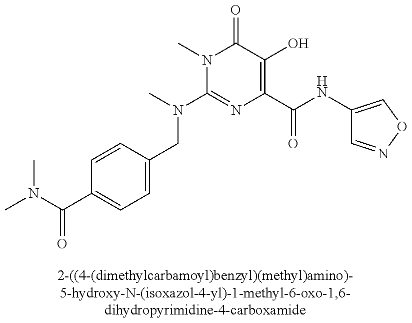 [SMILES: CN(C)C(=O)c1ccc(CN(C)c2nc(C(=O)Nc3cnoc3)c(O)c(=O)n2C)cc1] | 76 | ESI-MS m/z = 427.3 [M + H]+. Calculated MW: 426.4 | ¹H NMR (400 MHz, DMSO- d₆): δ 2.67 (s, 3H), 2.91 (s, 3H), 2.98 (s, 3H), 3.48 (s, 3H), 4.32 (bs, 2H), 7.39 (d, J = 7.6 Hz, 2H), 7.45 (d, J = 7.6 Hz, 2H), 8.86 (s, 1H), 9.25 (s, 1H), 11.72 (s, 1H), 11.86 (s, 1H). |
| 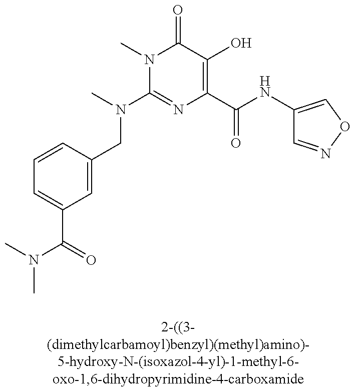 [SMILES: CN(C)C(=O)c1cccc(CN(C)c2nc(C(=O)Nc3cnoc3)c(O)c(=O)n2C)c1] | 77 | ESI-MS m/z = 427.3 [M + H]+. Calculated MW: 426.4 | ¹H NMR (400 MHz, DMSO- d₆): δ 2.75 (s, 3H), 2.84 (s, 3H), 2.94 (s, 3H), 3.49 (s, 3H), 4.43 (s, 2H), 7.29 (d, J = 6.8 Hz, 1H), 7.42-7.45 (m, 3H), 8.90 (s, 1H), 9.30 (s, 1H), 10.52 (bs, 1H), 11.17 (bs, 1H). |
| 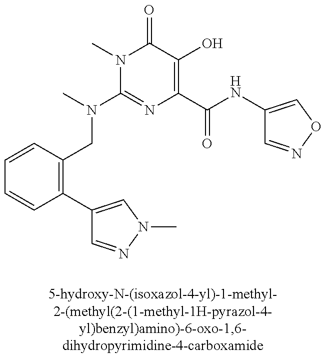 [SMILES: CN(Cc1ccccc1-c1cnn(C)c1)c1nc(C(=O)Nc2cnoc2)c(O)c(=O)n1C] | 78 | ESI-MS m/z = 436.3 [M + H]+. Calculated MW: 435.4 | ¹H NMR (400 MHz, DMSO- d₆): δ 2.67 (s, 3H), 3.27 (s, 3H), 3.81 (s, 3H), 4.50 (s, 2H), 7.33-7.34 (m, 3H), 7.56- 7.59 (m, 2H), 7.88 (s, 1H), 8.91 (s, 1H), 9.33 (s, 1H), 10.44 (bs, 1H), 11.13 (bs, 1H). |
| 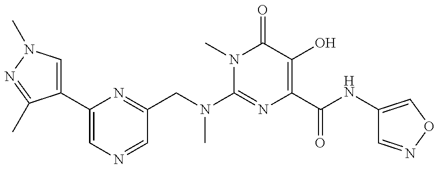 [SMILES: Cc1nn(C)cc1-c1cncc(CN(C)c2nc(C(=O)Nc3cnoc3)c(O)c(=O)n2C)n1] | 79 | ESI-MS m/z = 452.6 [M + H]+. Calculated MW: 451.5 | ¹H NMR (400 MHz, DMSO- d₆): δ 2.36 (s, 3H), 2.85 (s, 3H), 3.54 (s, 3H), 3.79 (s, 3H), 4.55 (s, 2H), 8.29 (s, 1H), 8.48 (s, 1H), 8.77 (s, 1H), 8.87 (s, |
| | | | 1H), 9.29 (s, |
| | | | 1H), 10.50 (s, |
| | | | 1H), 11.16 (s, |
| | | | 1H). |
| 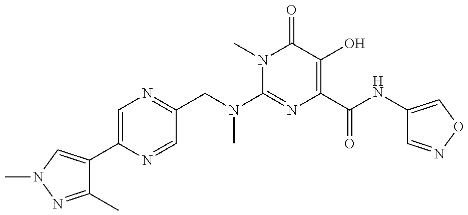 [SMILES: Cc1nn(C)cc1-c1cnc(CN(C)c2nc(C(=O)Nc3cnoc3)c(O)c(=O)n2C)cn1] | 80 | ESI-MS m/z = 452.7 [M + H]+. Calculated MW: 451.5 | ¹H NMR (400 MHz, DMSO- d₆): δ 2.42 (s, 3H), 2.81 (s, 3H), 3.52 (s, 3H), 3.80 (s, 3H), 4.52 (s, 2H), 8.32 (s, 1H), 8.67 (s, 1H), 8.82 (s, 1H), 8.89 (s, 1H), 9.30 (s, 1H), 10.51 (s, 1H), 11.20 (s, |
| | | | 1H). |

### Synthesis of 2-(benzyl(ethyl)amino)-5-hydroxy-N-(isoxazol-4-yl)-1-methyl-6-oxo-1,6-dihydropyrimidine-4-carboxamide, Example 13

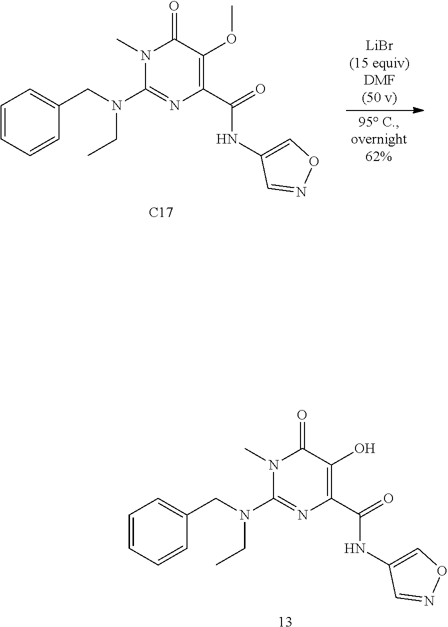 [SMILES: CCN(Cc1ccccc1)c1nc(C(=O)Nc2cnoc2)c(OC)c(=O)n1C ; "C17" ; CCN(Cc1ccccc1)c1nc(C(=O)Nc2cnoc2)c(O)c(=O)n1C ; "13"]

Into a 40 mL vial were added 2-[benzyl(ethyl)amino]-5-methoxy-1-methyl-N-(1,2-oxazol-4-yl)-6-oxopyrimidine-4-carboxamide, C₁₇ (100 mg, 0.261 mmol, 1.00 equiv) and lithium bromide (340 mg, 3.91 mmol, 15.0 equiv) in N,N-dimethylformamide (5.00 mL) at room temperature. The resulting mixture was stirred for overnight at 95° C. under argon atmosphere. The crude product was purified by preparatory HPLC with the following conditions (Column: XSelect CSH Prep C18 OBD Column, 5 um, 19*150 mm; Mobile Phase A:Water(0.05% trifluoro acetic acid), Mobile Phase B: acetonitrile; Flow rate: 25 mL/min; Gradient:40 B to 55 B in 8 min; 254/220 nm) to afford 60 mg of 2-[benzyl(ethyl)amino]-5-hydroxy-1-methyl-N-(1,2-oxazol-4-yl)-6-oxopyrimidine-4-carboxamide, Example 13 (yield, 62%) as a white solid. ESI−MS m/z=370.1 [M+H]+. Calculated MW: 369.1. 1H NMR (400 MHz, DMSO-d6) δ 11.19 (s, 1H), 10.46 (s, 1H), 9.31 (s, 1H), 8.94 (s, 1H), 7.40-7.38 (m, 2H), 7.33-7.31 (m, 2H), 7.29-7.20 (m, 1H), 4.46 (s, 2H), 3.48 (s, 3H), 3.17 (q, J=7.0 Hz, 2H), 1.13 (t, J=7.0 Hz, 3H).

The following examples were synthesized using similar conditions as those described in the step above along with appropriate starting materials.

| Structure | Ex. | LCMS | ¹H NMR |
|---|---|---|---|
| 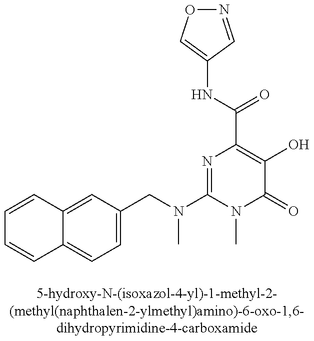 [SMILES: CN(Cc1ccc2ccccc2c1)c1nc(C(=O)Nc2cnoc2)c(O)c(=O)n1C] | 14 | ESI-MS m/z = 406.1 [M + H]+. Calculated MW: 405.1 | 1H NMR (400 MHz, DMSO- d6) δ 11.19 (s, 1H), 10.62 (s, 1H), 9.31 (s, 1H), 8.93 (s, 1H), 7.94-7.90 (m, 4H), 7.52 (ddd, J = 9.3, 6.7, 1.9 Hz, 3H), 4.57 (s, 2H), 3.53 (s, 3H), 2.78 (s, 3H). |
| 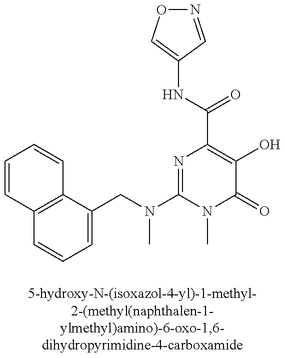 [SMILES: CN(Cc1cccc2ccccc12)c1nc(C(=O)Nc2cnoc2)c(O)c(=O)n1C] | 15 | ESI-MS m/z = 406.1 [M + H]+. Calculated MW: 405.1 | 1H NMR (300 MHz, DMSO- d6) δ 11.24 (s, 1H), 10.65 (s, 1H), 9.32 (s, 1H), 8.93 (s, 1H), 8.14-8.07 (m, 1H), 7.97- 7.88 (m, 2H), 7.67 (d, J = 6.9 Hz, 1H), 7.58- 7.49 (m, 3H), 4.88 (s, 2H), 3.35 (s, 3H), 2.74 (s, 3H). |
| 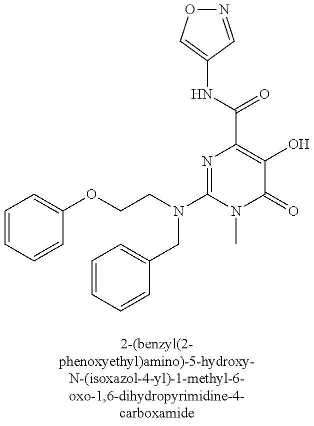 [SMILES: Cn1c(N(CCOc2ccccc2)Cc2ccccc2)nc(C(=O)Nc2cnoc2)c(O)c1=O] | 16 | ESI-MS m/z = 462.2 [M + H]+. Calculated MW: 461.7 | 1H NMR (300 MHz, DMSO- d6) δ 11.23 (s, 1H), 10.46 (s, 1H), 9.30 (s, 1H), 8.91 (s, 1H), 7.43-7.41 (m, 2H), 7.41- 7.27 (m, 2H), 7.27-7.20 (m, 3H), 6.90 (tt, J = 7.3, 1.1 Hz, 1H), 6.88-6.80 (m, 2H), 4.51 (s, 2H), 4.17 (t, J = 5.1 Hz, 2H), 3.59 (t, J = 5.1 Hz, 2H), 3.32 (s, 2H). |
| 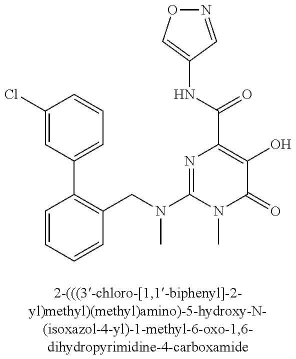 [SMILES: CN(Cc1ccccc1-c1cccc(Cl)c1)c1nc(C(=O)Nc2cnoc2)c(O)c(=O)n1C] | 17 | ESI-MS m/z = 466.1 [M + H]+. Calculated MW: 465.1 | 1H NMR (300 MHz, DMSO- d6) δ: 11.07 (s, 1H), 10.35 (s, 1H), 9.30 (s, 1H), 8.90 (s, 1H), 7.62-7.59 (m, 1H), 7.46- 7.37 (m, 3H), 7.33-7.25 (m, 4H), 4.56 (s, 2H), 3.10 (s, 3H), 2.58 (s, 3H). |
| 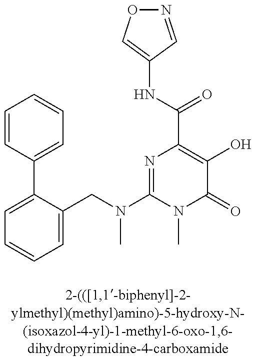 [SMILES: CN(Cc1ccccc1-c1ccccc1)c1nc(C(=O)Nc2cnoc2)c(O)c(=O)n1C] | 18 | ESI-MS m/z = 432.1 [M + H]+. Calculated MW: 431.2 | 1H NMR (300 MHz, DMSO- d6) δ: 11.05 (s, 1H), 10.50 (s, 1H), 9.30 (s, 1H), 8.90 (s, 1H), 7.61 (d, J = 3.0 Hz, 1H), 7.43-7.36 (m, 2H), 7.34-7.29 (m, 5H), 7.27- 7.22 (m, 1H), 4.49 (s, 2H), 3.10 (s, 3H), 2.55 (s, 3H). |
| 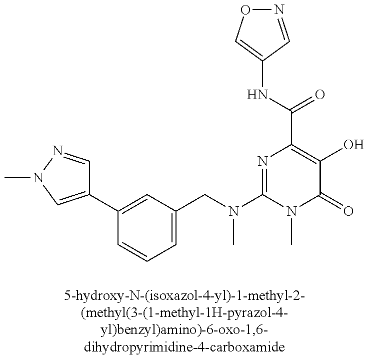 [SMILES: CN(Cc1cccc(-c2cnn(C)c2)c1)c1nc(C(=O)Nc2cnoc2)c(O)c(=O)n1C] | 19 | ESI-MS m/z = 436.2 [M + H]+. Calculated MW: 435.2 | 1H NMR (300 MHz, DMSO- d6) δ 11.16 (s, 1H), 10.46 (s, 1H), 9.31 (s, 1H), 8.92 (s, 1H), 8.10 (s, 1H), 7.83 (d, J = 0.8 Hz, 1H), 7.58 (d, J = 1.7 Hz, 1H), 7.45 (dt, J = 7.7, 1.5 Hz, 1H), 7.34 (t, J = 7.6 Hz, 1H), 7.21 (dt, J = 7.6, 1.5 Hz, 1H), 4.41 (s, 2H), 3.84 (s, 3H), 3.61 (s, 3H), 2.77 (s, 3H). |
| 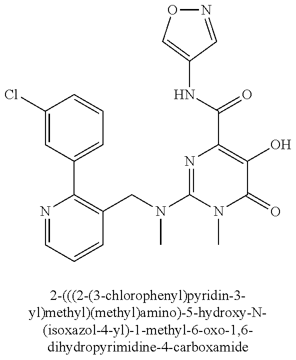 [SMILES: CN(Cc1cccnc1-c1cccc(Cl)c1)c1nc(C(=O)Nc2cnoc2)c(O)c(=O)n1C] | 20 | ESI-MS m/z = 467.1 [M + H]+. Calculated MW: 466.1 | 1H NMR (300 MHz, DMSO- d6) δ: 11.09 (s, 1H), 10.40 (s, 1H), 9.30 (s, 1H), 8.90 (s, 1H), 8.60 (d, J = 4.2 Hz, 1H), 8.03 (d, J = 8.1 Hz, 1H), 7.58 (s, 1H), 7.50- 7.36 (m, 4H), 4.63 (s, 2H), 3.12 (s, 3H), 2.58 (s, 3H). |
| 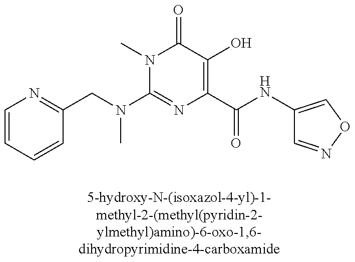 [SMILES: CN(Cc1ccccn1)c1nc(C(=O)Nc2cnoc2)c(O)c(=O)n1C] | 22 | ESI-MS m/Z = 357.1 [M + H]⁺. Calculated MW: 356.1 | 1H NMR (300 MHz, DMSO- d6) δ 11.17 (s, 1H), 10.62 (s, 1H), 9.30 (s, 1H), 8.90 (s, 1H), 7.84-7.79 (m, 1H), 7.50- 7.48 (m, 1H), 7.33-7.29 (m, 1H), 4.49 (s, 2H), 3.52 (s, 3H), 2.80 (s, 3H). |
| 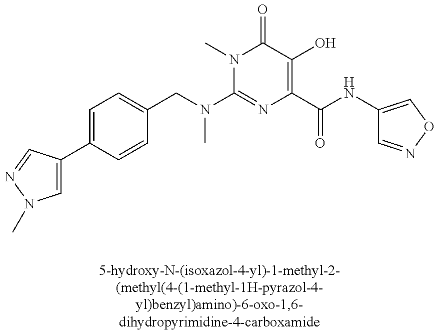 [SMILES: CN(Cc1ccc(-c2cnn(C)c2)cc1)c1nc(C(=O)Nc2cnoc2)c(O)c(=O)n1C] | 25 | ESI-MS m/Z = 436.2 [M + H]⁺. Calculated MW: 435.2 | ¹H NMR (400 MHz, DMSO- d6) δ 11.03 (s, 1H), 9.28 (s, 1H), 8.89 (s, 1H), 8.10 (s, 1H), 7.83 (s, 1H), 7.53 (d, J = 7.8 Hz, 3H), 7.35 (d, J = 7.7 Hz, 2H), 4.32 (s, 2H), 3.85 (s, 4H), 3.48 (s, 3H), 2.70 (s, 3H), 2.08 (s, 1H). |
| 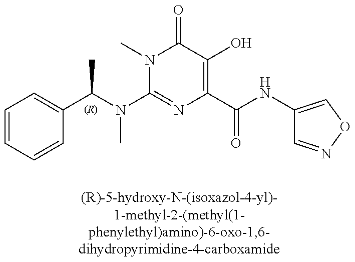 | 27 | ESI-MS m/z = 370.1 [M + H]+. Calculated MW: 369.1 | ¹H NMR (300 MHz, DMSO- d₆) δ 11.19 (s, 1H), 10.38 (s, 1H), 9.31 (s, 1H), 8.92 (s, 1H), 7.40-7.32 (m, 4H), 7.28- 7.22 (m, 1H), 4.88-4.81 (m, 1H), 3.52 (s, 3H), 2.61 (s, 3H), 1.48 (d, 3H). |
| 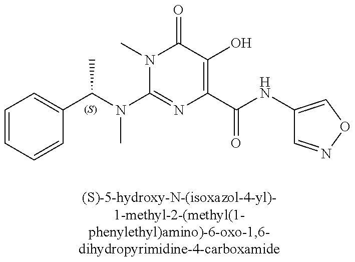 | 28 | ESI-MS m/z = 370.1 [M + H]+. Caclulated MW: 369.1 | ¹H NMR (300 MHz, DMSO- d₆) δ 11.19 (s, 1H), 10.38 (s, 1H), 9.31 (s, 1H), 8.92 (s, 1H), 7.40-7.32 (m, 4H), 7.28- 7.22 (m, 1H), 4.88-4.81 (m, 1H), 3.52 (s, 3H), 2.61 (s, 3H), 1.48 (d, J = 6.6 Hz, 3H). |
| 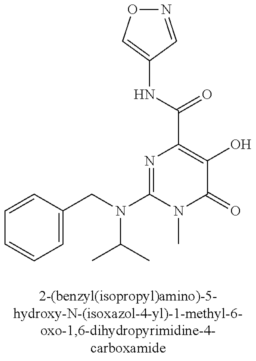 [SMILES: CC(C)N(Cc1ccccc1)c1nc(C(=O)Nc2cnoc2)c(O)c(=O)n1C] | 29 | ESI-MS m/Z = 384.2 [M + H]+. Calculated MW: 383.2 | ¹H NMR (400 MHz, DMSO- d6) δ 11.17 (s, 1H), 10.46 (s, 1H), 9.33 (s, 1H), 8.99 (s, 1H), 7.42-7.34 (m, 2H), 7.23 (t, J = 7.6 Hz, 2H), 7.17-7.10 (m, 1H), 4.49 (s, 2H), 3.60 (p, J = 6.6 Hz, 1H), 3.45 (s, 3H), 1.27 (d, J = 6.5 Hz, 6H). |
| 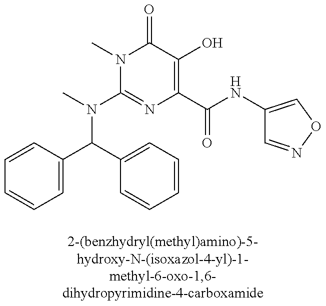 [SMILES: CN(c1nc(C(=O)Nc2cnoc2)c(O)c(=O)n1C)C(c1ccccc1)c1ccccc1] | 30 | ESI-MS m/Z = 432.2 [M + H]+. Calculated MW: 431.2 | ¹H NMR (300 MHz, DMSO- d₆) δ 11.06 (brs, 1H), 10.25 (brs, 1H), 9.30 (s, 1H), 8.97 (s, 1H), 7.60-7.44 (m, 4H), 7.28 (t, J = 7.6 Hz, 4H), 7.22-7.06 (m, 2H), 5.90 (s, 1H), 3.66 (s, 3H), 2.60 (s, 3H). |
| 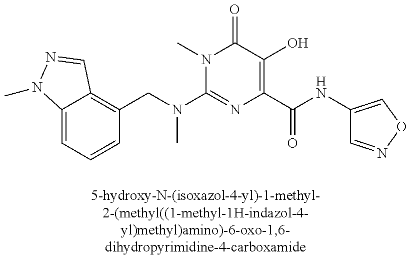 [SMILES: CN(Cc1cccc2c1cnn2C)c1nc(C(=O)Nc2cnoc2)c(O)c(=O)n1C] | 31 | ESI-MS m/Z = 410.2 [M + H]+. Calculated MW: 409.2 | 1H NMR (300 MHz, DMSO- d6) δ 11.23 (s, 1H), 10.46 (s, 1H), 9.32 (s, 1H), 8.92 (s, 1H), 8.16 (d, J = 1.0 Hz, 1H), 7.58 (d, J = 8.4 Hz, 1H), 7.38 (dd, J = 8.5, 6.9 Hz, 1H), 7.22 (d, J = 6.9 Hz, 1H), 4.73 (s, 2H), 4.04 (s, 3H), 3.45 (s, 3H), |
| | | | 2.74 (s, 3H). |
| 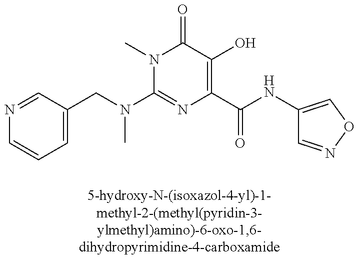 [SMILES: CN(Cc1cccnc1)c1nc(C(=O)Nc2cnoc2)c(O)c(=O)n1C] | 32 | ESI-MS m/Z = 357.3 [M + H]+. Calculated MW: 356.1 | ¹H NMR (300 MHz, DMSO- d6) δ 11.31 (s, 1H), 10.70 (s, 1H), 9.30 (s, 1H), 8.91 (s, 1H), 8.61 (s, 1H), 8.49 (d, J = 4.5 Hz, 1H), 7.82 (d, J = 7.9 Hz, 1H), 7.38 (dd, J = 7.9, 4.7 Hz, 1H), 4.44 (s, 2H), 3.48 (s, 3H), 2.73 (s, 3H). |
| 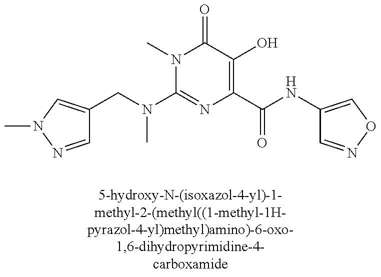 [SMILES: CN(Cc1cnn(C)c1)c1nc(C(=O)Nc2cnoc2)c(O)c(=O)n1C] | 33 | ESI-MS m/Z = 360.3 [M + H]+. Calculated MW: 359.1 | ¹H NMR (400 MHz, DMSO- d6) δ 11.23 (s, 1H), 10.66 (s, 1H), 9.30 (d, J = 1.9 Hz, 1H), 8.91 (d, J = 60 Hz, 1H), 7.70 (s, 1H), 7.41 (s, 1H), 4.14 (s, 2H), 3.80 (s, 3H), 3.47 (s, 3H), 2.72 (s, 3H). |
| 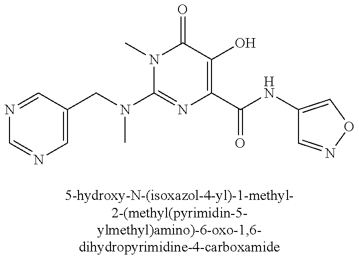 [SMILES: CN(Cc1cncnc1)c1nc(C(=O)Nc2cnoc2)c(O)c(=O)n1C] | 34 | ESI-MS m/Z = 358.1 [M + H]+. Calculated MW: 357.1 | 1H NMR (300 MHz, DMSO- d6) δ: 11.20 (s, 1H), 10.78 (s, 1H), 9.30 (s, 1H), 9.09 (s, 1H), 8.90-8.86 (m, 3H), 4.47 (s, 2H), 3.64 (s, 3H), 2.76 (s, 3H). |
| 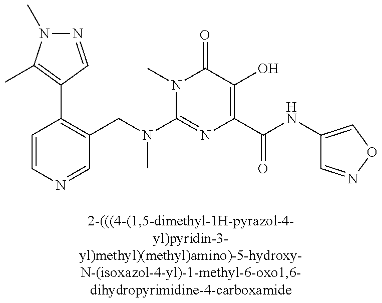 [SMILES: Cc1c(-c2ccncc2CN(C)c2nc(C(=O)Nc3cnoc3)c(O)c(=O)n2C)cnn1C] | 35 | ESI-MS m/Z = 451.2 [M + H]+. Calculated MW: 450.2 | ¹H NMR (400 MHz, DMSO- d6) δ 11.17 (brs, 1H), 10.47 (brs, 1H), 9.30 (s, 1H), 8.89 (s, 1H), 8.70 (s, 1H), 8.49 (d, J = 4.9 Hz, 1H), 7.52 (s, 1H), 7.15 (d, J = 5.0 Hz, 1H), 4.52 (s, 2H), 3.69 (s, 3H), 2.59 (s, 3H), 2.11 (s, 3H). |
| 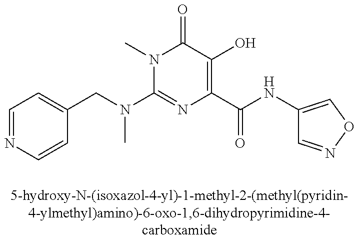 [SMILES: CN(Cc1ccncc1)c1nc(C(=O)Nc2cnoc2)c(O)c(=O)n1C] | 37 | ESI-MS m/Z = 357.2 [M + H]+. Calculated MW: 356.2 | ¹H NMR (300 MHz, DMSO- d) δ 11.21 (s, 1H), 10.48 (s, 1H), 9.31 (s, 1H), 8.91 (s, 1H), 8.63-8.39 (m, 2H), 7.52- 7.27 (m, 2H), 4.48 (s, 2H), 3.49 (s, 3H), 2.78 (s, 3H). |
| 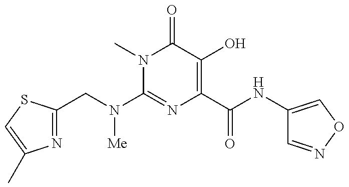 [SMILES: Cc1csc(CN(C)c2nc(C(=O)Nc3cnoc3)c(O)c(=O)n2C)n1] | 38 | ESI-MS m/Z = 377.2 [M + H]+. Calculated MW: 376.2 | ¹H NMR (300 MHz, DMSO- d₆) δ 11.18 (s, 1H), 10.53 (s, 1H), 9.31 (s, 1H), 8.90 (s, 1H), 7.23 (d, J = 1.0 Hz, 1H), 4.75 (s, 2H), 3.52 (s, 3H), 2.86 (s, 3H), 2.34 (d, J = 1.0 |
| | | | Hz, 3H). |
| 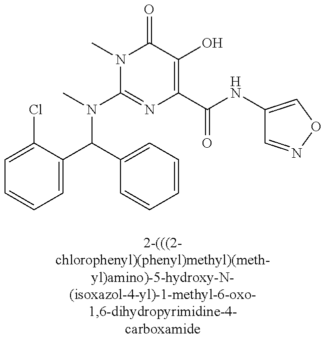 [SMILES: CN(c1nc(C(=O)Nc2cnoc2)c(O)c(=O)n1C)C(c1ccccc1)c1ccccc1Cl] | 39 From Isomer 1 Eutomer | ESI-MS m/Z = 466.1 [M + H]+. Calculated MW: 465.1 | ¹H NMR (400 MHz, DMSO- d6) δ 11.00 (s, 1H), 10.13 (s, 1H), 9.30 (s, 1H), 8.93 (s, 1H), 7.69 (dd, J = 7.8, 1.7 Hz, 1H), 7.62-7.51 (m, 2H), 7.40- 7.27 (m, 4H), 7.27-7.12 (m, 2H), 6.22 (s, 1H), 3.64 (s, 3H), 2.62 (s, 3H). |
| 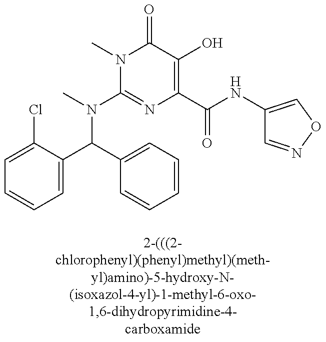 [SMILES: CN(c1nc(C(=O)Nc2cnoc2)c(O)c(=O)n1C)C(c1ccccc1)c1ccccc1Cl] | 40 From Isomer 2 Distomer | ESI-MS m/Z = 466.1 [M + H]+. Calculated MW: 465.1 | ¹H NMR (400 MHz, DMSO- d6) δ 11.00 (s, 1H), 10.20 (s, 1H), 9.30 (s, 1H), 8.92 (s, 1H), 7.73-7.65 (m, 1H), 7.57 (d, J = 7.5 Hz, 2H), 7.43-7.13 (m, 6H), 6.21 (s, 1H), 3.64 (s, 3H), 2.61 (s, 3H). |
| 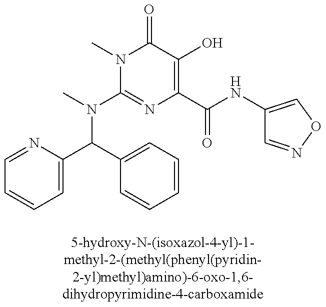 [SMILES: CN(c1nc(C(=O)Nc2cnoc2)c(O)c(=O)n1C)C(c1ccccc1)c1ccccn1] | 41 From Isomer 1 Distomer | ESI-MS m/Z = 433.1 [M + H]+. Calculated MW: 432.2 N- CHIRAL- PAK 1H (Lot No. IH30CS- WA011) column (4.6*100 mm, 3 um) SFC MeOH | ¹H NMR (400 MHz, DMSO- d₆) δ 11.01 (brs, 1H), 10.15 (brs, 1H), 9.29 (s, 1H), 8.96 (s, 1H), 8.44-8.43 (m, 1H), 7.71 (td, J = 7.7, 1.9 Hz, 1H), 7.62 (d, J = 7.9 Hz, 1H), 7.59-7.52 (m, 2H), 7.29 (dd, J = 8.3, 7.0 Hz, 2H), 7.21- 7.12 (m, 3H), 5.98 (s, 1H), 3.67 (s, 3H), 2.62 (s, 3H). |
| | | (0.2% | |
| | | MSA) | |
| | | 50:50 | |
| | | ratio | |
| | | flow rate = | |
| | | 2.0 | |
| | | mL/min | |
| | | Retention | |
| | | Time = | |
| | | 2.14 min | |
| 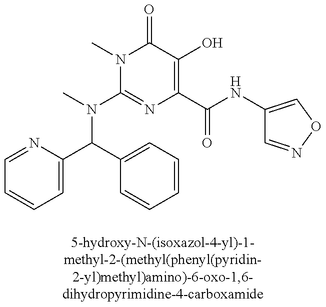 [SMILES: CN(c1nc(C(=O)Nc2cnoc2)c(O)c(=O)n1C)C(c1ccccc1)c1ccccn1] | 42 From Isomer 2 Eutomer | ESI-MS m/Z = 433.1 [M + H]+. Calculated MW: 432.2 | ¹H NMR (400 MHz, DMSO- d₆) δ 11.01 (brs, 1H), 10.15 (brs, 1H), 9.29 (s, 1H), 8.96 (s, 1H), 8.44-8.43 (m, 1H), 7.71 (td, J = 7.7, 1.9 Hz, 1H), 7.62 (d, J = 7.9 Hz, 1H), 7.59-7.52 (m, 2H), 7.29 (dd, J = 8.3, 7.0 Hz, 2H), 7.21- 7.12 (m, 3H), 5.98 (s, 1H), 3.67 (s, 3H), 2.62 (s, 3H). |
| 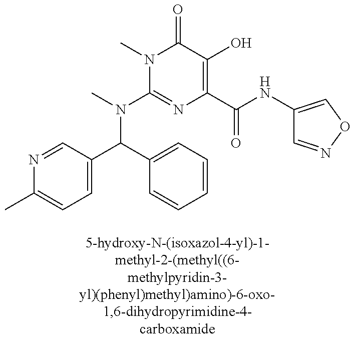 [SMILES: Cc1ccc(C(c2ccccc2)N(C)c2nc(C(=O)Nc3cnoc3)c(O)c(=O)n2C)cn1] | 43 From Isomer 1 distomer | ESI-MS m/Z = 447.2 [M + H]+. Calculated MW: 446.2 CHIRAL- PAK IB- N3 column (0.3* 10 cm, 3 um) SFC MeOH (0.2% MSA) 10- >50% flow rate = 2.0 mL/min | ¹H NMR (400 MHz, DMSO- d6) δ 9.27 (s, 1H), 8.91 (s, 1H), 8.54 (s, 1H), 7.76 (s, 2H), 7.54 (s, 3H), 7.29 (s, 3H), 7.19-7.08 (m, 3H), 5.86 (s, 2H), 3.75 (s, 2H), 3.64 (s, 3H), 2.54 (s, 4H), 2.33 (s, 3H), 1.24 (s, 1H). |
| | | Retention | |
| | | Time = | |
| | | 2.62 min | |
| 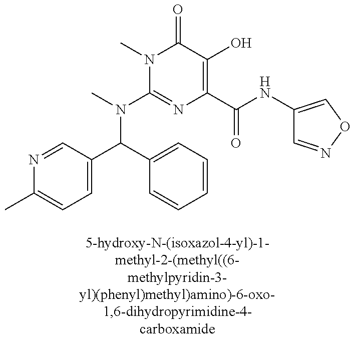 [SMILES: Cc1ccc(C(c2ccccc2)N(C)c2nc(C(=O)Nc3cnoc3)c(O)c(=O)n2C)cn1] | 44 From Isomer 2 eutomer | ESI-MS m/Z = 447.2 [M + H]+. Calculated MW: 447.2 CHIRAL- PAK IB- N3 column (0.3* 10 cm, 3 um) SFC MeOH (0.2% MSA) 10- >50% flow rate = 2.0 mL/min | ¹H NMR (400 MHz, DMSO- d₆) δ 11.10 (s, 1H), 10.28 (s, 1H), 9.31 (s, 1H), 8.97 (s, 1H), 8.57 (d, J = 2.6 Hz, 1H), 7.81 (d, J = 7.9 Hz, 1H), 7.59- 7.47 (m, 2H), 7.32 (t, J = 7.6 Hz, 2H), 7.19 (dd, J = 8.4, 6.7 Hz, 2H), 5.96 (s, 1H), 3.65 (s, 3H), 2.60 (s, 3H), 2.37 (s, 3H). |
| | | Retention | |
| | | Time = | |
| | | 2.37 min | |
| 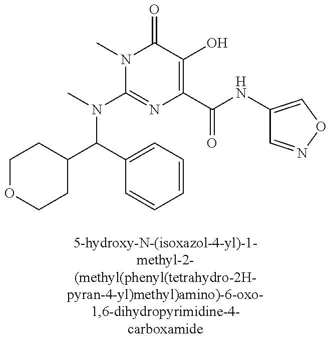 [SMILES: CN(c1nc(C(=O)Nc2cnoc2)c(O)c(=O)n1C)C(c1ccccc1)C1CCOCC1] | 45 From Isomer 1 Distomer | ESI-MS m/Z = 440.3 [M + H]+. Calculated MW: 439.2 CHIRAL- PAK IB- N3 column (0.3*10 cm, 3 um) SFC MeOH (0.2% MSA) 10- >50% flow rate = 2.0 mL/min | ¹H NMR (400 MHz, DMSO- d6) δ 11.04 (s, 1H), 10.10 (s, 1H), 9.26 (s, 1H), 8.87 (d, J = 1.1 Hz, 1H), 7.32-7.25 (m, 2H), 7.26-7.16 (m, 3H), 4.46 (d, J = 9.0 Hz, 1H), 3.91 (dd, J = 11.3, 4.0 Hz, 1H), 3.78 (d, J = 11.1 Hz, 1H), 3.59 (s, 3H), 3.39 (s, 1H), 3.26 (s, 2H), 2.73 (s, 3H), 1.86 (d, J = 12.8 Hz, 1H), 1.32 (td, J = |
| | | retention | 12.3, 4.3 Hz, |
| | | Time = | 1H), 1.20 (d, J = |
| | | 2.37 min | 14.6 Hz, 2H). |
| 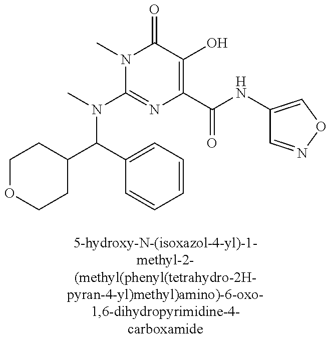 [SMILES: CN(c1nc(C(=O)Nc2cnoc2)c(O)c(=O)n1C)C(c1ccccc1)C1CCOCC1] | 46 From Isomer 2 eutomer | ESI-MS m/Z = 440.3 [M + H]+. Calculated MW: 439.2 CHIRAL- PAK IB- N3 column (0.3*10 cm, 3 um) SFC MeOH (0.2% MSA) 10- >50% flow rate = 2.0 mL/min | ¹H NMR (300 MHz, DMSO- d₆) δ 11.08 (brs, 1H), 10.18 (brs, 1H), 9.26 (s, 1H), 8.87 (s, 1H), 7.19-7.33 (m, 5H), 4.43 (d, J = 9.0 Hz, 1H), 3.92 (d, J = 11.3 Hz, 1H), 3.80 (d, J = 11.1 Hz, 1H), 3.59 (s, 3H), 3.38 (d, J = 11.5 Hz, 1H), 2.73 (s, 3H), 1.88 (d, J = 13.0 Hz, 1H), 1.36-1.21 (m, 3H). |
| | | Retention | |
| | | Time = | |
| | | 2.37 min | |
| 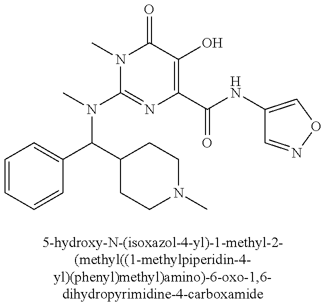 [SMILES: CN1CCC(C(c2ccccc2)N(C)c2nc(C(=O)Nc3cnoc3)c(O)c(=O)n2C)CC1] | 49 | ESI-MS m/z = 453.2 [M + H]+. Calculated MW: 452.2 | 1H NMR (400 MHz, DMSO- d6) δ 12.00- 11.03 (m, 1H), 9.28 (s, 1H), 8.92 (s, 1H), 8.39 (s, 1H), 7.58 (d, J = 7.6 Hz, 2H), 7.39- 7.20 (m, 4H), 6.82 (s, 1H), 5.15 (s, 1H), 3.60-3.55 (m, 3H), 3.27-3.19 (m, 6H), 3.17- 3.03 (m, 8H), 2.18 (d, J = 15.6 Hz, 2H), 1.80- 1.56 (m, 2H), |
| | | | 1.26 (d, J = 14.6 |
| | | | Hz, 1H). |
| 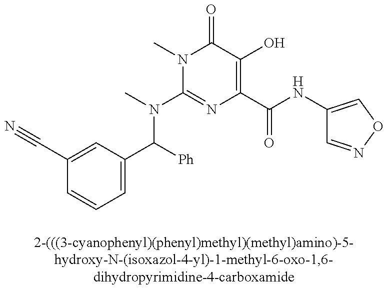 [SMILES: CN(c1nc(C(=O)Nc2cnoc2)c(O)c(=O)n1C)C(c1ccccc1)c1cccc(C#N)c1] | 50 From Isomer 1 (R) Eutomer | ESI-MS m/z = 457.2 [M + H]⁺. Calculated MW = 456.2 CHIRAL- PAK IB N-3 (Lot No. IBN3SCK- VD005) column (3.0*100 mm, 3 um) SFC | ¹H NMR (300 MHz, DMSO- d₆) δ 11.07 (s, 1H), 10.22 (s, 1H), 9.31 (s, 1H), 8.96 (s, 1H), 8.03 (s, 1H), 7.84 (d, J = 7.8 Hz, 1H), 7.60 (t, J = 8.4 Hz, 3H), 7.47 (t, J = 7.8 Hz, 1H), 7.33 (t, J = 7.5 Hz, 2H), 7.21 (t, J = 7.3 Hz, 1H), 5.99 (s, 1H), 3.68 (s, 3H), 2.61 (s, 3H). |
| | | MeOH | |
| | | (0.2% | |
| | | MSA) | |
| | | 90:10 | |
| | | ratio | |
| | | flow rate = | |
| | | 2.0 | |
| | | mL/min | |
| | | Retention | |
| | | Time = | |
| | | 2.67 min | |
| 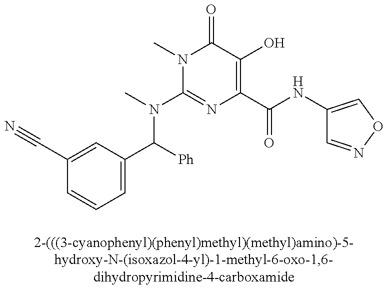 [SMILES: CN(c1nc(C(=O)Nc2cnoc2)c(O)c(=O)n1C)C(c1ccccc1)c1cccc(C#N)c1] | 51 From Isomer 2 (S) distomer | ESI-MS m/Z = 457.2 [M + H]+. Calculated MW: 456.2 | ¹H NMR (300 MHz, DMSO- d₆) δ 11.07 (s, 1H), 10.24 (s, 1H), 9.30 (s, 1H), 8.96 (s, 1H), 8.03 (s, 1H), 7.83 (d, J = 7.9 Hz, 1H), 7.59 (t, J = 8.2 Hz, 3H), 7.47 (t, J = 7.7 Hz, 1H), 7.33 (t, J = 7.5 Hz, 2H), 7.21 (t, J = 7.3 Hz, 1H), 5.99 (s, 1H), 3.68 (s, 3H), 2.61 (s, 3H). |
| 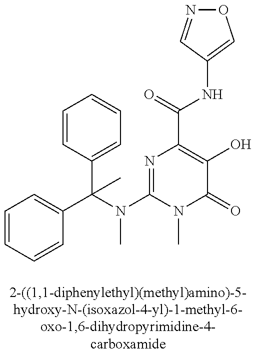 [SMILES: CN(c1nc(C(=O)Nc2cnoc2)c(O)c(=O)n1C)C(C)(c1ccccc1)c1ccccc1] | 52 | ESI-MS m/Z = 446.2 [M + H]+. Calculated MW: 445.2 | 1H NMR (400 MHz, DMSO- d6) δ 9.88 (s, 1H), 9.19 (s, 1H), 8.78 (s, 1H), 7.62-6.99 (m, 17H), 3.43 (s, 3H), 2.67 (s, 3H), 2.33 (s, 3H), 2.29 (s, 3H), 1.20 (t, J = 7.1 Hz, 3H). |
| 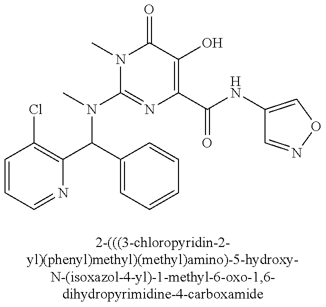 [SMILES: CN(c1nc(C(=O)Nc2cnoc2)c(O)c(=O)n1C)C(c1ccccc1)c1ncccc1Cl] | 53 From Isomer 1 eutomer | ESI-MS m/Z = 467.1 [M + H]+. Calculated MW: 466.2 CHIRAL- PAK IH- 3 column (0.46*10 cm, 3 um) SFC MeOH (0.2% MSA) 10- >50% flow rate = 2.0 | 1H NMR (300 MHz, DMSO- d6) δ 10.89 (brs, 1H), 10.12 (brs, 1H), 9.28 (s, 1H), 8.90 (s, 1H), 8.44 (dd, J = 4.6, 1.5 Hz, 1H), 7.81 (dd, J = 8.1, 1.5 Hz, 1H), 7.61-7.59 (m, 2H), 7.38 (dd, J = 8.3, 6.5 Hz, 2H), 7.35- 7.26 (m, 1H), 7.20 (dd, J = 8.1, 4.6 Hz, 1H), 6.31 (s, 1H), 3.64 (s, |
| | | mL/min | 3H), 2.63 (s, |
| | | Retention | 3H). |
| | | Time = | |
| | | 4.07 min | |
| 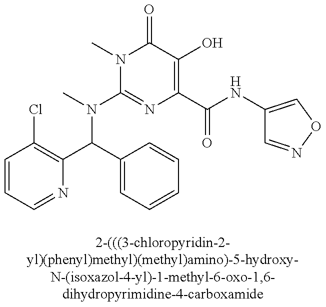 [SMILES: CN(c1nc(C(=O)Nc2cnoc2)c(O)c(=O)n1C)C(c1ccccc1)c1ncccc1Cl] | 54 From Isomer 2 distomer | ESI-MS m/Z = 467.1 [M + H]+. Calculated MW: 466.2 CHIRAL- PAK IH- 3 column (0.46*10 cm, 3 um) SFC MeOH (0.2% MSA) 10- >50% flow rate = 2.0 mL/min Retention Time = 3.19 min | 1H NMR (400 MHz, DMSO- d6) δ 10.90 (brs, 1H), 10.16 (brs, 1H), 9.28 (s, 1H), 8.89 (s, 1H), 8.44 (dd, J = 4.6, 1.5 Hz, 1H), 7.81 (dd, J = 8.1, 1.5 Hz, 1H), 7.61-7.59 (m, 2H), 7.39- 7.35 (m, 2H), 7.35-7.27 (m, 1H), 7.20 (dd, J = 8.1, 4.6 Hz, 1H), 6.30 (s, 1H), 3.64 (s, 3H), 2.63 (s, 3H). |
| 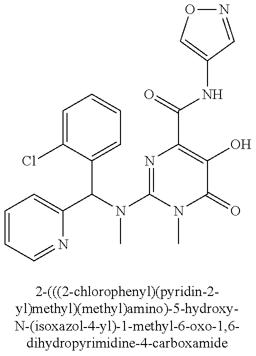 [SMILES: CN(c1nc(C(=O)Nc2cnoc2)c(O)c(=O)n1C)C(c1ccccn1)c1ccccc1Cl] | 56 From Isomer 1 Eutomer | ESI-MS m/Z = 467.2 [M + H]+. Calculated MW: 466.2 CHIRAL- PAK IH- 3 column (0.46*10 cm, 3 um) SFC MeOH (0.2% MSA) 10- >50% flow rate = 2.0 mL/min Retention Time = | ¹H NMR (400 MHz, DMSO- d6) δ 11.02 (s, 1H), 10.11 (s, 1H), 9.29 (d, J = 0.9 Hz, 1H), 8.91 (d, J = 1.0 Hz, 1H), 8.51- 8.43 (m, 1H), 7.76 (td, J = 7.6, 1.8 Hz, 1H), 7.73-7.67 (m, 2H), 7.41 (dd, J = 7.9, 1.4 Hz, 1H), 7.34 (td, J = 7.6, 1.4 Hz, 1H), 7.29-7.19 (m, 2H), 6.31 (s, 1H), 3.59 (s, 3H), 2.66 (s, 3H). |
| | | 4.21 min | |
| 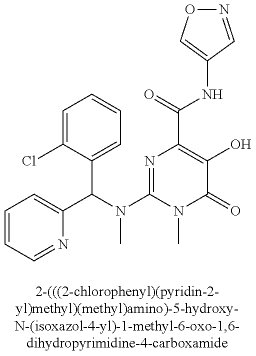 [SMILES: CN(c1nc(C(=O)Nc2cnoc2)c(O)c(=O)n1C)C(c1ccccn1)c1ccccc1Cl] | 57 From Isomer 2 distomer | ESI-MS m/Z = 467.2 [M + H]+. Calculated MW: 466.2 CHIRAL- PAK IH- 3 column (0.46*10 cm, 3 um) SFC MeOH (0.2% MSA) 10- >50% flow rate = 2.0 mL/min Retention Time = | ¹H NMR (400 MHz, DMSO- d6) δ 11.02 (s, 1H), 10.11 (s, 1H), 9.29 (d, J = 0.9 Hz, 1H), 8.91 (d, J = 1.0 Hz, 1H), 8.51- 8.43 (m, 1H), 7.76 (td, J = 7.6, 1.8 Hz, 1H), 7.73-7.67 (m, 2H), 7.41 (dd, J = 7.9, 1.4 Hz, 1H), 7.34 (td, J = 7.6, 1.4 Hz, 1H), 7.29-7.19 (m, 2H), 6.31 (s, 1H), 3.59 (s, 3H), 2.66 (s, 3H). |
| | | 4.88 min | |
| 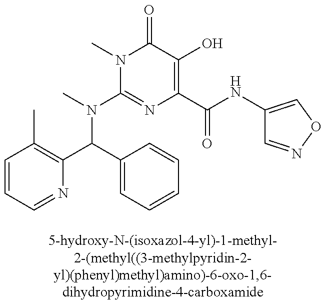 [SMILES: Cc1cccnc1C(c1ccccc1)N(C)c1nc(C(=O)Nc2cnoc2)c(O)c(=O)n1C] | 58 From Isomer 1 distomer | ESI-MS m/Z = 447.1 [M + H]+. Calculated MW: 446.2 N- CHIRAL- PAK IB N-3 (Lot No. IBN3SCK- VD005) column (3.0*100 mm, 3.0 um) SFC | ¹H NMR (400 MHz, DMSO- d₆) δ 10.97 (s, 1H), 10.37 (s, 1H), 9.27 (s, 1H), 8.87 (s, 1H), 8.29 (dd, J = 4.9, 1.7 Hz, 1H), 7.57-7.51 (m, 2H), 7.46- 7.39 (m, 1H), 7.35 (t, J = 7.6 Hz, 2H), 7.31- 7.22 (m, 1H), 7.03 (dd, J = 7.6, 4.7 Hz, 1H), 6.04 (s, 1H), 3.65 (s, 3H), 2.60 (s, |
| | | MeOH | 3H), 2.37 (s, |
| | | (0.2% MS | 3H). |
| | | A) | |
| | | 90:10 | |
| | | ratio | |
| | | flow rate = | |
| | | 2 mL/min | |
| | | Time = | |
| | | 1.63 min | |
| 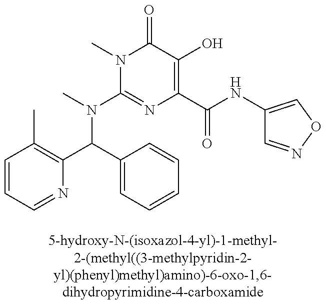 [SMILES: Cc1cccnc1C(c1ccccc1)N(C)c1nc(C(=O)Nc2cnoc2)c(O)c(=O)n1C] | 59 From Isomer 2 eutomer | ESI-MS m/Z = 447.1 [M + H]+. Calculated MW: 446.2 N- CHIRAL- PAK IB N-3 (Lot No. IBN3SCK- VD005) column (3.0*100 mm, 3.0 um) SFC | 1H NMR (400 MHz, DMSO- d₆) δ 10.97 (s, 1H), 10.36 (s, 1H), 9.27 (s, 1H), 8.87 (s, 1H), 8.29 (dd, J = 4.7, 1.7 Hz, 1H), 7.57-7.50 (m, 2H), 7.46- 7.39 (m, 1H), 7.35 (t, J = 7.5 Hz, 2H), 7.31- 7.22 (m, 1H), 7.03 (dd, J = 7.6, 4.7 Hz, 1H), 6.04 (s, 1H), 3.65 (s, 3H), 2.60 (s, |
| | | MeOH(0.2% | 3H), 2.37 (s, |
| | | MSA) | 3H). |
| | | 90:10 | |
| | | ratio | |
| | | flow rate = | |
| | | 2 | |
| | | mL/min | |
| | | Time = | |
| | | 1.05 min | |
| 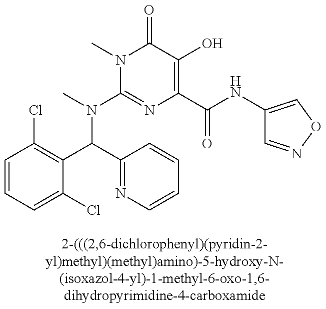 [SMILES: CN(c1nc(C(=O)Nc2cnoc2)c(O)c(=O)n1C)C(c1ccccn1)c1c(Cl)cccc1Cl] | 60 From Isomer 1 distomer | ESI-MS m/Z = 501.1 [M + H]+. Calculated MW: 500.1 CHIRAL- PAK IB N-3 (Lot No. IBN3SCK- VD005) column (3.0*100 mm, 3.0 um) SFC MeOH | ¹H NMR (300 MHz, DMSO- d₆) δ 11.11 (s, 1H), 10.07 (s, 1H), 9.26 (s, 1H), 8.82 (s, 1H), 8.47 (dt, J = 4.7, 1.6 Hz, 1H), 7.83 (td, J = 7.7, 1.8 Hz, 1H), 7.61 (d, J = 8.1 Hz, 1H), 7.48 (d, J = 7.9 Hz, 2H), 7.38- 7.26 (m, 2H), 6.59 (s, 1H), 3.54 (s, 3H), 2.84 (s, 3H). |
| | | (0.2% MS | |
| | | A) 75:25 | |
| | | ratio | |
| | | flow rate = | |
| | | 2 | |
| | | mL/min | |
| | | Time = | |
| | | 2.86 min | |
| 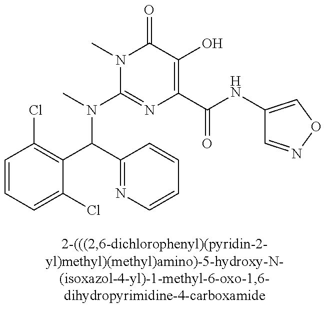 [SMILES: CN(c1nc(C(=O)Nc2cnoc2)c(O)c(=O)n1C)C(c1ccccn1)c1c(Cl)cccc1Cl] | 61 From Isomer 2 eutomer | ESI-MS m/Z = 501.1 [M + H]+. Calculated MW: 500.1 CHIRAL- PAK IB N-3 (Lot No. IBN3SCK- VD005) column (3.0*100 mm, 3.0 um) SFC MeOH | ¹H NMR (300 MHz, DMSO- d₆) δ 11.11 (s, 1H), 10.05 (s, 1H), 9.26 (s, 1H), 8.82 (s, 1H), 8.48-8.47 (m, 1H), 7.86 (td, J = 7.8, 1.8 Hz, 1H), 7.61 (d, J = 7.9 Hz, 1H), 7.48 (d, J = 8.0 Hz, 2H), 7.41-7.26 (m, 2H), 6.59 (s, 1H), 3.54 (s, 3H), 2.84 (s, 3H). |
| | | (0.2% MSA) | |
| | | 75:25 | |
| | | ratio | |
| | | flow rate = | |
| | | 2 | |
| | | mL/min | |
| | | Time = | |
| | | 3.42 min | |
| 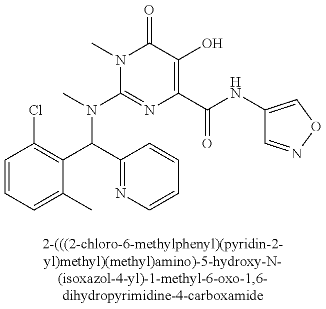 [SMILES: Cc1cccc(Cl)c1C(c1ccccn1)N(C)c1nc(C(=O)Nc2cnoc2)c(O)c(=O)n1C] | 62 From Isomer 1 eutomer | ESI-MS m/Z = 481.2 [M + H]⁺. Calculated MW: 480.1 CHIRAL- PAK IH column (0.46*10 cm, 3 um) SFC MeOH (0.2% MSA) 25% flow rate = 2.0 | ¹H NMR (400 MHz, DMSO- d6) δ 10.98 (s, 1H), 9.88 (s, 1H), 9.24 (s, 1H), 8.81 (s, 1H), 8.38 (dt, J = 4.6, 1.6 Hz, 1H), 7.69 (td, J = 7.7, 1.8 Hz, 1H), 7.36 (dd, J = 15.9, 8.0 Hz, 2H), 7.28-7.06 (m, 3H), 6.72 (s, 1H), 3.65 (s, 3H), 2.63 (s, 3H), 2.44 (s, 3H). |
| | | mL/min | |
| | | Retention | |
| | | Time = | |
| | | 4.80 min | |
| 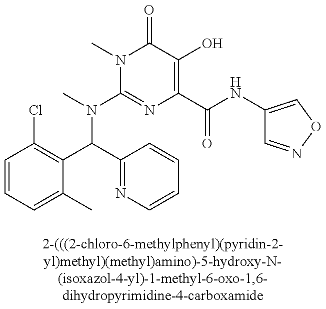 [SMILES: Cc1cccc(Cl)c1C(c1ccccn1)N(C)c1nc(C(=O)Nc2cnoc2)c(O)c(=O)n1C] | 63 From Isomer 2 distomer | ESI-MS m/Z = 481.2 [M + H]⁺. Calculated MW: 480.1 CHIRAL- PAK IH column (0.46*10 cm, 3 um) SFC MeOH (0.2% MSA) 25% flow rate = 2.0 | ¹H NMR (400 MHz, DMSO- d6) δ 10.98 (s, 1H), 9.88 (s, 1H), 9.24 (s, 1H), 8.81 (s, 1H), 8.46-8.29 (m, 1H), 7.69 (td, J = 7.7, 1.8 Hz, 1H), 7.36 (dd, J = 16.4, 7.9 Hz, 2H), 7.29-7.01 (m, 3H), 6.72 (s, 1H), 3.65 (s, 3H), 2.63 (s, 3H), 2.44 (s, 3H). |
| | | mL/min | |
| | | Retention | |
| | | Time = | |
| | | 5.29 min | |
| 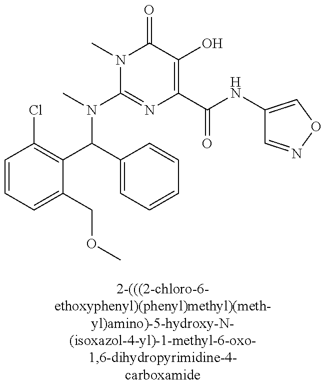 [SMILES: COCc1cccc(Cl)c1C(c1ccccc1)N(C)c1nc(C(=O)Nc2cnoc2)c(O)c(=O)n1C] | 66 From Isomer 1 distomer | ESI-MS m/Z = 510.1 [M + H]+. Calculated MW: 509.2 CHIRAL- PAK IH- 3 column (0.46*10 cm, 3 um) Mobile Phase: MtBE (0.1% TFA):EtOH = 70:30 flow rate = 1.0 mL/min Retention Time = 2.78 min | ¹H NMR (300 MHz, DMSO- d6) δ 10.26- 9.96 (brs, 1H), 9.15 (s, 1H), 8.78 (s, 1H), 7.49 (d, J = 7.6 Hz, 2H), 7.32 (t, J = 7.4 Hz, 2H), 7.27-7.16 (m, 2H), 7.01 (d, J = 7.8 Hz, 1H), 6.94 (d, J = 8.4 Hz, 1H), 6.39 (s, 1H), 4.10- 3.98 (m, 1H), 3.89-3.60 (m, 1H), 3.56 (s, 3H), 2.75 (s, 3H), 1.14 (t, J = 6.9 Hz, 3H). |
| 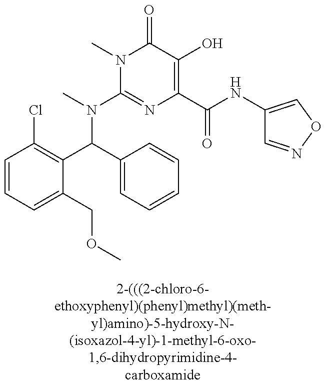 [SMILES: COCc1cccc(Cl)c1C(c1ccccc1)N(C)c1nc(C(=O)Nc2cnoc2)c(O)c(=O)n1C] | 67 From Isomer 2 eutomer | ESI-MS m/Z = 510.1 [M + H]+. Calculated MW: 509.2 CHIRAL- PAK IH- 3 column (0.46*10 cm, 3 um) Mobile Phase: MtBE (0.1% TFA):EtOH = 70:30 flow rate = 1.0 mL/min Retention Time = 3.33 min | ¹H NMR (300 MHz, DMSO- d6) δ 10.05 (brs, 1H), 9.16 (s, 1H), 8.79 (s, 1H), 7.50 (d, J = 7.6 Hz, 2H), 7.32 (t, J = 7.5 Hz, 2H), 7.22 (t, J = 7.8 Hz, 2H), 6.98 (dd, J = 21.2, 8.1 Hz, 2H), 6.38 (s, 1H), 4.04 (dd, J = 9.8, 7.0 Hz, 1H), 3.83 (dd, J = 9.8, 6.9 Hz, 1H), 3.56 (s, 3H), 2.76 (s, 3H), 1.13 (t, J = 7.0 Hz, 3H). |
| 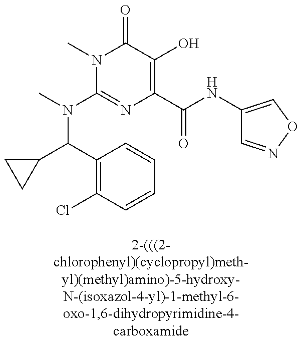 [SMILES: CN(c1nc(C(=O)Nc2cnoc2)c(O)c(=O)n1C)C(c1ccccc1Cl)C1CC1] | 68 From Isomer 1 eutomer | ESI-MS m/z = 430.1 [M + H]+. Calculated MW = 429.1 Amylose- C Neo column (0.46*50 cm, 3 um) Mobile Phase: Hex(0.3% TFA):IPA = 50:50 flow rate = 1.0 mL/min Retention Time = 2.50 min | ¹H NMR (400 MHz, DMSO- d6) δ 11.04 (s, 1H), 10.16 (s, 1H), 9.30 (s, 1H), 8.91 (s, 1H), 7.66-7.59 (m, 1H), 7.36- 7.25 (m, 2H), 7.22-7.14 (m, 1H), 4.37 (d, J = 9.7 Hz, 1H), 3.58 (s, 3H), 2.87 (s, 3H), 1.41-1.31 (m, 1H), 0.76 (s, 1H), 0.65-0.56 (m, 1H), 0.44- 0.25 (m, 2H). |
| 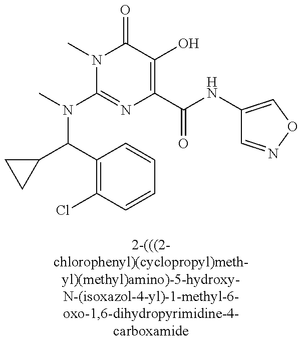 [SMILES: CN(c1nc(C(=O)Nc2cnoc2)c(O)c(=O)n1C)C(c1ccccc1Cl)C1CC1] | 69 From Isomer 2 distomer | ESI-MS m/z = 430.1 [M + H]+. Calculated MW = 429.1 Amylose- C Neo column (0.46*50 cm, 3 um) Mobile Phase: Hex(0.3% TFA):IPA = 50:50 flow rate = 1.0 mL/min Retention Time = | ¹H NMR (400 MHz, DMSO- d6) δ 11.04 (s, 1H), 10.17 (s, 1H), 9.30 (s, 1H), 8.91 (d, J = 1.2 Hz, 1H), 7.63 (dd, J = 7.8, 1.7 Hz, 1H), 7.37-7.24 (m, 2H), 7.22- 7.14 (m, 1H), 4.36 (d, J = 9.7 Hz, 1H), 3.58 (s, 3H), 2.87 (s, 3H), 1.37 (d, J = 8.8 Hz, 1H), 0.76 (s, 1H), 0.64-0.56 (m, 1H), 0.47-0.20 (m, 2H). |
| | | 1.85 min | |
| 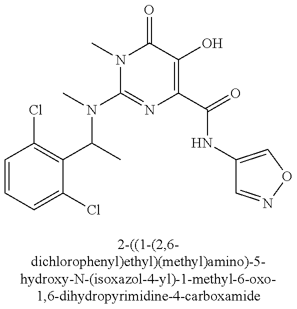 [SMILES: CC(c1c(Cl)cccc1Cl)N(C)c1nc(C(=O)Nc2cnoc2)c(O)c(=O)n1C] | 70 From Isomer 1 Distomer | ESI-MS m/Z = 438.2 [M + H]+. Calculated MW: 437.1 Chiral Cellulose SB column (0.46*10 cm, 3 um) MTBE (0.1% FA)/EtOH - 70:30 flow rate = 1.0 | ¹H NMR (400 MHz, DMSO- d₆) δ 10.98 (s, 1H), 10.09 (s, 1H), 9.32 (s, 1H), 8.93 (s, 1H), 7.40 (d, J = 8.0 Hz, 2H), 7.22 (t, J = 8.0 Hz, 1H), 5.62 (q, J = 6.9 Hz, 1H), 3.54 (s, 3H), 2.74 (s, 3H), 1.61 (d, J = 7.0 Hz, 3H). |
| | | mL/min | |
| | | Retention | |
| | | Time = | |
| | | 2.11 min | |
| 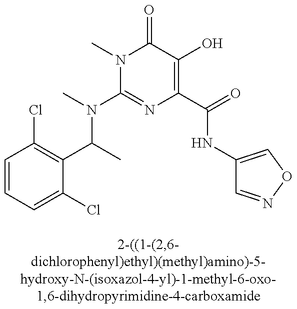 [SMILES: CC(c1c(Cl)cccc1Cl)N(C)c1nc(C(=O)Nc2cnoc2)c(O)c(=O)n1C] | 71 From Isomer 2 eutomer | ESI-MS m/Z = 438.2 [M + H]+. Calculated MW: 437.1 Chiral Cellulose SB column (0.46*10 cm, 3 um) MTBE (0.1% FA)/EtOH - 70:30 flow rate = 1.0 | ¹H NMR (400 MHz, DMSO- d₆) δ 10.98 (s, 1H), 10.09 (s, 1H), 9.32 (s, 1H), 8.93 (s, 1H), 7.40 (d, J = 8.0 Hz, 2H), 7.22 (t, J = 8.0 Hz, 1H), 5.62 (q, J = 6.9 Hz, 1H), 3.54 (s, 3H), 2.74 (s, 3H), 1.61 (d, J = 6.9 Hz., 3H). |
| | | mL/min | |
| | | Retention | |
| | | Time = | |
| | | 2.34 min | |
| 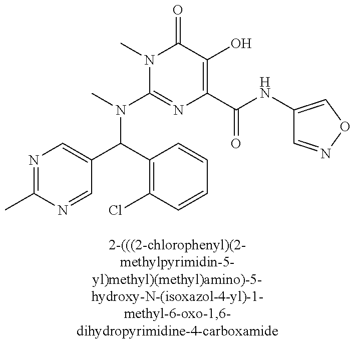 [SMILES: Cc1ncc(C(c2ccccc2Cl)N(C)c2nc(C(=O)Nc3cnoc3)c(O)c(=O)n2C)cn1] | 81 From Isomer 1 Distomer | ESI-MS m/Z = 482.7 [M + H]+. Calculated MW: 481.9 CHIRAL- PAK IH(250*21) mm, 5 u; Isocratic Line A:B (75:25): IPA:MeOH (50:50) in Hexanes + 0.1% DEA: | ¹H NMR (400 MHz, DMSO- d₆): δ 2.64 (s, 3H), 3.37 (s, 3H), 3.62 (s, 3H), 6.25 (s, 1H), 7.26-7.40 (m, 3H), 7.82 (d, J = 6.0 Hz, 1H), 8.13 (s, 1H), 8.76-8.83 (m, 3H), 10.35 (bs, 1H), 11.12 (bs, 1H). |
| | | Column | |
| | | Flow: 20 | |
| | | ml/min; | |
| | | RT of | |
| | | Isomer- | |
| | | 1:18.402 | |
| 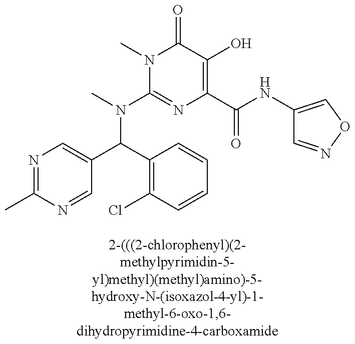 [SMILES: Cc1ncc(C(c2ccccc2Cl)N(C)c2nc(C(=O)Nc3cnoc3)c(O)c(=O)n2C)cn1] | 82 From Isomer 2 eutomer | ESI-MS m/Z = 482.3 [M + H]+. Calculated MW: 481.9 CHIRAL- PAK IH(250*21) mm, 5 u; Isocratic Line A:B (75:25); IPA:MeOH (50:50) in Hexanes + 0.1% DEA; Column | ¹H NMR (400 MHz, DMSO- d₆): δ 2.60 (s, 3H), 3.37 (s, 3H), 3.65 (s, 3H), 6.11 (s, 1H), 7.27-7.40 (m, 3H), 7.81 (d, J = 6.0 Hz, 1H), 8.14 (s, 1H), 8.76-8.82 (m, 3H), 9.99 (bs, 1H), 10.35 (s, 1H). |
| | | Flow: 20 | |
| | | ml/min; | |
| | | RT of | |
| | | Isomer- | |
| | | 2:23.020 | |
| 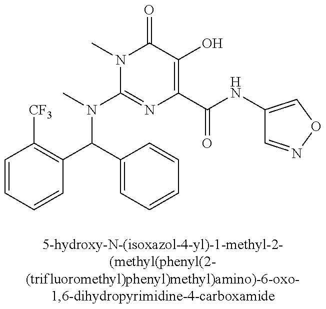 [SMILES: CN(c1nc(C(=O)Nc2cnoc2)c(O)c(=O)n1C)C(c1ccccc1)c1ccccc1C(F)(F)F] | 83 From Isomer 1 Distomer | ESI-MS m/Z = 500.7 [M + H]+. Calculated MW: 499.5 CHIRAL- PAK IH (250*21) mm, 5 u; Isocratic Line A:B (85:15); IPA:ACN (70:30) in hexanes + 0.1% | ¹H NMR (400 MHz, DMSO- d₆): δ 2.55 (s, 1H), 3.63 (s, 3H), 6.07 (s, 1H), 7.17 (t, J = 7.2 Hz, 1H), 7.27 (t, J = 7.6 Hz, 2H), 7.43- 7.49 (m, 3H), 7.66-7.72 (m, 2H), 8.11 (s, 1H), 8.85 (s, 1H), 9.26 (s, 1H), 10.22 (s, 1H), 11.04 (s, 1H). |
| | | DEA; | |
| | | Column | |
| | | Flow: 16 | |
| | | ml/min; | |
| | | RT of | |
| | | Isomer- | |
| | | 1:33.6 | |
| | | min. | |
| 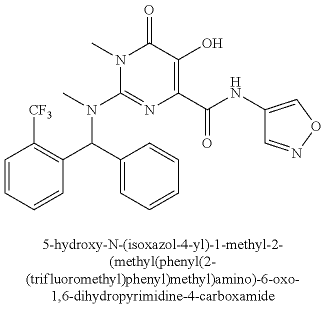 [SMILES: CN(c1nc(C(=O)Nc2cnoc2)c(O)c(=O)n1C)C(c1ccccc1)c1ccccc1C(F)(F)F] | 84 From Isomer 2 eutomer | ESI-MS m/Z = 500.7 [M + H]+. Calculated MW: 499.5 CHIRAL- PAK IH(250*21) mm, 5 u; Isocratic Line A:B (85:15); IPA:ACN (70:30) in hexanes + 0.1% DEA; | ¹H NMR (400 MHz, DMSO- d₆): δ 2.55 (s, 3H), 3.63 (s, 3H), 6.07 (s, 1H), 7.17 (t, J = 6.0 Hz, 1H), 7.27 (s, 2H), 7.44-7.49 (m, 3H), 7.66-7.70 (m, 2H), 8.11 (s, 1H), 8.85 (s, 1H), 9.27 (s, 1H), 10.13 (s, 1H), 11.02 (s, 1H). |
| | | Column | |
| | | Flow: 16 | |
| | | ml/min; | |
| | | RT of | |
| | | Isomer- | |
| | | 2: 40.5 | |
| | | min. | |
| 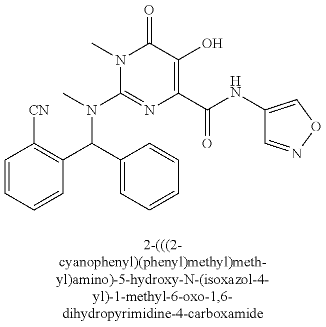 [SMILES: CN(c1nc(C(=O)Nc2cnoc2)c(O)c(=O)n1C)C(c1ccccc1)c1ccccc1C#N] | 85 From Isomer 1 Distomer | ESI-MS m/Z = 457.7 [M + H]+. Calculated MW: 456.5 CHIRAL- PAK IB- N(250*21) mm, 5 u; Isocratic Line A:B 75:25): IPA:MeOH (50:50) in Hexanes + 0.1% DEA; | ¹H NMR (400 MHz, DMSO- d₆): δ 2.66 (s, 3H), 3.72 (s, 3H), 6.17 (s, 1H), 7.31 (d, J = 6.8 Hz, 1H), 7.39-7.41 (m, 3H), 7.67 (d, J = 7.2 Hz, 3H), 7.77 (d, J = 7.6 Hz, 1H), 7.85 (d, J = 8.0 Hz, 1H), 8.90 (s, 1H), 9.31 (s, 1H), 10.54 (bs, 1H), 11.01 (bs, 1H). |
| | | Column | |
| | | Flow: 18 | |
| | | ml/min; | |
| | | RT of | |
| | | Isomer-1: | |
| | | 24.29 min. | |
| 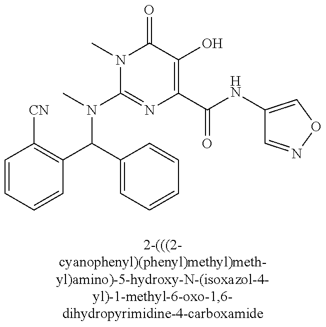 [SMILES: CN(c1nc(C(=O)Nc2cnoc2)c(O)c(=O)n1C)C(c1ccccc1)c1ccccc1C#N] | 86 From Isomer 2 eutomer | ESI-MS m/Z = 457.7 [M + H]+. Calculated MW: 456.5 CHIRAL- PAK IB- N(250*21) mm, 5 u; Isocratic Line A:B (75:25); IPA:MeOH (50:50) in Hexanes + 0.1% DEA; Column | ¹H NMR (400 MHz, DMSO- d₆) : δ 2.67 (s, 3H), 3.72 (s, 3H), 6.18 (s, 1H), 7.31-7.41 (m, 4H), 7.66- 7.85 (m, 5H), 8.91 (d, J = 2.8 Hz, 1H), 9.32 (d, J = 2.8 Hz, 1H), 10.39 (bs, 1H), 11.02 (bs, 1H). |
| | | Flow: 18 | |
| | | ml/min; | |
| | | RT of | |
| | | Isomer-2: | |
| | | 29.11 min. | |
| 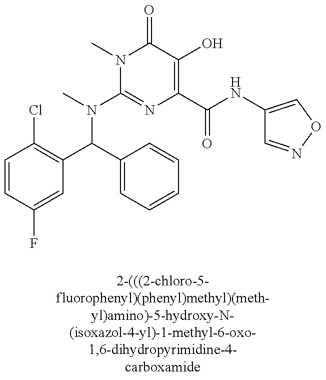 [SMILES: CN(c1nc(C(=O)Nc2cnoc2)c(O)c(=O)n1C)C(c1ccccc1)c1cc(F)ccc1Cl] | 87 From Isomer 1 Distomer | ESI-MS m/Z = 484.3 [M + H]+. Calculated MW: 483.9 CHIRAL- PAK AD- H(250*21) mm, 5 u; Isocratic: Line A:B (80:20); MeOH:ACN (50:50) in Liquid CO₂ + 0.1% DEA; Column Flow: 80 ml/min; RT of Isomer-1: | ¹H NMR (400 MHz, DMSO- d₆): δ 2.61 (s, 3H), 3.66 (s, 3H), 6.19 (s, 1H), 7.07-7.62 (m, 8H), 8.92 (s, 1H), 9.30 (s, 1H), 10.16 (s, 1H), 10.99 (s, 1H). |
| | | 2.37 min. | |
| 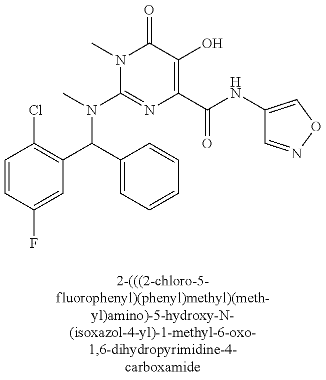 [SMILES: CN(c1nc(C(=O)Nc2cnoc2)c(O)c(=O)n1C)C(c1ccccc1)c1cc(F)ccc1Cl] | 88 From Isomer 2 eutomer | ESI-MS m/Z = 484.4 [M + H]+. Calculated MW: 483.9 CHIRAL- PAK AD- H(250*21) mm, 5 u; Isocratic: Line A:B (80:20); MeOH:ACN (50:50) in Liquid CO₂ + 0.1% DEA; Column Flow: 80 ml/min; RT of | ¹H NMR (400 MHz, DMSO- d₆): δ 2.61 (s, 3H), 3.65 (s, 3H), 6.19 (s, 1H), 7.05-7.62 (m, 8H), 8.92 (s, 1H), 9.30 (s, 1H), 10.17 (s, 1H), 11.00 (s, 1H). |
| | | Isomer-2: | |
| | | 4.98 min. | |
| 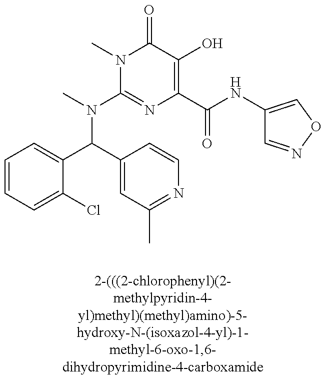 [SMILES: Cc1cc(C(c2ccccc2Cl)N(C)c2nc(C(=O)Nc3cnoc3)c(O)c(=O)n2C)ccn1] | 89 From Isomer 1 Distomer | ESI-MS m/Z = 481.3 [M + H]+. Calculated MW: 480.9 CHIRAL- CEL OX- H(250*21) mm, 5 u; Isocratic Line A:B (60:40); IPA:MEOH (50:50) in Hexanes + 0.1% DEA; Column Flow: 18 ml/min; | ¹H NMR (400 MHz, DMSO- d₆): δ 2.43 (s, 3H), 2.66 (s, 3H), 3.69 (s, 3H), 6.23 (s, 1H), 7.28 (t, J = 7.6 Hz, 1H), 7.37-7.48 (m, 4H), 7.73 (d, J = 7.6 Hz, 1H), 8.39 (d, J = 4.8 Hz, 1H), 8.96 (s, 1H), 9.34 (s, 1H), 10.40 (bs, 1H), 11.49 (bs, 1H). |
| | | RT of | |
| | | Isomer-1: | |
| | | 21.11 | |
| | | min. | |
| 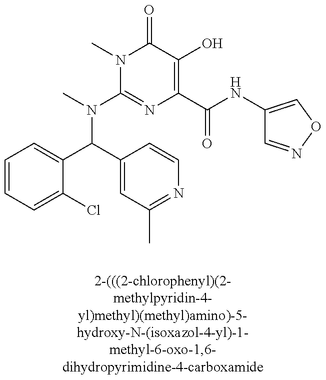 [SMILES: Cc1cc(C(c2ccccc2Cl)N(C)c2nc(C(=O)Nc3cnoc3)c(O)c(=O)n2C)ccn1] | 90 From Isomer 2 eutomer | ESI-MS m/Z = 481.3 [M + H]+. Calculated MW: 480.9 CHIRAL- CEL OX- H(250*21) mm, 5 u; Isocratic Line A:B (60:40); IPA:MEOH (50:50) in Hexanes + 0.1% DEA; Column Flow: 18 ml/min; | ¹H NMR (400 MHz, DMSO- d₆): δ 2.44 (s, 3H), 2.67 (s, 3H), 3.69 (s, 3H), 6.25, (s, 1H), 7.26 (t, J = 7.6 Hz, 1H), 7.36-7.49 (m, 4H), 7.73 (d, J = 7.6 Hz, 1H), 8.40 (d, J = 4.8 Hz, 1H), 8.96 (s, 1H), 9.34 (s, 1H), 10.22 (s, 1H), 11.09 (s, 1H). |
| | | RT of | |
| | | Isomer-2: | |
| | | 30.53 | |
| | | min. | |
| 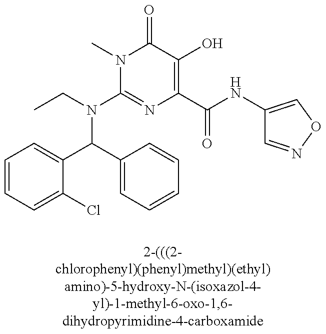 [SMILES: CCN(c1nc(C(=O)Nc2cnoc2)c(O)c(=O)n1C)C(c1ccccc1)c1ccccc1Cl] | 91 From Isomer 1 Distomer | ESI-MS m/Z = 480.3 [M + H]+. Calculated MW: 479.9 CHIRAL- PAK IB- N(250*21) mm, 5u; Isocratic Line A:B (85:15); MeOH in Liquid CO₂ + 0.1% DEA; Column Flow: 80 | ¹H NMR (400 MHz, DMSO- d₆): δ 0.96 (t, J = 6.8 Hz, 3H), 3.03 (q, J = 7.6, 1H), 3.16 (q, J = 7.2 Hz, 1H), 3.64 (s, 3H), 6.21 (s, 1H), 7.20 (d, J = 6.8 Hz, 2H), 7.28- 7.36 (m, 4H), 7.53 (d, J = 7.2 Hz, 2H), 7.77 (d, J = 7.6 Hz, 1H), 8.96 (s, 1H), 9.32 (s, 1H), 10.11 (s, 1H), 11.10 (s, 1H). |
| | | ml/min; | |
| | | RT of | |
| | | Isomer-1: | |
| | | 8.38 min. | |
| 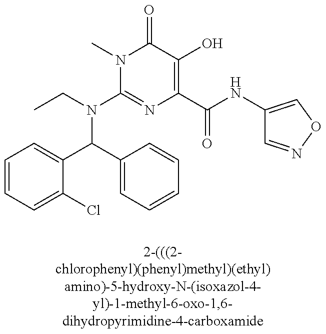 [SMILES: CCN(c1nc(C(=O)Nc2cnoc2)c(O)c(=O)n1C)C(c1ccccc1)c1ccccc1Cl] | 92 From Isomer 2 eutomer | ESI-MS m/Z = 480.3 [M + H]+. Calculated MW: 479.9 CHIRAL- PAK IB- N(250*21) mm, 5 u; Isocratic Line A:B (85:15); MeOH in Liquid CO₂ + 0.1% DEA; Column Flow: 80 | ¹H NMR (400 MHz, DMSO- d₆): δ 0.96 (t, J = 6.8 Hz, 3H), 3.02 (q, J = 7.2, 1H), 3.15 (q, J = 6.8 Hz, 1H), 3.63 (s, 3H), 6.21 (s, 1H), 7.20 (d, J = 7.2 Hz, 2H), 7.28- 7.36 (m, 4H), 7.52 (d, J = 7.6 Hz, 2H), 7.77 (d, J = 6.8 Hz, 1H), 8.95 (s, 1H), 9.31 (s, 1H), 10.14 (s, 1H), 11.11 (s, 1H). |
| | | ml/min; | |
| | | RT of | |
| | | Isomer-2: | |
| | | 10.77 min. | |
| 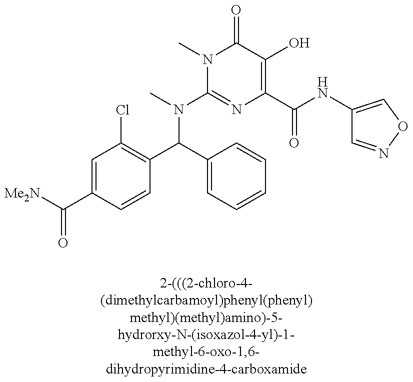 [SMILES: CN(C)C(=O)c1ccc(C(c2ccccc2)N(C)c2nc(C(=O)Nc3cnoc3)c(O)c(=O)n2C)c(Cl)c1] | 93 From Isomer 1 Distomer | ESI-MS m/Z = 537.4 [M + H]+. Calculated MW: 536.9 CHIRAL- PAK IC(250*21) mm, 5 u; Isocratic Line A:B (65:35); IPA:ACN (70:30) in Hexanes + 0.1% DEA; Column Flow: 20 ml/min; RT of | 1H NMR (400 MHz, DMSO- d₆): δ 2.62 (s, 3H), 2.78 (s, 3H), 2.91 (s, 3H), 3.64 (s, 3H), 6.21 (s, 1H), 7.22-7.26 (m, 1H), 7.32- 7.36 (m, 3H), 7.40 (s, 1H), 7.58 (d, J = 7.2 Hz, 2H), 7.73 (d, J = 8.0 Hz, 1H), 8.92 (s, 1H), 9.29 (s, 1H), 10.15 (bs, 1H), 11.01 (bs, 1H). |
| | | Isomer-1: | |
| | | 19.15 mi. | |
| 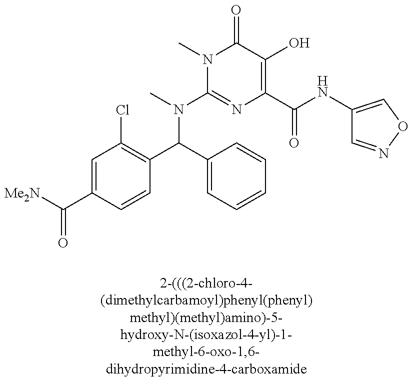 [SMILES: CN(C)C(=O)c1ccc(C(c2ccccc2)N(C)c2nc(C(=O)Nc3cnoc3)c(O)c(=O)n2C)c(Cl)c1] | 94 From Isomer 2 eutomer | ESI-MS m/Z - 537.4 [M + H]+. Calculated MW: 536.9 CHIRAL- PAK IC(250*21) mm, 5 u; Isocratic Line A:B (65:35); IPA:ACN (70:30) in Hexanes + 0.1% DEA; Column Flow: 20 ml/min; | ¹H NMR (400 MHz, DMSO- d₆): δ 2.62 (s, 3H), 2.78 (s, 3H), 2.91 (s, 3H), 3.63 (s, 3H), 6.21 (s, 1H), 7.22-7.26 (m, 1H), 7.32- 7.36 (m, 3H), 7.40 (s, 1H), 7.58 (d, J = 7.6 Hz, 2H), 7.73 (d, J = 8.0 Hz, 1H), 8.92 (s, 1H), 9.29 (s, 1H), 10.16 (bs, 1H), 11.01 (bs, 1H). |
| | | RT of | |
| | | Isomer-2: | |
| | | 25.65 min. | |
| 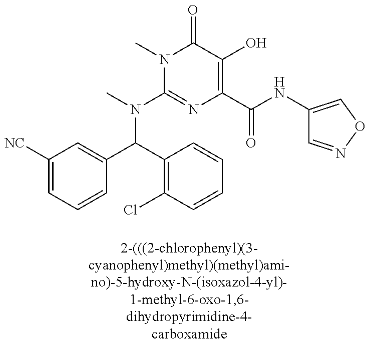 [SMILES: CN(c1nc(C(=O)Nc2cnoc2)c(O)c(=O)n1C)C(c1cccc(C#N)c1)c1ccccc1Cl] | 95 From Isomer 1 Distomer | ESI-MS m/Z = 491.2 [M + H]+. Calculated MW: 490.9 CHIRAL- CEL IC(250*21) mm, 5 u; Isocratic Line A:B (65:35); IPA:CAN (70:30) in Hexanes + 0.1% DEA; Column | ¹H NMR (400 MHz, DMSO- d₆): δ 2.59 (s, 3H), 3.65 (s, 3H), 6.26 (s, 1H), 7.25-7.26 (m, 1H), 7.35- 7.41 (m, 2H), 7.51 (t, J = 8.0 Hz, 1H), 7.68 (d, J = 7.2 Hz, 1H), 7.82 (d, J = 7.6 Hz, 1H), 7.86 (d, J = 7.6 Hz, 1H), 8.15 (s, 1H), 8.89 (s, 1H), 9.28 (s, 1H), 10.26 (s, 1H), 11.18 (bs, 1H). |
| | | Flow: 18 | |
| | | ml/min; | |
| | | RT of | |
| | | Isomer-1: | |
| | | 19.202 | |
| | | min. | |
| 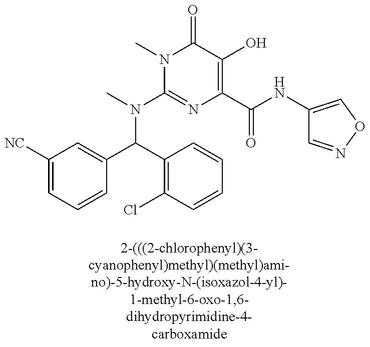 [SMILES: CN(c1nc(C(=O)Nc2cnoc2)c(O)c(=O)n1C)C(c1cccc(C#N)c1)c1ccccc1Cl] | 96 From Isomer 2 eutomer | ESI-MS m/Z = 491.3 [M + H]+. Calculated MW: 490.9 CHIRAL- CEL IC(250*21) mm, 5 u; Isocratic Line A:B (65:35); IPA:CAN (70:30) in Hexanes + 0.1% DEA; Column | ¹H NMR (400 MHz, DMSO- d6): δ 2.61 (s, 3H), 3.65 (s, 3H), 6.28 (s, 1H), 7.22-7.26 (m, 1H), 7.34- 7.40 (m, 2H), 7.52 (t, J = 8.0 Hz, 1H), 7.68 (d, J = 7.6 Hz, 1H), 7.80 (d, J = 7.6 Hz, 1H), 7.87 (d, J = 8.0 Hz, 1H), 8.08 (s, 1H), 8.91 (s, 1H), 9.29 (s, 1H), 10.26 (s, 1H), 11.05 (bs, 1H). |
| | | Flow: 18 | |
| | | ml/min; | |
| | | RT of | |
| | | Isomer-2: | |
| | | 28.546 | |
| | | min. | |
| 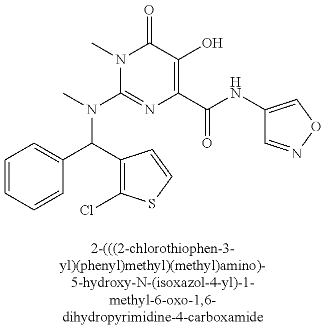 [SMILES: CN(c1nc(C(=O)Nc2cnoc2)c(O)c(=O)n1C)C(c1ccccc1)c1ccsc1Cl] | 97 From Isomer 1 Distomer | ESI-MS m/Z = 472.2 [M + H]+. Calculated MW: 471.1 | ¹H NMR (400 MHz, DMSO- d₆) δ 11.16 (s, 1H), 10.32 (s, 1H), 9.31 (s, 1H), 8.95 (s, 1H), 7.51-7.45 (m, 2H), 7.33 (t, J = 7.6 Hz, 2H), 7.23 (t, J = 7.4 Hz, 1H), 7.00 (d, J = 3.9 Hz, 1H), 6.93 (d, J = 3.8 Hz, 1H), 6.16 (s, 1H), 3.60 (s, 3H), 2.63 (s, 3H). |
| 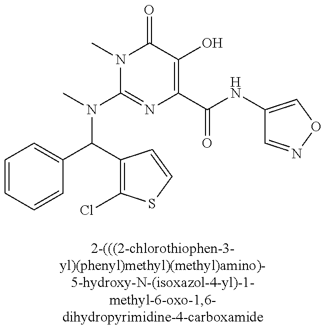 [SMILES: CN(c1nc(C(=O)Nc2cnoc2)c(O)c(=O)n1C)C(c1ccccc1)c1ccsc1Cl] | 98 From Isomer 2 eutomer | ESI-MS m/Z = 472.2 [M + H]+. Calculated MW: 471.1 | ¹H NMR (400 MHz, DMSO- d₆) δ 11.16 (s, 1H), 10.32 (s, 1H), 9.31 (s, 1H), 8.95 (s, 1H), 7.49 (d, J = 7.6 Hz, 2H), 7.33 (t, J = 7.5 Hz, 2H), 7.23 (t, J = 7.4 Hz, 1H), 7.00 (d, J = 3.8 Hz, 1H), 6.93 (d, J = 3.8 Hz, 1H), 6.15 (s, 1H), 3.60 (s, 3H), 2.63 (s, 3H). |

### Biochemical Assays

### 1. Silencing TREX1 in Tumor Cells

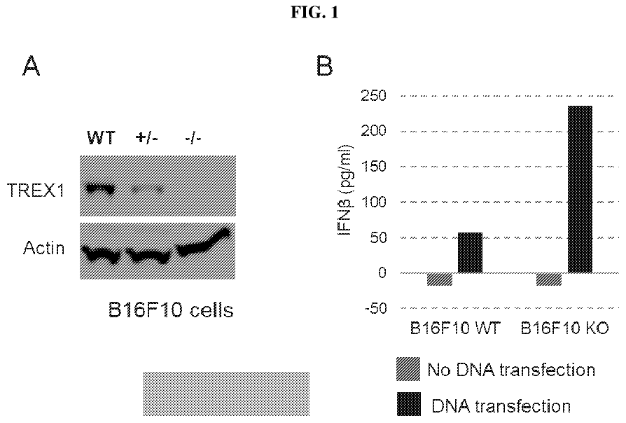

Activation of the cGAS/STING pathway upon sensing of cytosolic DNA and subsequent type I IFN production can occur in both tumor cells and innate immune cells, particularly dendritic cells. To evaluate whether TREX1 keeps in check the production of type I IFN by a well described, cold syngeneic tumor model that undergoes immune-mediated rejection upon activation of type I IFN by STING agonists, TREX1 was knocked down in B16F10 tumor cells using CRISPR (FIG. 1A). Accumulation of cytosolic DNA via DNA transfection of the tumor cells resulted in an about 5-fold increase in IFNβ production by the TREX1 knockout B16F10 cells relative to the parental tumor cells, demonstrating that TREX1 attenuated the activation of the cGAS/STING pathway in B16F10 tumor cells (FIG. 1B).

### 2. Growth of TREX1-Competent and -Deficient B16F10 Tumor Cells In Vivo

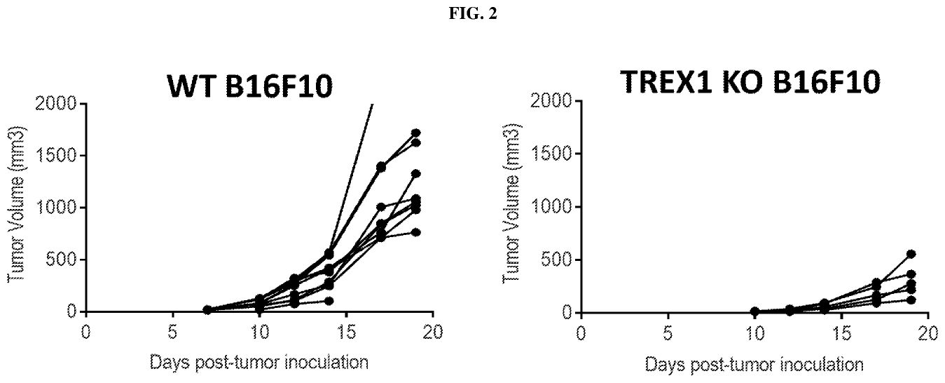

The growth of TREX1-competent and -deficient B16F10 tumor cells in vivo was evaluated. C57BL/6J mice were inoculated subcutaneously on the right flank with 300,000 parental or TREX1 knockout B16F10 tumor cells. Body weights were collected two times per week, and tumor measurements, two to three times per week, starting when tumors became measurable and for the remaining duration of the study. Tumors in which TREX1 had been silenced presented with remarkably smaller volumes than the parental B16F10 tumors (FIG. 2).

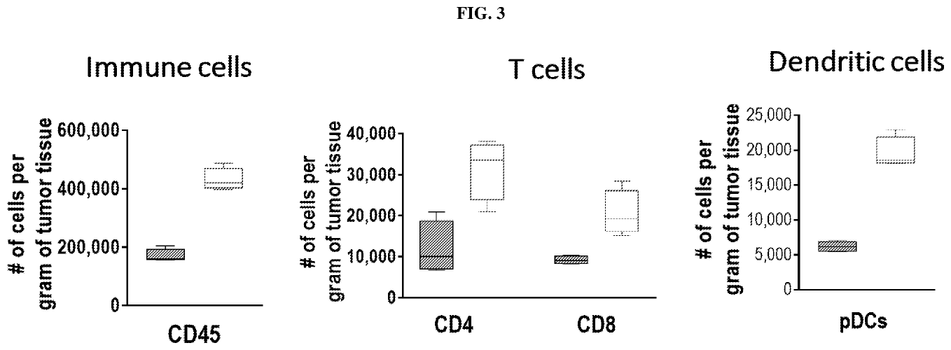

Tumors were harvested on day 19, upon termination of the study, and digested into single cell suspensions to enable flow cytometry quantification of tumor-infiltrating immune populations. TREX1 knockout B16F10 tumors were found to exhibit a significant increase in overall immune cells, which reflected an increase in the number of tumor infiltrating CD4 and CD8 T cells as well as in plasmacytoid dendritic cells (pDCs) (FIG. 3). pDCs are known to play a central role in the induction of antigen-specific anti-tumor immune responses whereas T cells are known to be major effectors of anti-tumor efficacy in mice and humans. The profound change in the immune infiltrate of the tumors deficient in TREX1 thus suggest that the inhibition of the growth of the latter tumors is at least in part immune-mediated.

### TREX1 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged full length human TREX1 (expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10 point dose-response in DMSO. To this was added 7.5 μL of dsDNA substrate (Strand A: 5′ TEX615/GCT AGG CAG 3′; Strand B: 5′ CTG CCT AGC/IAbRQSp (Integrated DNA Technologies)) in reaction buffer. Final concentrations were 150 pM TREX1, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction were 112.5 pM TREX1, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

### TREX2 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged human TREX2 (residues M44-A279, expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10 point dose-response in DMSO. To this was added 7.5 μL of dsDNA substrate (Strand A: 5′ TEX615/GCT AGG CAG 3′; Strand B: 5′ CTG CCT AGC/IAbRQSp (IDT)) in reaction buffer. Final concentrations were 2.5 nM TREX2, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction mixture were 1.875 pM TREX2, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

Results are shown in Table 1. TREX1 IC₅₀: A=<0.1 μM; B=0.1 to 1 μM; C=1 to 10 μM; D=>10 μM. TREX2 IC₅₀: A=<1 μM, B=1 to 10 μM, C=10 to 100 μM, D=>100 μM.

TABLE 1

| Example | TREX1 IC₅₀ | TREX2 IC₅₀ |
|---|---|---|
| 1 | A | A |
| 2 | A | B |
| 3 | B | C |
| 4 | A | A |
| 5 | A | B |
| 6 | A | B |
| 7 | A | B |
| 8 | A | B |
| 9 | A | C |
| 10 | A | B |
| 11 | A | B |
| 12 | B | C |
| 13 | A | B |
| 14 | A | B |
| 15 | A | B |
| 16 | A | A |
| 17 | A | B |
| 18 | A | B |
| 19 | A | B |
| 20 | B | B |
| 22 | B | B |
| 25 | A | B |
| 27 | A | A |
| 28 | A | B |
| 29 | A | B |
| 30 | A | A |
| 31 | A | B |
| 32 | A | B |
| 33 | B | C |
| 34 | B | C |
| 35 | A | B |
| 37 | B | C |
| 38 | B | B |
| 39 | A | A |
| 40 | A | A |
| 41 | A | A |
| 42 | A | B |
| 43 | A | A |
| 44 | A | A |
| 45 | A | B |
| 46 | A | B |
| 50 | A | A |
| 49 | B | C |
| 51 | A | A |
| 52 | A | B |
| 53 | A | A |
| 54 | A | A |
| 56 | A | A |
| 57 | A | A |
| 58 | A | B |
| 59 | A | A |
| 60 | A | B |
| 61 | A | A |
| 62 | A | A |
| 63 | A | A |
| 66 | A | A |
| 67 | A | A |
| 68 | A | A |
| 69 | A | A |
| 70 | A | A |
| 71 | A | A |
| 72 | B | B |
| 73 | B | C |
| 74 | A | B |
| 75 | B | C |
| 76 | B | C |
| 77 | B | C |
| 78 | A | B |
| 79 | A | B |
| 80 | A | B |
| 81 | A | A |
| 82 | D | D |
| 83 | A | A |
| 84 | A | A |
| 85 | A | A |
| 86 | A | A |
| 87 | A | A |
| 88 | A | A |
| 89 | A | A |
| 90 | A | A |
| 91 | A | A |
| 92 | A | A |
| 93 | A | B |
| 94 | A | A |
| 95 | A | A |
| 96 | A | A |

### HCT116 Cell Assay

HCT116 dual cells (Invivogen, San Diego, Calif., USA) are derived from the human HCT116 colorectal carcinoma cell line. Cells have been selected for the stable integration of SEAP and Luciferase reporter genes, which expression is under the control of 5 tandem response elements for NF-KB/AP1 and STAT1/STAT2, respectively. The cell line was used to monitor Type I interferon induction and subsequent signaling by measuring the activity of the Lucia luciferase secreted in the culture medium.

HCT116 cells were plated in 96-well plate(s) at 40,000 cells/well in 100 uL DMEM supplemented with 10% FBS and 25 mM Hepes (pH 7.2-7.5). After overnight settling, cells were treated with TREX1i for 4 h (maximum DMSO fraction was 0.1%) before 1.25 ug/mL pBR322/BstNI restriction digest (New England Biolabs, Ipswich, Mass., USA) was transfected with Lipofectamine LTX (ThermoFisher, Grand Island, N.Y., USA), according to product manual recommendations. Briefly, Lipofectamine LTX (0.4 uL/well) was diluted in OptiMEM (5 uL/well). pBR322/BstNI (100 ng/well) was diluted in OptiMEM (5 uL/well) before Plus reagent (0.1 uL/100 ng DNA) was added. After 5 min incubation at room temperature, the DNA mixture was mixed dropwise with the diluted Lipofectamine LTX. After an additional 10 min incubation, the transfection mix (10 uL/well) was added to the cells. Cells were maintained at 37° C. for 48 h before monitoring the Lucia Luciferase activity from the cell culture medium. EC₅₀ values were calculated by comparing the measured luminescence relative to 10 uM compound 39 (100% inhibition) and no inhibitor (0% inhibition) controls using non-linear least-squares four parameter fits in either Genedata Screener or GraphPad Prism (GraphPad Software, Inc.).

Results are shown in Table 1. TREX1 IC₅₀: A=<1.0 μM; B=1.0 to 10 μM; C=10 to 100 μM

TABLE 2

| Example | HCT116 EC₅₀ |
|---|---|
| 1 | B |
| 2 | B |
| 3 | — |
| 4 | B |
| 5 | B |
| 6 | B |
| 7 | B |
| 8 | B |
| 9 | C |
| 10 | C |
| 11 | B |
| 12 | — |
| 13 | C |
| 14 | C |
| 15 | B |
| 16 | C |
| 17 | — |
| 18 | C |
| 19 | C |
| 20 | — |
| 22 | C |
| 25 | C |
| 27 | B |
| 28 | B |
| 29 | C |
| 30 | A |
| 31 | B |
| 32 | C |
| 33 | — |
| 34 | — |
| 35 | C |
| 37 | — |
| 38 | — |
| 39 | A |
| 40 | A |
| 41 | A |
| 42 | B |
| 43 | B |
| 44 | A |
| 45 | B |
| 46 | B |
| 50 | A |
| 49 | — |
| 51 | B |
| 52 | C |
| 53 | A |
| 54 | A |
| 56 | A |
| 57 | A |
| 58 | B |
| 59 | A |
| 60 | B |
| 61 | A |
| 62 | A |
| 63 | B |
| 66 | A |
| 67 | A |
| 68 | A |
| 69 | A |
| 70 | B |
| 71 | A |
| 72 | — |
| 73 | — |
| 74 | B |
| 75 | — |
| 76 | — |
| 77 | — |
| 78 | C |
| 79 | C |
| 80 | C |
| 81 | A |
| 82 | — |
| 83 | A |
| 84 | A |
| 85 | A |
| 86 | A |
| 87 | A |
| 88 | A |
| 89 | A |
| 90 | B |
| 91 | B |
| 92 | B |
| 93 | B |
| 94 | A |
| 95 | A |
| 96 | B |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 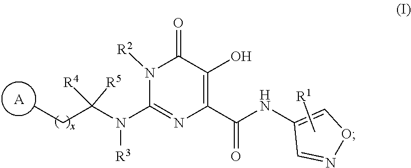
or a pharmaceutically acceptable salt thereof, wherein:
R¹ is hydrogen, (C₁-C₄)alkyl, halo(C₁-C₄)alkyl, 3- to 4-membered cycloalkyl, —OR^{f}, —SR^{f}, or —NR^{e}R^{f};
R² is hydrogen, (C₁-C₄)alkyl, halo(C₁-C₄)alkyl, or 3- to 4-membered cycloalkyl;
R³ is hydrogen or (C₁-C₄)alkyl optionally substituted with phenyl, wherein said phenyl is optionally substituted with 1 to 3 groups selected from halo, (C₁-C₄)alkyl, and halo(C₁-C₄)alkyl;
R⁴ is hydrogen or (C₁-C₄)alkyl;
R⁵ is hydrogen, aryl, heteroaryl, heterocyclyl, cycloalkyl, phenyl, or (C₁-C₄)alkyl optionally substituted with phenyl or —NHC(O)OR^{a}, wherein each of said phenyl is optionally and independently substituted with 1 to 3 groups selected from halo, (C₁-C₄)alkyl, and halo(C₁-C₄)alkyl;
x is 0, 1, or 2;
Ring A is aryl, heteroaryl, heterocyclyl, or cycloalkyl, each of which are optionally and independently substituted with 1 or 2 groups selected from R⁶;
R⁶ is (C₁-C₄)alkyl, halo(C₁-C₄)alkyl, halo(C₁-C₄)alkoxy, halo, phenyl, —CN, —NHC(O)OR^{a}, —NHC(S)OR^{a}, —C(O)R^{b}, —NHC(O)NHR^{g}, —NHC(S)NHR^{g}, —NHS(O)₂NHR^{g}, —C(S)R^{b}, —S(O)₂R^{c}, —S(O)R^{c}, —C(O)OR^{d}, —C(S)OR^{d}, —C(O)NR^{e}R^{f}, —C(S)NHR^{e}, —NHC(O)R^{d}, —NHC(S)R^{d}, —OR′, —SR^{e}, —O(C₁-C₄)alkylOR^{e}, —NR^{e}R^{f}, 4- to 6-membered heteroaryl, or 4- to 7-membered heterocyclyl, wherein
said phenyl for R⁶ is optionally substituted with 1 or 2 groups selected from R^{g};
said (C₁-C₄)alkyl for R⁶ is optionally substituted with 1 or 2 groups selected from OR^{h}, —NR^{j}R^{k}, phenyl, and 5- to 6-membered heteroaryl; and
said 4- to 7-membered heterocyclyl and 4- to 6-membered heteroaryl for R⁶ are each optionally and independently substituted with 1 or 2 groups selected from R^{m}; and
wherein said phenyl and 5- to 6-membered heteroaryl of the optional substituents listed for (C₁-C₄)alkyl in R⁶ are each optionally and independently substituted with 1 or 2 groups selected from R^{g};
R^{g}, R^{h}, R^{j}, R^{k}, and R^{m} are each independently hydrogen, halo, (C₁-C₄)alkyl, halo(C₁-C₄)alkyl, (C₁-C₄)alkoxy, halo(C₁-C₄)alkoxy, phenyl, —(C₁-C₄)alkylphenyl, 3- to 4-membered cycloalkyl, 4- to 6-membered heteroaryl, or 4- to 7-membered heterocyclyl, and wherein said 4- to 7-membered heterocyclyl for R^{g}, R^{h}, R³ and R^{k} is further optionally substituted with ═O.
R^{a}, R^{b}, R^{c}, R^{d}, R^{e} and R^{f} are each independently hydrogen, halo, (C₁-C₄)alkyl, halo(C₁-C₄)alkyl, (C₁-C₄)alkoxy, halo(C₁-C₄)alkoxy, phenyl, 3- to 4-membered cycloalkyl, 4-to 6-membered heteroaryl, or 4- to 7-membered heterocyclyl, wherein
said (C₁-C₄)alkyl for R^{a}, R^{b}, R^{c}, R^{d}, R^{e} and R^{f} is optionally substituted with 1 or 2 groups selected from phenyl, —OR^{h}, —NR^{j}R^{k};
said phenyl, 4- to 6-membered heteroaryl, and 4- to 7-membered heterocyclyl for R^{a}, R^{b}, R^{c}, R^{d}, R^{e}, and R^{f} are each optionally and independently substituted with 1 or 2 groups selected from R^{g}; and
said 4- to 7-membered heterocyclyl for R^{a}, R^{b}, R^{e}, R^{d}, R^{e}, and R^{f} is further optionally substituted with ═O.

2. The compound of claim 1, wherein the compound is of the Formula II: 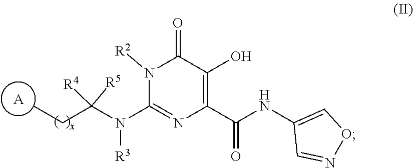
or a pharmaceutically acceptable salt thereof.

3. The compound of claim 1 or 2, wherein R² is (C₁-C₄)alkyl.

4. The compound of any one of claims 1 to 3, wherein the compound is of the Formula III: 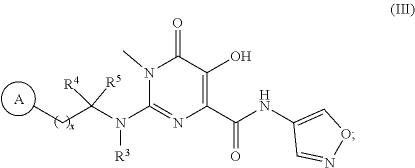
or a pharmaceutically acceptable salt thereof.

5. The compound of any one of claims 1 to 4, wherein R³ is (C₁-C₄)alkyl optionally substituted with phenyl.

6. The compound of any one of claims 1 to 5, wherein R³ is (C₁-C₄)alkyl.

7. The compound of any one of claims 1 to 6, wherein the compound is of the Formula IV: 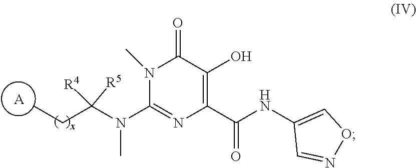
or a pharmaceutically acceptable salt thereof.

8. The compound of any one of claims 1 to 7, wherein the compound is of the Formula V: 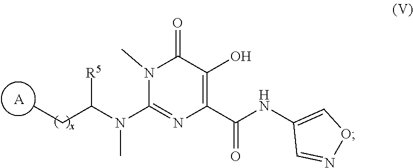
or a pharmaceutically acceptable salt thereof.

9. The compound of any one of claims 1 to 8, wherein x is 0 or 1.

10. The compound of any one of claims 1 to 9, wherein R⁵ is hydrogen, aryl, heteroaryl, heterocyclyl, cycloalkyl, phenyl, or (C₁-C₄)alkyl optionally substituted with phenyl or —NHC(O)OR^{a}.

11. The compound of any one of claims 1 to 10, wherein R⁵ is hydrogen, phenyl, or (C₁-C₄)alkyl optionally substituted with phenyl or —NHC(O)OR^{a}.

12. The compound of any one of claims 1 to 11, wherein R^{a} is (C₁-C₄)alkyl.

13. The compound of any one of claims 1 to 10, wherein R⁵ is cycloalkyl or phenyl, wherein said phenyl is optionally substituted with 1 to 3 groups selected from halo, (C₁-C₄)alkyl, and halo(C₁-C₄)alkyl.

14. The compound of any one of claims 1 to 10, wherein R⁵ is cyclopropyl.

15. The compound of any one of claims 1 to 10, wherein R⁵ is phenyl optionally substituted with 1 to 2 groups selected from halo and (C₁-C₄)alkyl, and halo(C₁-C₄)alkyl.

16. The compound of any one of claims 1 to 10, wherein R⁵ is phenyl optionally substituted with 1 to 2 halo.

17. The compound of any one of claims 1 to 16, wherein ring A is aryl, heteroaryl, or heterocyclyl, each of which are optionally and independently substituted with 1 or 2 groups selected from R⁶.

18. The compound of any one of claims 1 to 17, wherein ring A is naphthalenyl, indazolyl, phenyl, pyridyl, pyrazolyl, azetidinyl, tetrahydropyranyl, piperidinyl, dihydrobenzooxazinyl, dihydrobenzodioxinyl, or chromanyl, each of which are optionally and independently substituted with 1 or 2 groups selected from R⁶.

19. The compound of any one of claims 1 to 18, wherein ring A is phenyl optionally substituted with 1 or 2 groups selected from R⁶.

20. The compound of any one of claims 1 to 17, wherein ring A is pyrimidinyl or thiazolyl each optionally substituted with 1 or 2 groups selected from R⁶.

21. The compound of any one of claims 1 to 17, wherein ring A is pyridyl optionally substituted with 1 or 2 groups selected from R⁶.

22. The compound of any one of claims 1 to 21, wherein R⁶ is halo(C₁-C₄)alkyl, halo, —CN, —NHC(O)OR^{a}, —C(O)R^{b}, —NHC(O)NHR^{g}, —C(O)NR^{e}R^{f}, —NHC(O)R^{d}, —NR^{e}R^{f}, —OR^{e}, or 4-to 6-membered heteroaryl, wherein said 4- to 6-membered heteroaryl is optionally substituted with 1 or 2 groups selected from R^{m}.

23. The compound of any one of claims 1 to 22, wherein R⁶ is (C₁-C₄)alkyl, halo(C₁-C₄)alkyl, halo, —CN, —C(O)R^{b}, —C(O)NR^{e}R^{f}, —OR^{e}, or 4- to 6-membered heteroaryl, wherein said 4- to 6-membered heteroaryl is optionally substituted with 1 or 2 groups selected from R^{m}.

24. The compound of any one of claims 1 to 23, wherein R⁶ is phenyl or 4- to 6-membered heteroaryl, wherein said phenyl for is optionally substituted with 1 or 2 groups selected from R^{g} and said 4- to 6-membered heteroaryl is optionally substituted with 1 or 2 groups selected from R^{m}.

25. The compound of any one of claims 1 to 24, wherein R^{b} is (C₁-C₄)alkyl.

26. The compound of any one of claims 1 to 25, wherein R^{e} is (C₁-C₄)alkyl.

27. The compound of any one of claims 1 to 26, wherein R^{f} is (C₁-C₄)alkyl.

28. The compound of any one of claims 1 to 27, wherein R^{m} is (C₁-C₄)alkyl.

29. The compound of any one of claims 1 to 28, wherein R^{g} is halo.

30. The compound of any one of claims 1 to 28, wherein R⁶ is Cl, F, CF₃, —C(O)N(Me)₂, —OCH₃, —C(O)CH₃, or pyrazolyl optionally substituted with 1 or 2 CH₃.

31. A pharmaceutical composition comprising the compound of any one of claims 1 to 30, or a pharmaceutically acceptable salt thereof; and a pharmaceutically acceptable carrier.

32. A method of treating a disease responsive to the inhibition of TREX1 in a subject, comprising administering to the subject, a therapeutically effective amount of a compound of any one of claims 1 to 30, or a pharmaceutically acceptable salt thereof, or the composition of claim 31.

33. The method of claim 32, wherein the disease is cancer.
